(19) United States
(12) Patent Application Publication
(10) Pub. No.: US 2022/0072138 A1
(43) Pub. Date: Mar. 10, 2022

(54) NRTI THERAPIES

(71) Applicants: THE UNIVERSITY OF LIVERPOOL, Liverpool (GB); THE JOHNS HOPKINS UNIVERSITY, Baltimore, MD (US)

(72) Inventors: Andrew OWEN, Liverpool (GB); Steve RANNARD, Liverpool (GB); Faye HERN, Liverpool (GB); Chung LIU, Liverpool (GB); Anika SHAKIL, Liverpool (GB); Megan NEARY, Liverpool (GB); Caren Freel MEYERS, Baltimore, MD (US); Kartik TEMBURNIKAR, Baltimore, MD (US); Lauren BAMBARGER, Baltimore, MD (US); Stephanie HENRIQUEZ, Baltimore, MD (US)

(21) Appl. No.: 17/416,350

(22) PCT Filed: Dec. 20, 2019
(86) PCT No.: PCT/GB2019/053678

§ 371 Date: Jun. 18, 2021

Related U.S. Application Data
(60) Provisional application No. 62/783,337, filed on Dec. 21, 2018.

(51) Int. Cl. A61K 47/55; A61K 47/60; A61K 47/69

(52) U.S. Cl. CPC A61K 47/55; A61K 47/6921; A61K 47/60

(57) ABSTRACT

Polymer-of-prodrug (POP) materials enable new nucleoside reverse transcriptase inhibitor (NRTI) therapy strategies. The materials are prodrugs of NRTIs in the form of polymers. Suitable materials include products which are polymeric NRTI delivery systems comprising polymeric materials which are capable of degradation after administration to release NRTIs or NRTI prodrugs which themselves are capable of metabolism to the parent NRTIs. The NRTIs may optionally be selected from tenofovir (TFV), emtricitabine (FTC), lamivudine (3TC) and MK-8591 (EFdA). The invention facilitates long-acting (LA) regimens. Constructs of the materials may be in the form of injectable compositions or implants.

The present invention relates to prodrugs of nucleoside reverse transcriptase inhibitors (NRTIs) and polymeric delivery systems for the same.

Antiretroviral therapy (ART) involves long-term co-administration of several drug classes to simultaneously engage multiple HIV viral targets, maximizing inhibition of viral replication and minimizing drug resistance emergence. UNAIDS statistics reported that ˜37 million people were estimated to be living with HIV infection globally in 2015 (including 1.8 million children) and 1.1 million AIDS-related deaths occurred in 2015 alone. Since the start of the epidemic, ˜35 million people have died and 78 million people have been infected. Access to ART has risen to ˜17 million patients over recent years¹ and, to date, 6 classes of antiretroviral drugs (ARVs) are available: nucleoside/nucleotide reverse transcriptase inhibitors (NRTIs); non-nucleoside reverse transcriptase inhibitors (NNRTI); protease inhibitors (PIs); fusion inhibitors; CCR5 antagonists and integrase inhibitors. Although ART regimens have been highly successful in moderating morbidity and mortality rates, therapeutic failure is reported in ˜8% of treatment naïve and 33% of treatment experienced patients.² ARV use for post- and pre-exposure prophylaxis (PrEP) alms to control transmission rates.

A complex range of issues jeopardize ART efficacy, contributing to the established heterogeneity of clinical response to ARVs. These include viral characteristics, immunological status, pharmacokinetic (PK) variability to drug exposure and sub-optimal patient adherence to regimens. Currently, therapy necessitates lifelong, daily dosing and successful adherence may be determined by the interplay of multiple factors³ ranging from lifestyle and underlying co-morbidities to employment status, age or gender. Poor adherence places patients at risk of treatment failure and low rates of protection for PrEP.⁴

The potential impact of emerging technologies⁵ to improve adherence with long-term therapy has been reviewed across a range of health conditions⁶ and includes approaches such as text alerts, peer-to-peer communication, cloud-based support and self-reporting smart pills. Unfortunately, many of these strategies are not relevant in the geographies bearing the major burden of the HIV; ˜25.5 million people are living with HIV in sub-Saharan Africa. The use of long-acting (LA) nanoparticle-derived intramuscular depot injections has shown extended/controlled drug release of 1-3 months after a single administration, maintaining therapeutic plasma drug concentrations, for an NNRTI (rilpivirine) and an integrase inhibitor (cabotegravir) in human trials.⁷⁻⁹ The combination of rilpivirine and cabotegravir injections shows promise in maintaining viral suppression in the LATTE 2 study,¹⁰ and this year entered phase III trials. However, patients might still be required to ingest a daily NRTI backbone therapy if LA NRTI drugs cannot be developed, which would undermine benefits for adherence. Non-adherence to any oral therapy component combined with either single LA regimen would result in monotherapy from the sustained release agent, increasing the likelihood for emergence of resistance. Complete sustained release regimens are urgently required and/or robust NRTI-sparing regimens must be thoroughly investigated (rilpivirine and cabotegravir). LA drug delivery exists in other therapeutic areas through the use of depot injections (contraception, mental health), drug-eluting implants (contraception, osteomyelitis) and drug-filled polymer nanoparticles (cancer). However, LA delivery of NRTIs is lacking.

Interest in developing polymer prodrugs for drug delivery is heightened, especially after the clinical success of pegylated drugs¹¹ with enhanced circulation half-lives (e.g. Adagen, Oncaspar, PEG-Intron & Pegasys). Indeed, in 2013 two of the top 10 selling pharmaceuticals were polymer prodrugs (Neulasta® and Copaxone®).¹² Polymer prodrugs generally address poor drug solubility and are injected intravenously, targeting specific disease sites.^{13,14} Biodegradable polymers incorporating drug molecules as monomer repeat units are reported with polyAspirin¹⁵ and polyMorphine.¹⁶ Despite progress in polymer therapeutics, very few reports describe polymer prodrugs for LA formulations of water-soluble drugs or to address the specific clinical needs of HIV therapy adherence or prophylaxis. Anionic polymeric sequestrants acting as topical/vaginal entry inhibitors (e.g. Starpharma's dendritic VivaGel) have been evaluated as PrEP candidates but failed to provide protection in human trials.¹⁷ Pegylated proteins,¹⁸ pegylated drugs (maraviroc)¹⁹ and polymer-zidovudine²⁰ conjugates are recently reported with the aim of targeting infection pathways within the systemic circulation, but little is discussed regarding administration routes or frequency of dosing intervals, and no actual drug release is targeted from these systems.

The present invention differs from disclosures in other documents including documents which: a) outline incorporation of pure TAF powder into an implantable silicone tube;⁷⁴ b) outline formation of PLGA-NRTI polymer conjugates for vaginal gels;⁷⁵ and c) review sustained release tablets, ceramic implants, solid drug nanoparticles, nanocontainers, liposomes, emulsomes, aspasomes, microemulsions and nanopowders.⁷⁶

From a first aspect the present invention provides a product which is a prodrug of a nucleoside reverse transcriptase inhibitor in the form of a polymer.

The product is a polymeric NRTI delivery system comprising a polymeric material which is capable of degradation after administration to release an NRTI or NRTI prodrug which itself is capable of metabolism to the parent NRTI.

We refer to these polymeric materials as “Polymer-of-Prodrug” (POP) materials.

The materials may be considered biodegradable polymeric NRTI delivery systems.

The invention is particularly useful when the NRTIs are water-soluble NRTIs.

The NRTIs may optionally be selected from tenofovir (TFV), emtricitabine (FTC), lamivudine (3TC) and MK-8591 (EFdA)).

The “Polymer-of-Prodrug” (POP) materials may undergo complete, sustained biodegradation after administration, releasing NRTIs or NRTI prodrugs capable of metabolism to parent NRTIs.

Thus, the NRTIs may be directly released from the polymer or alternatively the polymer may break down into fragments (which may themselves be considered prodrugs) which then release the NRTIs.

The present invention facilitates LA regimens, including LA regimens which are supported by existing efficacy data of the parent drugs in combination with cabotegravir²¹ or rilpivirine.²²

Experimental work presented below illustrates how POP structures can be prepared incorporating various NRTIs (e.g. FTC, 3TC, TAF and EFdA), incorporating various polymeric linker moieties, and using various synthetic routes.

The present invention utilises a prodrug approach. In the case of NRTIs such as emtricitabine (FTC), prodrugs may comprise functionalisation at the amine and hydroxy moieties, for example in the form of carbamates and carbonates. Cleavage at these positions releases the parent drug:

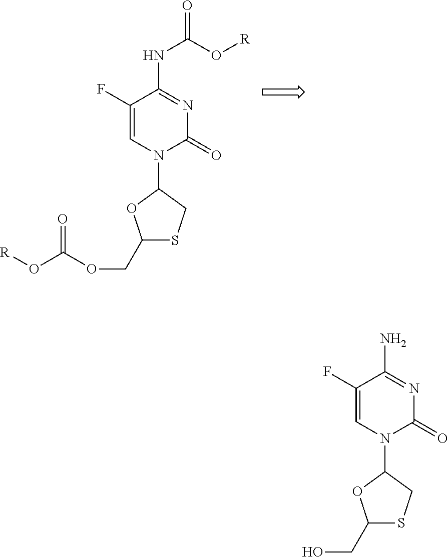

The products of the present invention differ from conventional prodrugs in that the drug is chemically bound within a polymeric structure (in the scheme below, B vs A):

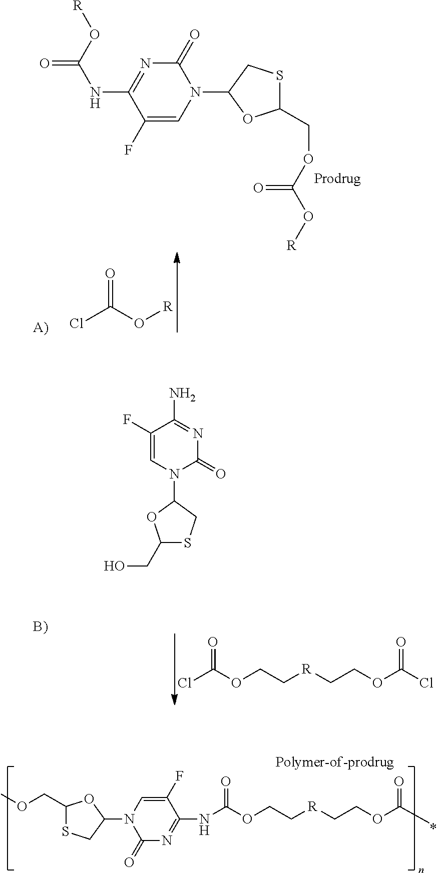

Several architectures are possible including for example those containing pendant prodrug moieties (e.g. by virtue of using multifunctional linkers):

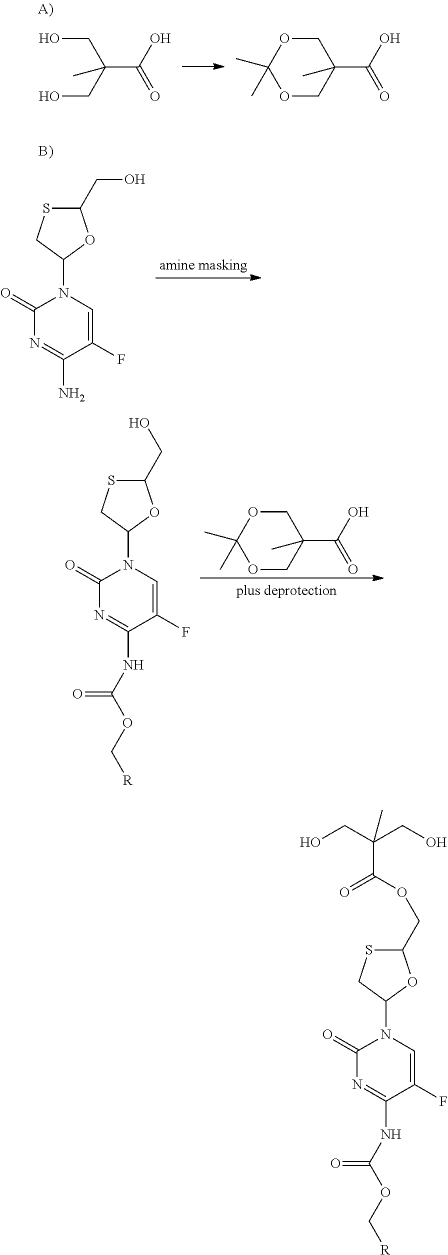

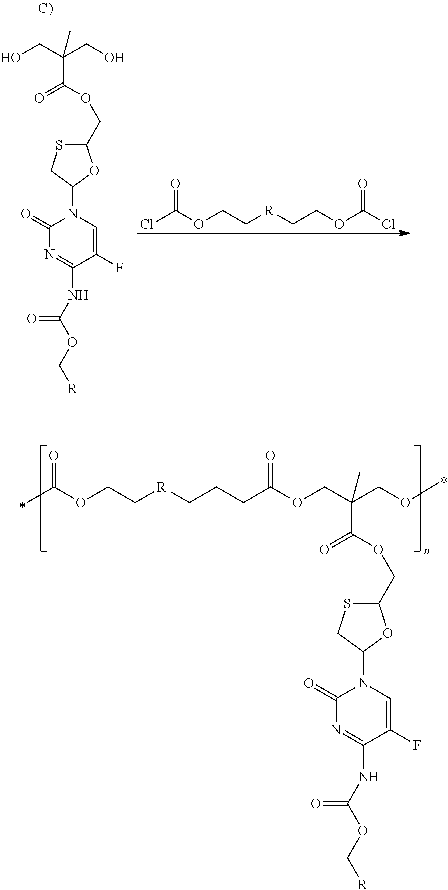

A) Synthesis of masked diol linker
B) Synthesis of dial functional carbamate prodrug
C) Polycarbonate POP synthesis

The POP structures of the present invention are polymers; these break down to form POP fragments during degradation.

POP constructs are products such as implants or nanoparticles containing the POP structures.

Where “R”, is used herein to denote moieties within chemical structures the skilled person will understand that each occurrence of “R” can denote the same or different groups.

In particular moieties which link NRTI residues (for example between ester, carbonate, amide or carbamate groups) may comprise various structures. These may include aliphatic or aromatic or heteroaromatic structures including chains such as chains which may comprise two or more carbon atoms, for example C₂-C₁₂ alkyl chains or chains which may comprise one or more aromatic or heteroaromatic ring. The skilled person will understand that various chemistries, functionalities and substituents may be present which are compatible with the underlying principle of incorporation of NRTI structures into polymeric structures.

Prior to the present invention the development of injectable LA options for HIV therapy had created a new paradigm that could significantly impact the dosing frequency for patients, to overcome adherence issues and generate considerable lifestyle benefits. The LATTE 2 trial¹⁰ had shown that separate, concurrent injections of rilpivirine and cabotegravir suppress viral replication. However, separately these LA options require an oral backbone therapy to provide robust protection.

The present invention provides NRTI-derived LA products that for example extend the scope for rilpivirine and cabotegravir LA. Since rilpivirine and cabotegravir are in different classes, they exhibit different resistance profiles; thus separate regimens provide first-line and second-line LA options.

Rilpivirine and cabotegravir LA are nanomedicines, comprising particles of water-insoluble drugs (solid drug nanoparticles, SDNs) suspended in a vehicle for injection. The SDNs are formed by nanomilling of larger dispersions; attrition in the milling process reduces the dispersion particle diameter to <1 micron. NRTIs are water-soluble and thus are not amenable to aqueous milling procedures as the presence of stabilizers, energy, increased surface area and the aqueous milling environment causes dissolution of the SDNs as they form. Although they are water-soluble, NRTIs do not exhibit high enough solubility to form solutions in injectable vehicles with concentrations that would minimize injections to clinically relevant volumes.

The present invention provides a new strategy to establish new routes to NRTI LA candidates, using polymers containing bioactive monomers.

The formation of polymers from drug molecules bearing multiple functional groups (Polymer-of-Drug (POD) approach) is reported to extend drug release for >2 weeks in rodent models.¹⁵ These systems rely on polyester-anhydride chemistry generated under conventional step-growth polymerization conditions, and release occurs over relatively short timescales. The formation of plaques for implantation allows high drug content and localized delivery, often toward a goal to improve bone repair. Intraperitoneal injection was also demonstrated after suspension of the polymeric drugs in an aqueous solution of stabilizers.¹⁶ A major benefit of polymers generated from drug molecules is polymer biodegradation to biologically relevant subunits and low molecular weight linker molecules. In contrast, drug eluting implants, in which the implant scaffold is not degraded during drug release, must be removed or replaced, often through a minor surgical procedure.²³

To date, the POD approach has been largely restricted to non-steroidal antiinflammatory drugs and there are no reports of ARV-based polymers. Polymers bearing pendant drug molecules are reported and are often prepared by free-radical polymerization approaches.²⁴ Pendant drug molecules are cleaved from the poorly-degradable backbone after I.V. injection, and prolonged systemic circulation and/or accumulation at target sites. Pegylated drugs are an exception to this general strategy as poly(ethylene glycol) chains (PEG) are grafted to drug molecules and latterly cleaved.²⁵ In both cases, the polymeric structure is maintained during and after drug release.

Prodrug approaches can overcome several issues including parent drug solubility, suboptimal PK profiles and poor cellular and tissue absorption. The formation of polymeric prodrugs (pendant or backbone) enables the generation of solid monolithic structures that may act as implants, or processing to form reproducible nanoparticle structures. Further chemical variables, in addition to prodrug variation, are also introduced to manipulate and optimize drug release through polymer degradation to monomeric parent drug structures and linkers.

Products of the present invention can be prepared by step-growth polymerization methods. Step-growth polymerization²⁶⁻²⁸ incorporates multifunctional monomers in chains of repeating structures. In their simplest form, bifunctional monomers may contain two complementary reactive groups in an A-B arrangement (e.g. a hydroxyl-acid for polyester synthesis); or, two monomers may individually bear the complementary functional groups, a so-called A₂/B₂ monomer combination (e.g. a diol (A₂) and a diacid (B₂) for polyester synthesis). Where orthogonal chemistry is present in either A-B or A₂/B₂ monomers, complementary linker chemistry is used for polymer chain formation.

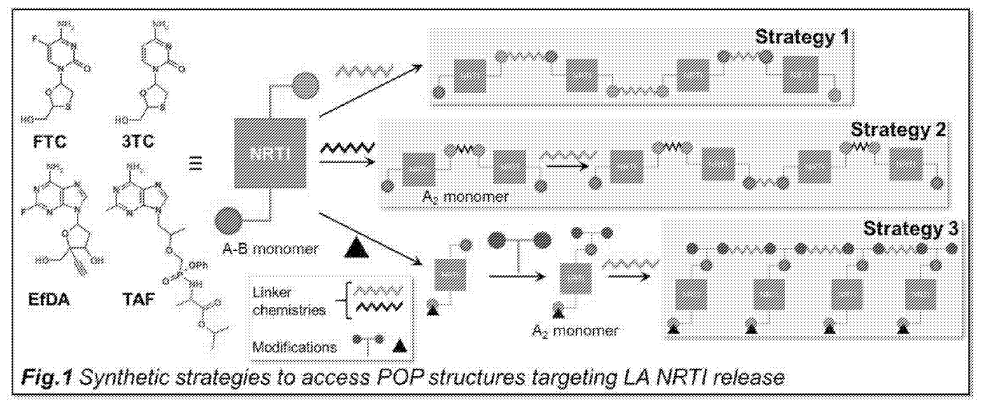

FIG. 1 shows NRTIs (TFV, FTC, 3TC and EFdA) which have multiple functional groups for step-growth polymerization strategies.

The present invention provides several new solutions including three synthetic strategies (FIG. 1) which exploit the reactivity of NRTIs in polymerization, e.g.: 1) reaction of A-B NRTI monomers with complementary linkers (Strategy 1); 2) reaction of A₂ prodrug monomers with B₂ linkers (Strategy 2); and 3) reaction of A₂ pendant prodrug monomers with B₂ linkers (Strategy 3).

Thus, a key innovative aspect of the present invention is the use of “Polymer-of-Prodrug” (POP) approaches that exploit NRTI prodrug strategies to address the critical need for LA NRTI regimens. These NRTI prodrug strategies enable synthesis of biodegradable NRTI POP structures. Materials with specific polymer structures are used to create polymer constructs which are physically assembled embodiments of the polymer structures (nanoparticles or macro-scale implants).

POP constructs offer considerable additional control of prodrug and drug release, initially from the construct and latterly from POP structure degradation. POP structure degradation generates a series of molecular species, or “POP fragments”, that comprise individual prodrug structures. Collectively, the POP fragments lead to an averaged release of parent NRTI, controlled by individual degradation rates within the mixture.

The present invention allows the preparation of polymers that degrade to prodrug monomers (POP approach), to deliver NRTIs at rates appropriate to match cabotegravir and rilpivirine dosing frequencies and enable development of complete LA regimens.

The NRTI prodrug scaffolds and POP structure degradation facilitate the application to intramuscular depots comprising POP nanoparticle suspensions or subcutaneously administered monolithic POP constructs.

POP Structures Using FTC and 3TC Prodrug Monomers

One category of products in accordance with the present invention comprises POP structures using FTC or 3TC prodrug monomers. The polymers may be inherently water-insoluble to allow generation of formats compatible with injection or implantation and long-acting release.

Conventional step-growth reactions using A-B monomers bearing orthogonal functional groups requires a second bifunctional compound to react with A and B functionalities. FTC and 3TC, containing amino and hydroxyl groups, are considered A-B monomers capable of reacting with carboxylic acid analogs (acid chlorides, chloroformates, etc.). This approach produces polymers comprised of prodrug monomers (FIG. 1; Strategy 1). Selectively masking NRTI functional groups will enable synthesis of novel A₂ monomers that can be used to generate polymers through reaction with a B₂ monomer, leading to step-growth polymers with pendant prodrug moieties (FIG. 1; Strategy 3). These strategies to access POP structures allow control of NRTI-prodrug and NRTI release, such that target release rates commensurate with desired LA clinical timescales are achievable.

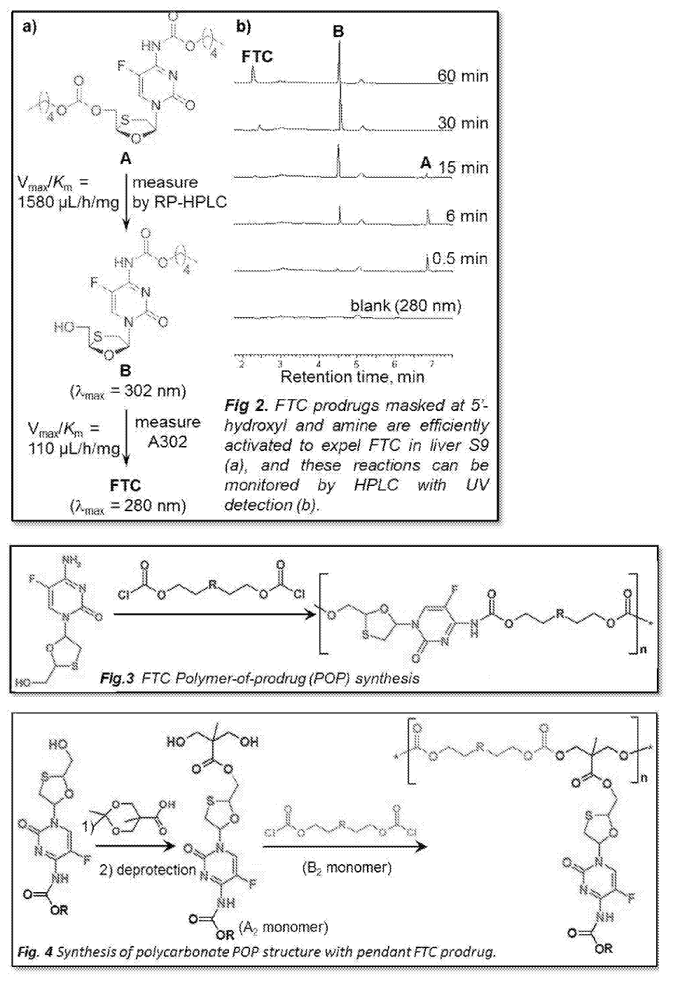

The polymer-of-prodrug approach allows LA dosing of water-soluble NRTIs. TFV is a clinically validated component of backbone therapies and may be used in combination with emtricitabine (FTC) and rilpivirine. LA NRTIs have clear clinical relevance to support rilpivirine LA formulations in development.^{21,22} Likewise, 3TC and EFdA (the most potent ARV discovered to date) are compatible witth LA approaches and are amenable to POP strategies. Our work has generated NRTI prodrugs (e.g. FTC, FIG. 2) for other LA formats. We have implemented reversible prodrug masking chemistry to enable SDN formation. One such approach can be used to mask the FTC amine and hydroxyl groups as carbamates/carbonates (A, FIG. 2)

POP Based on FTC and 3TC (Strategy 1)

Bioreversible masking of FTC and 3TC can be optimized to permit polymer synthesis and controlled NRTI release. The amine and hydroxyl groups on FTC and 3TC are readily converted to carbamate and carbonate groups using chloroformate reagents. We have exploited this reactivity to generate a scalable route to carbamate/carbonate prodrugs (FIG. 2a) with flexibility to control log P, molecular weight and hydrolysis rates, thus enabling tuning of parent drug release to target LA timescales. Bis-chloroformates and analogs (e.g. bis-carbonyl imidazoles) are readily available. FTC and 3TC are considered A-B monomers that require a single linker chemistry to form polymer structures (Strategy 1; FIG. 1). Reaction of bis-chloroformates with NRTIs produces polymer chains with a statistical arrangement of carbonate and carbamate groups along the backbone, resembling a repeating pattern of the prodrug molecules we have previously synthesized (FIG. 3). The exact sequence will depend upon reaction conditions. This stochastic arrangement (FIG. 1, Strategy 1) should not have deleterious effects on chain degradation and prodrug release. The linker choice will determine the molecular weight and overall drug density of the polymer chain, polymer hydrophilicity and drug release kinetics. Polymer degradation should release only a few POP fragment types. The fragments will ultimately degrade to generate diol and parent drug as the sole products. Optimized POP structures can support polymer degradation and NRTI release such that dosing intervals can be matched to rilpivirine and cabotegravir LA. Materials with a longer predicted dosing interval will be studied as potential candidates for Pre-exposure prophylaxis PrEP (where single agents have utility).

POP Using FTC and 3TC as Pendant Prodrug Monomers (Strategy 3):

Therapeutic polymer prodrugs typically incorporate drug moieties pendant to the polymer backbone.²⁹⁻³¹ The present invention generates diol-containing A₂ monomer prodrugs for polycarbonate step-growth synthesis (FIG. 4), using masking chemistry already established. A variety of B₂ monomers (e.g. bis-chloroformate linkers) are available for coupling to the diol, offering flexibility to tune the chemical and physical properties of POP structures. Polymers may release the NRTI prodrug or NRTI by various mechanisms arising from: a) prodrug cleavage from the main chain, b) cleavage of the main chain (prodrug release), or c) pendant carbamate cleavage and subsequent release of parent NRTI from the main chain. By varying B₂ linker and carbamate masking chemistry, we can control properties, degradation and hydrolysis kinetics of the POP structure. For example, we have shown that carbamate alkyl chain length dictates hydrolysis kinetics of NRTI carbamate prodrugs. Formation of the POP structure further extends NRTI release kinetics relative to small molecule systems. Linker chemistry may be optimized to increase hydrophilicity of the polymer backbone, to aid water penetration (e.g. use tri(ethylene glycol) bis(chloroformate) to introduce short polyethylene glycol chains).

POP Structures Based on Tenofovir Alafenamide and EFdA

One category of products in accordance with the present invention comprises biodegradable polymers that incorporate water-soluble NRTIs tenofovir or MK-8591 (EFdA). The polymers may be water-insoluble polymers and may be used in injectable or implantable constructs.

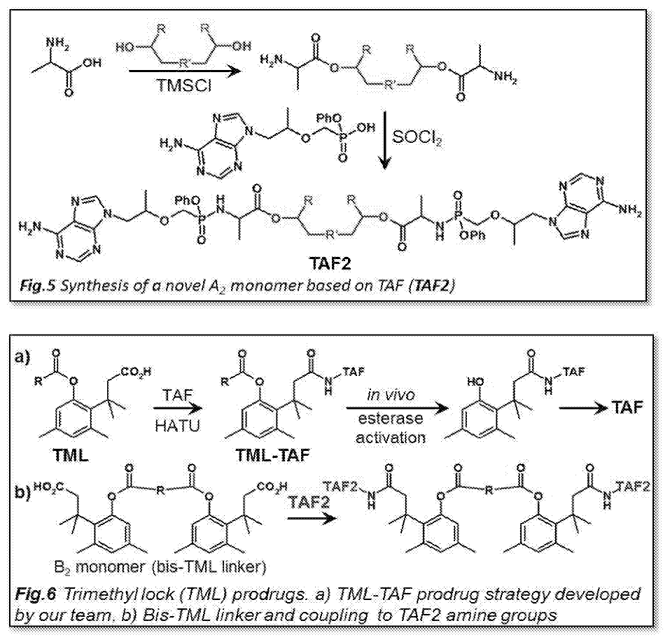

Clinically-used prodrugs for delivery of tenofovir (TFV) include tenofovir disoproxil fumarate (TDF) and tenofovir alafenamide (TAF), prodrugs that enhance PK properties (i.e. cellular uptake) by masking the negatively-charged phosphonyl group. These water-soluble prodrugs bear one amine group and are not useful for generating POP structures. Our underlying logic is that the approach to access the alanine ester moiety in TAF (FIG. 1) can be used to prepare novel bifunctional A₂ TAF-analog monomers (TAF2, FIG. 5) for A₂+B₂ step-growth polymerization (Strategy 2; FIG. 1). Delivery of TAF analogs is particularly appealing as TAF (and possibly its analogs) accumulates in HIV-infected cells prior to releasing TFV,³² suggesting lower doses could lead to lower depot volumes. EFdA is a potent new NRTI (in vitro IC₅₀˜0.2 nM) with predicted in vitro potency ˜8400-fold higher than TFV³³. Early EFdA primate PK studies suggest a plasma t_{1/2} of ˜7 hours (intracellular tin of triphosphate >72 h). Interestingly, viral suppression is maintained for a minimum of 7 days after the last dose, suggesting a once-weekly oral dosing option. The high potency and intracellular t_{1/2} make this NRTI ideal for LA formulation. Our rationale is that the amine and diol functional groups on EFdA render it ideal as an A₂ pendant prodrug monomer. POP structures based on TAF or EFdA prodrug monomers can degrade controllably to release NRTI prodrug analogs and parent NRTIs. The synthetic strategies impart flexibility for tuning polymer properties and drug release kinetics to match the dosing frequencies for rilpivirine and cabotegravir LA.

Synthesis of A₂ and B₂ monomers is central to this strategy. For TAF-based POP structures, we have methods in place to prepare modified alanine diesters from available diols (TAF2, FIG. 5). Generation of polymeric structures with TAF2 requires reaction with a bifunctional linker (B₂ monomer) under conventional step-growth polymerization conditions. B₂ monomers incorporating bloreversible, self-immolative trimethyl lock (TML) groups³′ amenable to coupling with the amino group of TAF2 may be used for the generation of POP structures (FIG. 6). Our work has accessed TML-TAF prodrugs susceptible to in vivo esterase activation (FIG. 6a). This approach is applicable to the use of novel bis-TML linkers (FIG. 6b) as bifunctional B₂ monomers for POP structure synthesis (Strategy 2; FIG. 1). The trifunctional EFdA scaffold enables us to explore several EFdA-based POP strategies. TAF prodrug chemistry developed by our team is applicable to an approach that combines features of Strategies 1 and 3 (FIG. 1).

POP Using TAF-Based Monomers (Strategy 2):

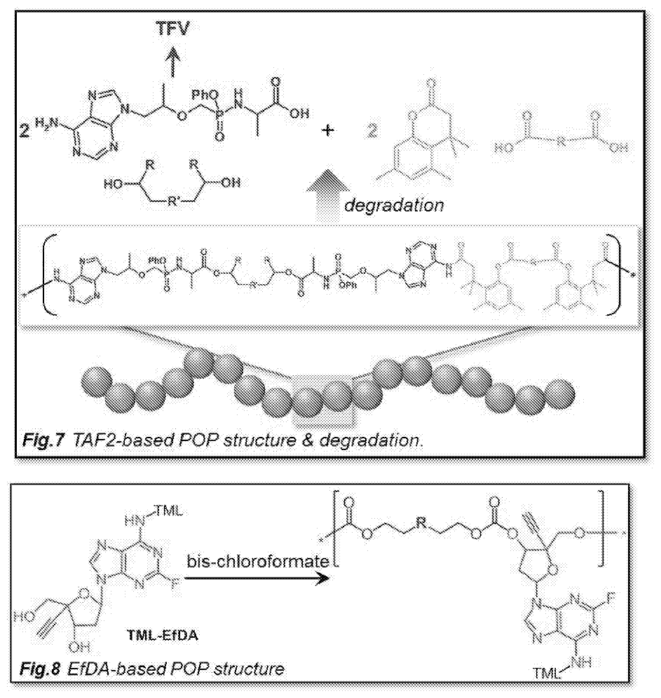

A₂ monomers may be prepared (TAF2, FIG. 5) using commercially available diols. B₂ monomers (bis-TML linker, FIG. 6) may be prepared by established routes to TML esters.³⁵ A₂ and B₂ monomers may be used directly for POP synthesis (FIG. 6b). POP structure degradation to release NRTI prodrug and NRTI is driven by esterase-mediated hydrolysis (FIG. 7), which may occur at the surface of an implanted monolith or suspended polymer nanoparticles. Hydrolysis of bis-TML linkers in the polymer can facilitate cyclization and release of the TAF amine. The present invention allows sufficiently long tin of POP structure and constructs, extending release to be compatible with rilpivirine and cabotegravir LA (or longer for PreP).

POP Derived from EFdA (Mixed Strategy 2/3):

EFdA bears an amine and two hydroxyl groups (FIG. 1); thus, a mixed POP synthesis strategy is effective. The amine group can be masked with TML as described for TAF, to give TML-EFdA (FIG. 8). The TML-EFdA diol can be coupled to a variety of bischloroformates to form linear step-growth polymers poised to degrade to EFdA or TML-EFdA prodrug. Again, the synthetic approach enables tuning of EFdA POP properties and control of hydrolysis rates. For example, varying the TML ester group alters the ester hydrolysis rate and amine release. Varying the linker impacts polymer backbone conformation and enzymatic access to hydrolysis sites.

POP Structure Degradation:

POP polymers degrade to form several molecular fragments under physiological conditions. During degradation, the drug and POP fragments are released into the muscle tissue and then enter the systemic circulation, being exposed to liver metabolism and possibly infected peripheral blood mononuclear cells (PBMCs).³ Hydrolysis rates of polymers are assessed under model physiological conditions (pH 7.4, 37° C.), in commercially available human plasma, muscle and liver S9 fractions, PBMC S9 fractions, and in commercially available buffer simulating the subcutaneous environment. Polymer degradation can be monitored by HPLC (detect fragments/NRTIs), size exclusion chromatography (detecting changes in molecular weight and distribution) and ¹H NMR (detecting changes in backbone signals). Appearance of NRTI and NRTI prodrug fragments can be monitored by HPLC.

In Vitro Antiviral Activity and Cytotoxicity.

Antiviral activity may be assessed by single-round infectivity assays as described.³⁷ POP structures are assessed for their ability to attenuate infection by preincubation with activated T cells prior to viral challenge. It was previously shown that the dose-response curve slope affects the instantaneous inhibitory potential, or log increase in the inhibition of infectivity, and that these slope values are specific for different ARV classes.³⁸ Regarding the mechanisms of action of POP structures and fragments and constructs, dose-response curves obtained from single-round infectivity assays are analyzed using the median effect equation to determine the slope, compared to parent drug.

In Vitro Drug Release Kinetics

NRTI release may be studied using NMR, UV, HPLC and LCMS to determine in vitro drug release kinetics. As noted, during drug release the NRTI and POP fragments are released into the tissue and will travel through tissue to reach the blood capillaries. The muscle interstitium consists of a collagen fiber framework containing a gel phase made of glycosaminoglycans, a salt solution, and plasma-derived proteins.^{39,40} Drug release rate from POP structures and constructs in buffer simulating the subcutaneous environment and the interstitial fluid can be tested using microdialysis. The influence of the POP construct type (nanoparticle or solid implant) and any additional excipients (e.g. gelators, polymers and surfactants) on release rate and stability may be investigated using linear and non-linear regression analysis. The release rate of NRTI and molecular fragments from the POP materials may be compared to optimal release rates calculated through PBPK modelling.

POP Implants and Nonparties Dispersions—POP Constructs.

Products in accordance with the present invention may take the form of injectable or implantable compositions. The products may be injectable polymer nanoparticle dispersions.

The water-solubility of NRTIs has previously negated their use in LA regimens. The NRTI LA formulations of the present invention facilitate the tuning of NRTI LA dosing intervals to, inter alia, that of commercial candidate LA technologies such as rilpivirine and cabotegravir LA. The present invention allows the manipulation of NRTI release from POP structures. The polymer of NRTI prodrug approach is advantageous, consistent with the growing evidence that polymer therapeutics and polymers-of-drug monomers are clinically viable. Long-acting ARV delivery may be achieved by several routes including two proven approaches.

1) Depot administration using aqueous dispersions of polymer nanoparticles with encapsulated drug. Nanoparticles of water-insoluble organic compounds are produced by nanoprecipitation on a large commercial scale for food applications. Such nanoprecipitation approaches are demonstrated to encapsulate water-insoluble drugs within degradable polymers (often poly(lactic-co-glycolic acid, PLGA). We have evaluated aqueous polymer nanoparticle dispersions and injectable gels comprised of POP structures, representing a highly novel approach to polymer therapeutics and nanomedicine.

2) Implant technologies permit cessation of dosing, if clinically required, by removal of the physical structure. This offers significant benefits but requires the formation of relatively large, solid, monolithic structures from POP materials. There are several approaches to forming monolithic structures from polymers, including melt processing and direct compaction, both of which are comprised here. For exemplification we selected FTC and a prodrug of TFV on the basis that the parent NRTIs are clinically validated as backbone therapies for rilpivirine and cabotegravir LA formulations.^{21,22} 3TC and EFdA also offer distinct benefits and LA formulations of these provide additional therapeutic options. Thus, NRTI LA platforms are compatible with LA products developed by Janssen and ViiV Healthcare, enabling development of two separate complete LA regimens and obviating the need to dose rilpivirine and cabotegravir LA concurrently (shown in the LATTE 2 study).

POD approaches are the basis of a start-up company “Polymer Therapeutics” (PRx, Rutgers Univ.) which has used salicylic acid and diflunisal as monomers to generate drug release polymers for short-duration drug delivery. As noted, such systems fully degrade during drug release avoiding the need for surgical removal. This technology was demonstrated in rodents and pigs, including measurement of in vivo PK of drug release over a wide range of doses for developmental PolySA™ and PolyDF™ platforms. These products do not show appreciable toxicity and, importantly, lack undesirable “burst release” behavior often observed from polymerencapsulated drug systems (e.g. PLGA nanoparticles). Thus, the principle for drug release from polymers synthesized directly from drug monomers is established.

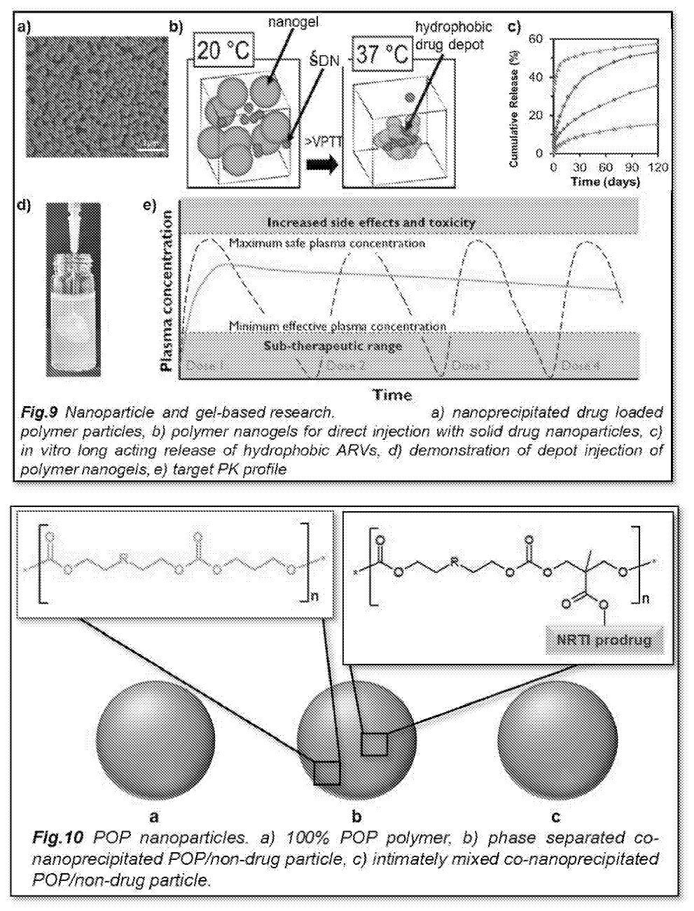

We have established the feasibility of NRTI prodrug monomer synthesis, and we have shown NRTI release kinetics to be tunable by modification of prodrug structures. Further, we have perfected nanoprecipitation techniques for a range of novel polymers⁴¹⁻⁴⁶ allowing the control of nanoparticle diameter, degradation, surface chemistry and zeta potential within highly stable (>2 years) aqueous dispersions (FIG. 9a). Additional studies have focused on using ARV drug nanoparticles in polymeric nanogels (FIG. 9b) to establish controllable delayed drug release over several months (FIG. 9c). Nanogels are injectable (FIG. 9d) and target ideal drug plasma concentrations that are maintained above oral-dose derived Cₘᵢₙ values, thereby offering options for LA technologies (FIG. 9e).

POP Nanoparticle Dispersions and Gels

The POP polymers behave like other polymers (e.g. polycarbonates and polyesters) shown to undergo nanoprecipitation into aqueous media. This process involves creating a polymer solution in a water-miscible organic solvent and addition to an aqueous medium in which rapid dilution of the organic solvent leads to polymer precipitation. The presence of stabilizers during nanoprecipitation influences particle diameters and zeta potentials; other variables include solvent choice, dilution (solvent to precipitant ratio) and temperature. Conventional drug encapsulation using hydrophobic polymers can undergo variable 3-stage drug release with a “burst release” initially after injection, followed by a controlled, linear release and a subsequent final “burst” during polymer degradation. Attaining zero-order, or pseudo-zero order, release kinetics is ideal to maintain NRTI plasma concentration. Degradation of nanoparticles derived from POP materials may be more controlled and approximate zero-order kinetics due to drug release being intrinsically linked to the physical degradation of the nanoparticles. This principle is demonstrated for POD materials and hydrogel implants containing paclitaxel-loaded PLGA microspheres showing near zero-order release for >60 days.⁴⁷ Co-nanoprecipitation (FIG. 10) was pioneered by our team; release kinetics can be modified by blending of analogous degradable, non-drug based polymers, if required. Here, the influence of nanoparticle properties, polymer chemistry, presence of co-nanoprecipitated polymers and the aqueous environment allow the optimization of NRTI and prodrug release. In addition, small molecule gelators (e.g. peptides) may be used to thicken or gel the aqueous depot site, offering additional parameters to moderate release. NRTI POP nanoparticle combinations are readily achieved by mixing nanoprecipitates before injection and matching release timescales and doses. Multiple implants containing different drugs may also be administered (established with contraceptive implants) allowing personalized treatments and options to overcome drug-drug interactions.

Solid State Compaction of POP Materials:

Cold compaction of powdered polymers to form molded products has been investigated^{48,49} to generate relatively small objects. Typically, a simple die is used to compress the powder at ambient temperature, or slightly above the glass transition temperature, and small discs or rods may be readily formed. Later thermal treatment may be required to improve the physical properties of the compacted structures. Small implants that degrade to release entrapped drugs were developed using similar techniques, including small formulated solid rods (by Glide Technologies⁵⁰⁻⁵²) comprising drug and excipient (e.g. sugars and polymers) that are administered subcutaneously using a novel needle-free actuator. POP materials of the present invention have been compacted individually, in combination or with analogous non-drug based polymers. Unlike conventional implants, POP constructs completely degrade leaving minimal residual solid at the administration site. Physical implants with zero/pseudo-zero order drug release over several months are well-described,⁵³ and recent molded structures have been produced after mixing a small molecule (carmustine) with PLGA in solution and drying to a fine powder before compaction. In this case a controlled near-zero order release was obtained for 4 weeks.⁵⁴ The POP constructs of the present invention may be modified to control porosity or compacted with rapidly dissolving excipients (e.g. sugars) to generate porosity after administration. Zero-order, or near zero-order, release kinetics may be obtained over desired timescales.

Pharmacological and Toxicological Analyses of POP Structures and Constructs

Cellular accumulation and cytotoxicity of POP components (assessing potential augmented cellular prodrug delivery in the absence of overt cellular toxicity) is important and POP constructs with clinically relevant release rates have been modelled to predict preclinical and human doses using PBPK models^{55,56}; release rates of approximately 0.0046 h⁻¹ are known to provide appropriate duration of drug exposure. Ideally modelling uses experimental in vitro data describing tissue distribution and clearance and establishes simulations for 100 individuals using Simbiology, MATLAB, R2013b. Important mathematical descriptions include covariance between demographics and tissue size, expression of metabolic enzymes and processes regulating absorption, distribution and elimination, which are drug-specific, validated against real clinical data for orally administered standard formulations. NRTI dose and release rate are estimated to allow prediction of C_{trough} values above the IC₉₅ for each drug. TFV and FTC are particularly good candidates for monthly depot formats (or longer).⁵⁵ For example, and based on the known clearance of these drugs, the models estimate that monthly exposure above the IC₉₅ for each drug could be achieved from doses of 1500 mg and 600 mg, and release rates of 0.0015 h⁻¹ and 0.001 h⁻¹ for TFV and FTC, respectively. These data have been used to develop POP structures and constructs.

Pharmacology of POP Structures:

It may be the case that POP structures (implant or POP nanoparticles) may satisfy the following pharmacological criteria: 1) higher hydrolysis rate at pH 7.4 than parent NRTI clearance rates, 2) POP fragments that exhibit anti-infective activity by the parent NRTI mechanism, 3) no adverse safety or toxicity concerns, and 5) antiviral IC₅₀<parent NRTIs.

Cytotoxicity: Cytotoxicity is assessed in primary human CD4+, CD8+ and CD56+(natural killer) cells, platelets, red blood cells, monocytes, monocyte-derived macrophages, monocyte-derved dendritic cells, primary hepatocytes, muscle cells and adipocytes. These are chosen for site of action/administration, safety and presence of drug metabolism to assess potentially toxic POP fragments. Assays target membrane integrity (trypan blue), mitochondrial function (MTT) and oxidative stress/lipid peroxidation (GSH/GSSG ratio).

Cellular accumulation: Development of TAF as an alternative to TDF has shown a benefit in terms of dose and safety for TFV delivery; thus, our tests determine whether POP fragments accumulate in target cells. This facilitates identification of materials able to deliver higher free drug concentrations intracellularly. For this, POP fragments are incubated with freshly isolated PBMCs at concentrations relevant to those achieved for the existing clinically used drugs. Subsequently, the intracellular concentrations are determined by scintillation counting (or traditional bioanalytical method if needed) and expressed as a cellular accumulation ratio (CAR) relative to extracellular concentration measurements.

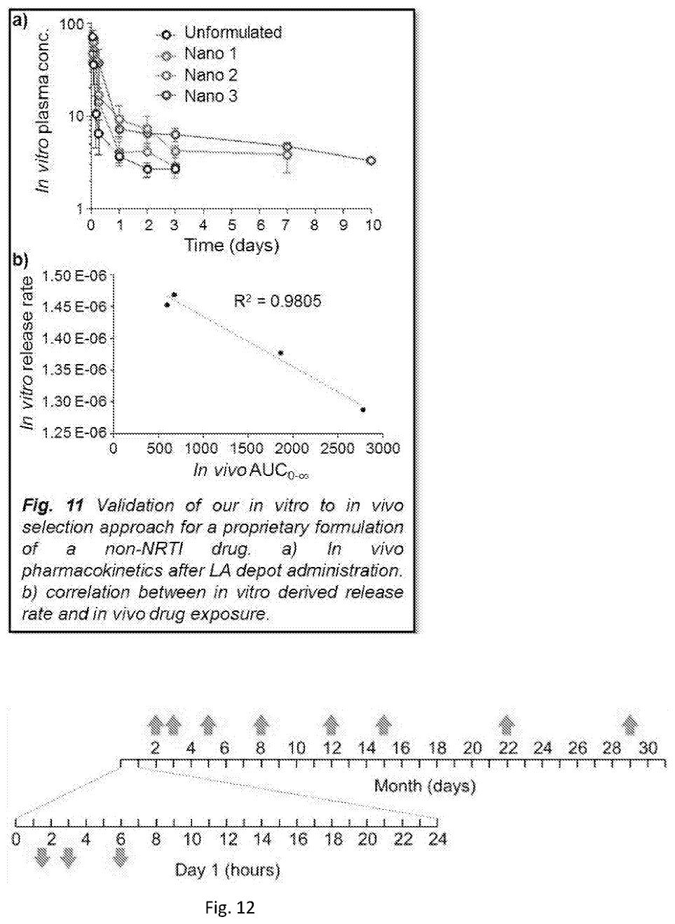

Detailed pharmacological evaluation of POP constructs. POP constructs with established stability and in vitro drug release kinetics progress to detailed ex vivo and in vivo evaluation and subsequent PBPK modelling. Our expertise for in vitro release rate selection progressing to in vivo confirmation of behavior is exemplified by recent work with a proprietary formulation (FIG. 11).

Ex vivo analysis of drug release in porcine tissue: The feasibility of sustained release from a POP construct is investigated in porcine tissue, quantifying release rate from tissue using a modified Franz diffusion cell model. This experimental method allows clamping of a section of porcine tissue with subsequent application of a controlled flow of fluid, simulating blood flow. POP constructs are injected or implanted into the tissue at controlled depths and the NRTI release rate into the donor compartment is measured over time. Porcine tissue is used in these experiments because porcine soft tissues are very similar to human soft tissue in terms of morphology and function. In some cases, experiments use radiolabeled and/or unlabeled POP constructs and comparisons relative to aqueous solutions as controls are studied. The impact of release on POP implant integrity is assessed over the period of release to establish the potential for removal during dosing. Likewise, POP nanoparticles are studied to assess the potential for transit of intact particles within tissues. This is assessed by FRET fluorescence studies, through encapsulation of FRET pairs within the POP nanoprecipitates, and by flow cytometry methods we have developed. Parent NRTI concentrations are quantified by scintillation counting (where applicable) or validated bioanalytical methods.

Pharmacoldnetcs and tissue distribution in preclinkcal species: Lead POP constructs are studied initially in Wistar rats with confirmation in rabbits for those showing promise. This approach was developed due to known species differences in enzymes involved in prodrug activation.⁶⁵ Specifically, for some hydrolases rodents are more appropriate but for others rabbits are, and we use both to get better coverage of hydrolase activities. Three or four dosages of each POP construct are studied, after performing initial tolerability studies. A minimum of 3 animals are used for each dose. Animals in each dosing cohort are sampled from plasma according to the sampling strategy shown in FIG. 12 (arrows represent time of blood withdrawal from the tail vein; three samples taken on the first day at 1.5, 3 and 6 hours, followed by a single plasma sample on days 2, 3, 5, 8, 12, 15, 22 and 29). We use equal numbers of male and female animals to gauge potential differences in responses related to sex, and we perform the studies blinded to drug treatment to avoid bias. A lack of pre-existing depot formulations prohibits comparative testing of emerging formulations; thus, blinded comparisons are not possible. Quantitative predictions of exposure in humans are developed by PBPK modeling, and the in vivo experimental outcomes will essentially be qualitative in terms of the ability to provide LA exposure. Animals are sacrificed when concentrations fall below detectable concentrations (using CO₂ asphyxia) irrespective of time post dose. Following sacrifice, a physical examination of implant site is conducted to establish local responses to POP constructs. A histological examination of the implant site is also conducted as part of our safety assessment. Tissues (including brain, lymph nodes, liver, kidneys, lungs, etc., necessary for a robust assessment of overall distribution) are removed, and stored at −80° C. until further analysis. Analyte concentrations in plasma and brain are measured using bioanalytical methods or detection of an incorporated radiolabel. Additionally, plasma concentrations of proinflammatory cytokines (IL-1b, IL-6, IL-8, TNFα and IFNγ) as well as markers of inflammation (HMGB1, CRP, and fibrinogen) and platelet activation (P-selectin, sICAM-3) are also assessed. The PK is described using compartmental modeling techniques. The AUC in the respective compartments and the extent of penetration into tissues is estimated. It remains unclear with existing sustained release formulations (rilpivirine LA and cabotegravir LA) whether following administration any SDNs enter the systemic circulation as intact nanoparticles; POP nanoprecipitates also have this potential. These are assessed using validated flow cytometry protocols which also enable study of protein corona that may influence biodistribution⁶⁶ and immunological safety⁶⁷ of nanoparticles entering the systemic circulation. Preclinical data in rodents generated for rilpivirine LA are used to benchmark success in these experiments.

In vitro safety assessment A preliminary nanotoxicology assessment, including intensive immunological analyses, is conducted. This includes analysis of POP structures, precursors, drug/prodrug and fragments that are biocompatible. As FTC is a nucleoside analogue there is a possibility that FTC POP (or their fragments) may be immunogenic (nucleosides are ligands for TLR 7/8, and it is possible that other nucleic acid-like structures may also be ligands for pattern recognition receptors therefore warranting investigation^{68,69}). In addition to their composition it is possible that the “fiber-like” nature of the POP may prevent the complete engulfment of these materials by macrophages and result in frustrated phagocytosis hallmarked by a burst of macrophage-released proinflammatory mediators.⁷⁰ These possibilities support the inclusion of both “final product” POPs as well as precursors and POP fragments. A tiered approach to biocompatibility testing is as follows.

Tier I: sterility testing of materials to rule out any potential false positives in subsequent immunological assays. Solutions containing the materials screened for microbial contamination. The presence of endotoxin assessed using chromogenic or turbidimetric versions of the Limulus Amoebocyte Lysate (LAL) assay. If particles were found to be ‘clean’, progress to the second tier of the analysis.

Tier II: assessment of common acute toxicities, including haemolysis (RBC destruction), complement activation, thrombogenicity, induction of pro-inflammatory cytokines (primarily IL-1β, IL-8 and TNFα), leukocyte proliferation (³H-thymidine incorporation), uptake of POPs by macrophages and neutrophils.

Tier III: impact of materials on immune cells and their functions. Effects on macrophage function assessed by measuring phagocytosis, cytokine secretion and immunophenotyping, and effects on neutrophil function by monitoring cytokine secretion, generation of oxidative burst and neutrophil extracellular traps. Determine how POP uptake effects antigen and mitogen induced leukocyte proliferation in immune cells, examine if POPs affect natural killer cell cytotoxicity, determine if POPs affect dendritic cell maturation and examine the impact on cytotoxic T-lymphocyte activity. Immunophenotyping in whole blood may also be implemented to determine if interactions with immune cells affect immune cell phenotype. If any interactions are observed, follow this up in Tier IV with mechanistic assays, such as determining transcription factor activation, caspase activation, cellular health assays, mechanisms of cell death (immunologically silent/not silent) and receptor inhibition studies. Data benchmarked against literature and our existing data generated through our participation in the European Nanomedicine Characterization Laboratory (www.euncl.eu).

In Vitro-In Vivo Extrapolation (IVIVE) Using PBPK Modeling:

As outlined above, PBPK modeling is widely used by the pharmaceutical industry and we recently developed robust models for a number of ARVs in an open source environment.^{55,60,62,71-73} This has led to the generation of the first PBPK models to simulate the PK of LA formulations, identifying optimal doses and release rates for sustained exposure after intramuscular depot injection. This is a uniquely powerful tool for evaluating POP constructs and the program described here enables validation of a range of assumptions within such predictive models.

### EXPERIMENTAL DATA AND INFORMATION

### Synthesis and Characterisation of Pop Materials

Nuclear magnetic resonance (NMR) spectra were recorded using Bruker Avance III 400 MHz and 500 MHz spectrometers. Chemical shifts (6) are reported in parts per million (ppm) relative to tetramethylsilane (TMS) internal standard for both ¹H and ¹³C spectra.

Molecular weights of polymers were characterised by gel permeation chromatography (GPC) performed in dimethylformamide (DMF) containing 0.01 M LiBr at 60° C., with a flow rate of 1 mL min⁻¹ using a Malvem Viscotek GPCmax instrument equipped with two Viscotek T6000 columns, a refractive index detector (RID) VE3580 and a 270 Dual Detector (light scattering and viscometer) or an Agilent 1260 Infinity II instrument equipped with a RID and PLGel column (3 μm Mixed-E).

Electrospray ionisation mass spectrometry (ESI-MS) data were obtained using an Agilent QTOF 6540 mass spectrometer using positive electron ionisation and direct infusion syringe pump sampling.

POP Structures Using FTC and 3TC Prodrug Monomers

### Synthesis and Characterisation of NRTI Polymer-of-Prodrug—bis(chloroformate) Route

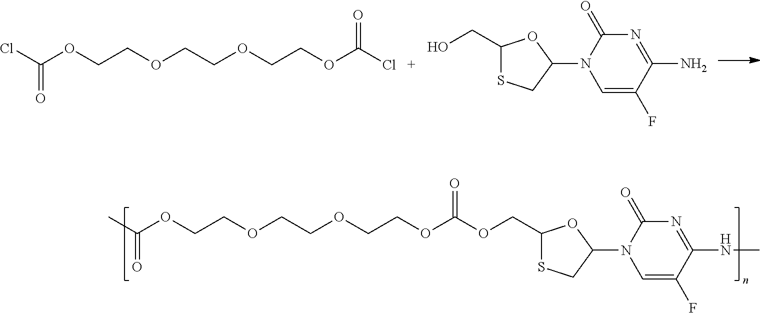

### FTC Polymer-of-Prodrug (POP) Synthesis with bis(chloroformate)

General synthesis of linear FTC POP structure using bis(chloroformate), poly[(triethylene glycol)/FTC]: To a dry 10 mL round-bottomed flask containing emtricitabine (FTC) (4.5 g, 18.2 mmol), 4-dimethylaminopyridine (DMAP) (1.11 g, 9.10 mmol) and pyridine (3.23 mL, 40.0 mmol) was added an anhydrous dichloromethane solution (7.15 mL, 50 wt %) of tri(ethylene glycol) bis(chloroformate) (3.74 mL, 18.2 mmol) dropwise with stirring at 0° C. under a nitrogen atmosphere over 30 min. The mixture was allowed to warm to ambient temperature and stirred for 16 hours to give a yellow viscous solution. The crude reaction mixture was dissolved in dichloromethane (300 mL) and washed with hydrochloric acid (1M, 2×150 mL) and saturated sodium chloride solution (3×150 mL). The organic layer was dried over magnesium sulphate, filtered and concentrated in vacuo to yield a colourless crispy solid. ¹H NMR (400 MHz, CDCl₃) δ ppm 3.24-3.27 (d, J=12.49 Hz, 1H), 3.55-3.60 (dd, J=5.11, 12.49 Hz, 1H), 3.63-3.67 (m, 4H), 3.71-3.77 (m, 4H), 4.30-4.36 (m, 4H), 4.55-4.59 (m, 2H), 5.39-5.41 (m, 1H), 6.27-6.31 (m, 1H), 8.03-8.11 (m, 1H). ¹³C NMR (100 MHz, CDCl₃) δ ppm 38.27, 65.37, 66.68, 67.74, 68.73, 68.91, 70.54, 70.63, 84.13, 87.25, 126.32, 153.34, 153.50, 154.73.

TABLE 1
GPC data for FTC POP structures

| Experiment | | Monomer | | | GPC (DMF)^{a} | | | |
|---|---|---|---|---|---|---|---|---|
| no. | Monomer A | B | A:B | Solvent | Mₙ | Mw | Ð | DPₙ |
| 1 | tri(ethylene glycol) | FTC | 1.08:1 | Tetrahydro- | 2650 | 3330 | 1.254 | 6 |
| | bis(chloroformate) | | | furan | | | | |
| 2 | tri(ethylene glycol) | FTC | 1.08:1 | ethyl | 2450 | 2920 | 1.194 | 5 |
| | bis(chloroformate) | | | acetate | | | | |
| 3 | tri(ethylene glycol) | FTC | 1.08:1 | Dichloro- | 4790 | 6700 | 1.398 | 11 |
| | bis(chloroformate) | | | methane | | | | |
| 4 | tri(ethylene glycol) | FTC | 1:1 | Dichloro- | 5880 | 8530 | 1.451 | 12 |
| | bis(chloroformate) | | | methane | | | | |
| 5 | tri(ethylene glycol) | FTC | 0.95:1 | Dichloro- | 5020 | 7170 | 1.429 | 11 |
| | bis(chloroformate) | | | methane | | | | |
| 6 | tri(ethylene glycol) | FTC | 0.9:1 | Dichloro- | 4750 | 6630 | 1.397 | 10 |
| | bis(chloroformate) | | | methane | | | | |
| 7 | tri(ethylene glycol) | FTC | 1:1 | chloroform | 6770 | 8010 | 1.184 | 15 |
| | bis(chloroformate) | | | | | | | |

^{a}DMF containing 0.01M LiBr at 60° C., 1 mL min⁻¹ flow rate.
Mₙ—number average molecular weight,
M_{w}—weight average molecular weight,
Ð—dispersity,
DPₙ—number average degree of polymerisation

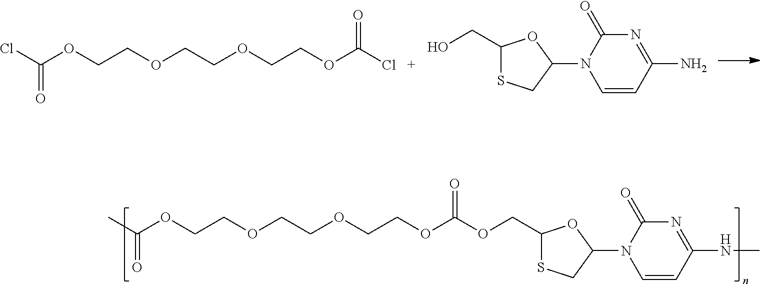

3TC Polymer-of-Prodrug (POP) Synthesis with Bis(Chloroformate)

General synthesis of linear 3TC POP structure using bis(chloroformate), poly[(triethylene glycol)/3TC]: To a dry 10 mL round-bottomed flask containing lamivudine (3TC) (4.5 g, 19.6 mmol), 4-dimethylaminopyridine (DMAP) (1.20 g, 9.81 mmol) and pyridine (3.48 mL, 43.2 mmol) was added an anhydrous dichloromethane solution (7.44 mL, 50 wt %) of tri(ethylene glycol) bis(chloroformate) (4.03 mL, 19.6 mmol) dropwise with stirring at 0° C. under a nitrogen atmosphere over 30 min. The mixture was allowed to warm to ambient temperature and stirred for 16 hours to give a yellow viscous solution. The crude reaction mixture was dissolved in dichloromethane (300 mL) and washed with hydrochloric acid (1M, 2×150 mL) and saturated sodium chloride solution (3×150 mL). The organic layer was dried over magnesium sulphate, filtered and concentrated in vacuo to yield a colourless crispy solid. ¹H NMR (400 MHz, CDCl₃) δ ppm 3.20-3.25 (m, 1H), 3.62-3.68 (m, 5H), 3.72-3.77 (m, 4H), 4.32-4.36 (m, 4H), 4.52-4.66 (m, 2H), 5.41-5.45 (m, 1H), 6.33-6.37 (m, 1H), 8.12-8.22 (m, 1H). ¹³C NMR (100 MHz, CDCl₃) δ ppm 38.69, 65.33, 67.09, 67.64, 68.81, 68.84, 68.93, 69.02, 70.64, 70.79, 84.09, 88.08, 94.92, 144.36, 152.52, 154.77, 154.83, 162.78.

TABLE 2
GPC data for 3TC POP structures

| Experiment | | Monomer | | | GPC (DMF)^{a} | | | |
|---|---|---|---|---|---|---|---|---|
| no. | Monomer A | B | A:B | Solvent | Mₙ | M_{w} | Ð | DPₙ |
| 8 | tri(ethylene glycol) | 3TC | 1.1:1 | dichloromethane | 5930 | 8500 | 1.432 | 14 |
| | bis(chloroformate) | | | | | | | |
| 9 | tri(ethylene glycol) | 3TC | 1:1 | dichloromethane | 5350 | 7560 | 1.415 | 13 |
| | bis(chloroformate) | | | | | | | |
| 10 | tri(ethylene glycol) | 3TC | 0.9:1 | dichloromethane | 5200 | 6780 | 1.302 | 12 |
| | bis(chloroformate) | | | | | | | |

^{a}DMF containing 0.01M LiBr at 60° C., 1 mL min⁻¹ flow rate.
Mₙ—number average molecular weight,
M_{w}—weight average molecular weight,
Ð—dispersity,
DPₙ—number average degree of polymerisation

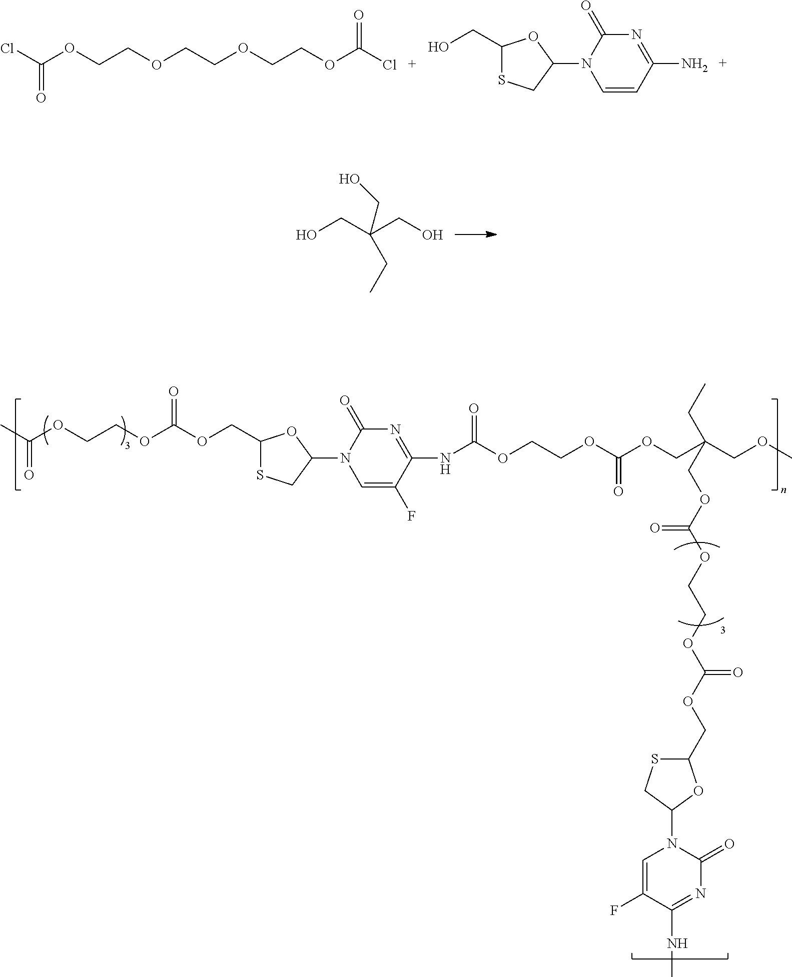

Branched FTC Polymer-of-Prodrug (POP) Synthesis with Bis(Chloroformate)

General synthesis of branched FTC POP structure using bis(chloroformate), poly[(triethylene glycol)/FTC/TMP]: To a dry 10 mL round-bottomed flask containing emtricitabine (FTC) (1 g, 4.04 mmol), trimethylolpropane (TMP) (0.040 g, 0.3 mmol), 4-dimethylaminopyridine (DMAP) (0.275 g, 2.25 mmol) and pyridine (0.80 mL, 9.89 mmol) was added an anhydrous dichloromethane solution (1.71 mL, 50 wt %) of tri(ethylene glycol) bis(chloroformate) (0.92 mL, 4.49 mmol) dropwise with stirring at 0° C. under a nitrogen atmosphere over 30 min. The mixture was allowed to warm to ambient temperature and stirred for 16 hours to give a yellow viscous solution. The crude reaction mixture was dissolved in dichloromethane (100 mL) and washed with hydrochloric acid (1M, 2×50 mL) and saturated sodium chloride solution (3×50 mL). The organic layer was dried over magnesium sulphate, filtered and concentrated in vacuo to yield a colourless crispy solid. ¹H NMR (400 MHz, CDCl₃) δ ppm 0.85-0.93 (m, 3H), 1.40-1.56 (m, 2H), 3.22-3.26 (d, J=12.42 Hz, 1H), 3.55-3.60 (dd, J=5.11, 12.42 Hz, 1H), 3.63-3.67 (m, 4H), 3.70-3.77 (m, 4H), 4.10 (s, 6H), 4.30-4.36 (m, 4H), 4.55-4.59 (m, 2H), 5.39-5.41 (m, 1H), 6.27-6.31 (m, 1H), 8.03-8.11 (m, 1H). ¹³C NMR (100 MHz, CDCl₃) δ ppm 7.29, 22.27, 38.26, 42.81, 65.38, 66.68, 67.04, 67.22, 67.72, 68.73, 68.92, 70.55, 70.61, 84.13, 87.31, 126.38, 140.39, 153.33, 153.50, 154.73, 155.13, 157.82, 158.09.

TABLE 3
GPC data for FTC branched POP structures

| Experiment | Monomer | Monomer | Monomer | | | GPC (DMF)^{a} | | | |
|---|---|---|---|---|---|---|---|---|---|
| no. | A | B | C | A:B | Solvent | Mₙ | M_{w} | Ð | DPₙ |
| 11 | tri(ethylene | FTC | TMP | 1:0.75: | Dichloro- | 10550 | 30600 | 2.901 | 23 |
| | glycol) | | | 0.25 | methane | | | | |
| | bis(chloro | | | | | | | | |
| | formate) | | | | | | | | |
| 12 | tri(ethylene | FTC | TMP | 1:0.9: | Dichloro- | 9290 | 13060 | 1.405 | 21 |
| | glycol) | | | 0.066 | methane | | | | |
| | bis(chloro | | | | | | | | |
| | formate) | | | | | | | | |

^{a}DMF containing 0.01M LiBr at 60° C., 1 mL min⁻¹ flow rate.
Mₙ—number average molecular weight,
M_{w}—weight average molecular weight,
Ð—dispersity,
DPₙ—number average degree of polymerisation

### Synthesis and Characterisation of NRTI Polymer-of-Prodrug—CDI Route

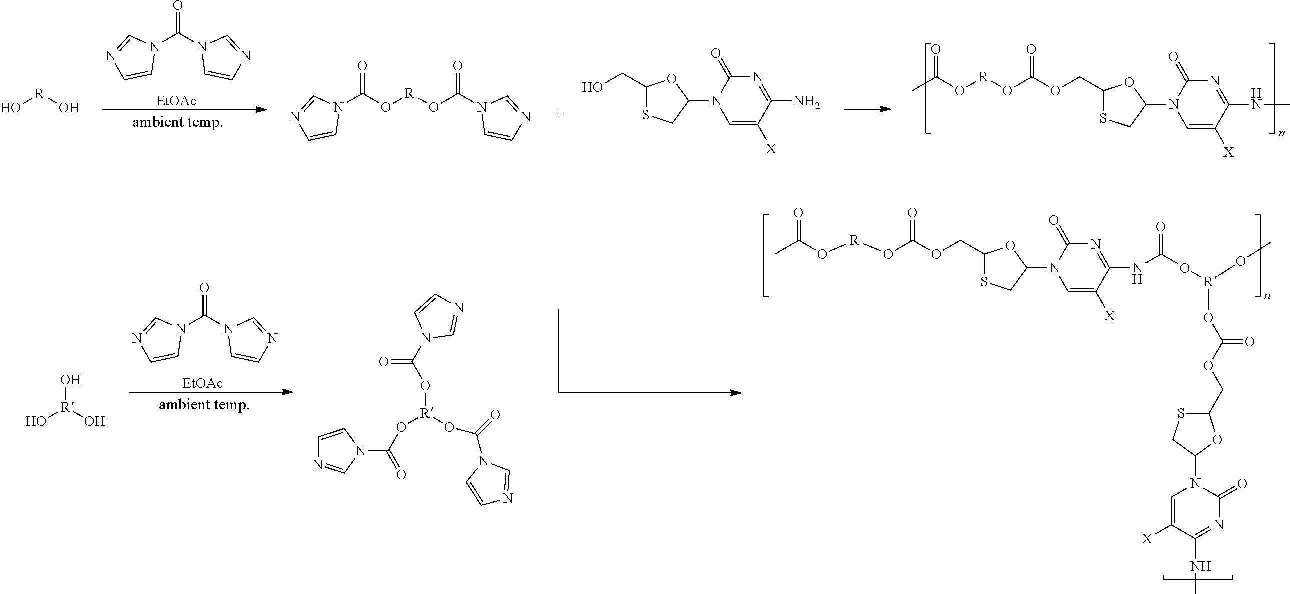 [0100] R and R′=an aromatic or aliphatic hydrocarbon chain (optionally≠C₁)
X═F or H

Overall Synthesis of NRTI POP Structures Using CDI Route

### Synthesis of CDI Activated Monomers

The synthesis of CDI-activated materials was carried out as outlined in literature.^{101,102}

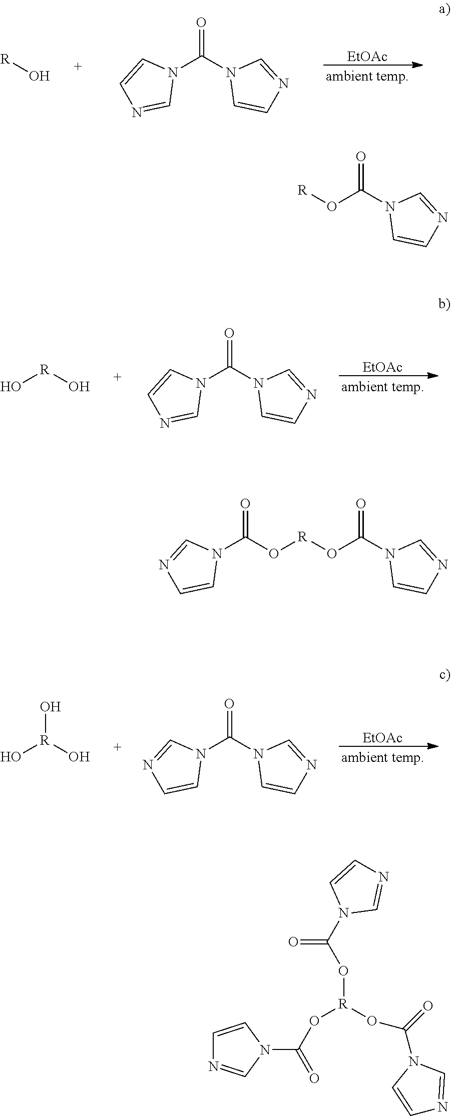

R=an aromatic or aliphatic hydrocarbon chain (optionally≠C₁)

### Synthesis of (a) imidazole carboxylic ester, (b) bis(imidazole carboxylic ester) and (c) tri(imidazole carboxylic ester)

Whilst exemplification herein primarily relates to CDI chemistry, in general the skilled person will understand that other activation chemistry may be used, for example triazole or other chemistry.

General synthesis of Imidazole carboxylic ester, dodecyl 1H-imidazole-1-carboxylate (active dodecanol): To a dry 250 mL round-bottomed flask containing dodecanol (3.73 g, mmol) and 1,1′-carbonyldiimidazole (CDI) (7.30 g, 45 mmol) was added anhydrous ethyl acetate (EtOAc) (60 mL) under a nitrogen atmosphere. The mixture was stirred at ambient temperature for 4 hours to give a yellow solution. The crude reaction mixture was dissolved in ethyl acetate (140 mL) and washed with deionised water (6×100 mL) and saturated sodium chloride solution (2×100 mL). The organic layer was dried over magnesium sulphate, filtered and concentrated in vacuo to yield a white solid, 5.14 g (89%). ¹H NMR (500 MHz, CDCl₃) δ ppm (t, J=7.74 Hz, 3H), 1.23 (br. m, 18H), 1.75 (m, 2H), 4.37 (t, J=6.89 Hz, 2H), 7.02 (s, 1H), 7.38 (s, 1H), 8.09 (s, 1H). ¹³C NMR (100 MHz, CDCl₃) δ ppm 14.03, 22.62, 25.66, 28.42, 29.09-20.55, 31.84, 68.43, 117.00, 130.52, 137.00, 148.69. Calcd: [M+H]⁺=281.4; found 281.2.

General synthesis of bis(imidazole carboxylic ester), hexane-1,6-dlyl bis(1H-imidazole-1-carboxylate) (active 1,6-hexanediol): To a dry 500 mL round-bottomed flask containing hexanediol (25 g, 0.21 mol) and 1,1′-carbonyldiimidazole (CDI) (77 g, 0.48 mol) was added anhydrous ethyl acetate (EtOAc) (200 mL) under a nitrogen atmosphere. The mixture was stirred at ambient temperature for 4 hours to give a yellow solution. The crude reaction mixture was dissolved in ethyl acetate (140 mL) and washed with deionised water (6×100 mL) and saturated sodium chloride solution (2×100 mL). The organic layer was dried over magnesium sulphate, filtered and concentrated in vacuo to yield a white solid, 47.4 g (72%). ¹H NMR (500 MHz, CDCl₃) δ ppm 1.53 (br. m, 4H), 1.84 (br. m, 4H), 4.44 (t, J=6.60 Hz, 4H), 7.08 (s, 2H), 7.43 (s, 2H), 8.14 (s, 2H). ¹³C NMR (100 MHz, CDCl₃) δ ppm 25.36, 28.40, 68.05, 117.06, 130.70, 137.06, 148.72. Calcd: [M+Na]⁺=329.3; found 329.1.

Ethane-1,2-dlylbis(oxy))bis(ethane-2,1-dlyl) bis(1H-imidazole-1-carboxylate) (active tri(ethyleneglycol)): Yield=0.357 g, (16%) ¹H NMR (400 MHz, CDCl₃) δ ppm 8.08 (t, J=1.0 Hz, 2H), 7.37 (t, J=1.5 Hz, 2H), 6.99 (dd, J=1.6, 0.8 Hz, 2H), 4.55-4.42 (m, 4H), 3.83-3.70 (m, 5H), 3.62 (s, 4H). ¹³C NMR (100 MHz, CDCl₃) δ ppm 148.62, 137.12, 130.64, 117.11, 77.47, 77.16, 76.84, 70.66, 68.64, 66.95.

General synthesis of tri(imidazole carboxylic ester), 2-(((1H-imidazole-1-carbonyl)oxy)methyl)-2-ethylpropane-1,3-diyl bis(1H-imidazole-1-carboxylate) bis(1H-Imidazole-1-carboxylate) (active TMP): To a dry 250 mL round-bottomed flask containing trimethylolpropane (TMP) (2.41 g, 18.0 mmol) and anhydrous ethyl acetate (EtOAc) (100 mL) was added 1,1′-carbonyldiimidazole (CDI) (14.6 g, 89.8 mmol) under a nitrogen atmosphere. The mixture was stirred at ambient temperature for 19 hours to give a yellow solution. The crude reaction mixture was washed with deionised water (4×50 mL) and saturated sodium chloride solution (1×50 mL). The organic layer was dried over magnesium sulphate, filtered and concentrated in vacuo to yield a white solid, 4.57 g (63%). ¹H NMR (400 MHz, CDCl₃) δ ppm 0.95-0.99 (t, J=7.59 Hz, 3H), 1.05-1.09 (dt, J=7.59 Hz, 3H), 1.52-1.60 (q, J=7.59 Hz, 2H), 1.67-1.73 (q, J=7.59 Hz, 2H), 4.05-4.32 (dq, J=7.59 Hz, 1H), 4.51 (s, 6H), 7.05 (s, 3H), 7.33 (s, 3H), 8.08 (s, 3H). ¹³C NMR (100 MHz, CDCl₃) δ ppm 7.47, 23.3, 42.1, 66.7, 116.9, 131.3, 136.9, 148.2. Calcd: [M+H⁺]=416.4; found 417.2.

Ethane-1,1,1-triyltris(benzene-4,1-dlyl) tris(1H-imidazole-1-carboxylate) (active THPE): Yield=3.61 g, (76%), ¹H NMR (400 MHz, DMSO-d₆) δ ppm 8.46 (s, 2H), 7.77 (t, J=1.5 Hz, 2H), 7.46-7.34 (m, 4H), 7.26-7.11 (m, 6H), 2.24 (s, 2H). ¹³C NMR (101 MHz, DMSO-d₆) δ ppm 148.05, 146.88 (d, J=15.3 Hz), 137.80, 130.60, 129.56, 121.15, 117.96, 51.53, 30.17.

### Synthesis and Characterisation of NRTI Polymer-of-Prodrug—CDI Route

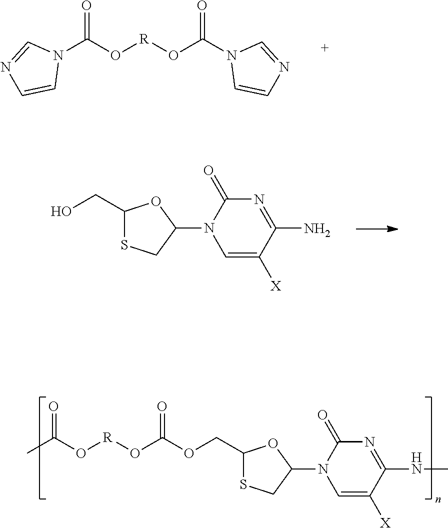 [0112] R=an aromatic or aliphatic hydrocarbon chain
X═F or H

### NRTI POP Synthesis with bis(imidazole carboxylic ester)

General synthesis of linear FTC POP structure using bis(imidazole carboxylic ester), poly[(1,6-hexanediol)/FTC]: To a dry 250 mL round-bottomed flask containing emtricitabine (FTC) (5 g, 20.2 mmol) and active 1,6-hexanediol (5.89 g, 19.2 mmol) was added potassium hydroxide (KOH) (0.2 g, 3.56 mmol) and anhydrous toluene (42.1 mL, 23 wt %) with stirring at 60° C. under a nitrogen atmosphere. Reaction progress was monitored by TLC and deemed complete after 18 hours. Toluene was removed in vacuo, the crude dissolved in dichloromethane (200 mL) and washed with deionised water (3×100 mL) and saturated sodium chloride solution (1×100 mL). The organic layer was dried over sodium sulphate, filtered and concentrated in vacuo to yield a crispy white solid. ¹H NMR (400 MHz, CDCl₃) δ ppm 1.39 (s, 4H), 1.67 (s, 4H), 3.12-3.18 (dd, J=12.0 Hz, 1H), 3.49-3.57 (dd, J=12.0 Hz, 1H), 4.08-4.19 (dt, J=6.51 Hz, 4H), 4.49-4.60 (m, 2H), 5.32-5.39 (m, 1H), 6.29 (s, 1H), 7.89 (d, J=6.35 Hz, 1H). ¹³C NMR (100 MHz, CDCl₃) δ ppm 25.5, 28.7, 38.7, 66.7, 68.7, 67.9, 83.9, 87.2, 87.8, 125.5, 135.3, 137.8, 153.9, 155.0, 155.5, 158.3.

poly[(FTC/tri(ethyleneglycol))]: ¹H NMR (400 MHz, DMSO-d₆) δ ppm 8.19 (d, J=7.3 Hz, 1H), 7.97-7.76 (m, 3H), 7.70-7.49 (m, 2H), 6.23-6.10 (m, 2H), 5.55-5.31 (m, 2H), 5.19 (t, J=3.9 Hz, 1H), 4.54-4.37 (m, 2H), 4.28-4.12 (m, 2H), 3.77 (qd, J=12.2, 3.8 Hz, 2H), 3.67-3.55 (m, 2H), 3.42 (ddd, J=11.8, 5.4, 1.9 Hz, 4H), 3.15 (ddd, J=17.9, 11.8, 5.0 Hz, 2H), 2.29 (d, J=0.8 Hz, 1H).

General synthesis of linear 3TC POP structure using bis(Imidazole carboxylic ester), poly[(1,6-hexanediol)/3TC]: To a dry 10 mL round-bottomed flask containing lamivudine (3TC) (0.48 g, 2.1 mmol) and active 1,6-hexanediol (0.61 g, 2.0 mmol) was added potassium hydroxide (KOH) (0.112 g, 2.0 mmol) and anhydrous toluene (4 mL, 24 wt %) with stirring at 60° C. under a nitrogen atmosphere. Reaction progress was monitored by TLC and deemed complete after 18 hours. Toluene was removed in vacuo, the crude dissolved in dichloromethane (100 mL) and washed with deionised water (3×50 mL) and saturated sodium chloride solution (1×50 mL). The organic layer was dried over sodium sulphate, filtered and concentrated in vacuo to yield a crispy white solid. 1H NMR (400 MHz, DMSO-d⁶) δ ppm 1.32 (s, 4H), 1.59 (s, 4H), 3.06-3.11 (dd, J=5.38 Hz, 1H), 3.39-3.45 (dd, J=5.38 Hz, 1H), 4.01-4.13 (dt, J=7.43 Hz, 2H), 4.35-4.40 (t, J=6.55 Hz, 4H), 5.35-5.38 (t, J=4.59 Hz, 1H), 5.75 (d, J=7.43 Hz, 1H), 6.22-6.26 (t, J=5.51 Hz, 1H), 7.28 (d, J=22.9 Hz, 1H), 7.69 (d, J=7.40 Hz, 1H). ¹³C NMR (100 MHz, DMSO-d⁶) δ ppm 24.7, 27.7, 35.7, 67.9, 80.9, 86.7, 94.3, 154.3, 154.5, 165.6.

TABLE 4
GPC data for FTC POP structures

| Experiment | | Monomer | | | GPC (DMF)^{a} | | | |
|---|---|---|---|---|---|---|---|---|
| no. | Monomer A | B | A:B | Solvent | Mₙ | M_{w} | Ð | DPₙ |
| 13 | active 1,6- | FTC | 1:1 | tetrahydrofuran | 470 | 810 | 1.727 | 1 |
| | hexanediol | | | | | | | |
| 14 | active 1,6- | FTC | 1:1 | toluene | 1620 | 1730 | 1.062 | 4 |
| | hexanediol | | | | | | | |
| 15 | active 1,6- | FTC | 1:1.05 | toluene | 3010 | 3570 | 1.180 | 7 |
| | hexanediol | | | | | | | |
| 16 | active 1,6- | FTC | 1.05:1 | toluene | 2550 | 3020 | 1.029 | 5 |
| | hexanediol | | | | | | | |
| 18 | active 1,6- | FTC | 1:1 | chloroform | 1650 | 1830 | 1.110 | 4 |
| | hexanediol | | | | | | | |
| 19 | active 1,6- | FTC | 1:1 | dimethylformamide | 3660 | 4100 | 1.120 | 9 |
| | hexanediol | | | | | | | |
| 20 | active 1,6- | FTC | 1:1 | pyridine | 1970 | 2060 | 1.047 | 4 |
| | hexanediol | | | | | | | |
| 21 | active tri- | FTC | 1:1 | toluene | 2160 | 3460 | 1.60 | 4 |
| | ethyleneglycol | | | | | | | |
| 22 | active 1,6- | FTC | 1:1 | toluene/chloroform | 2030 | 2160 | 1.060 | 4 |
| | hexanediol | | | | | | | |
| 23 | active 1,6- | FTC | 1:1 | dimethylsulfoxide | 2500 | 2260 | 1.150 | 5 |
| | hexanediol | | | | | | | |

^{a}DMF containing 0.01M LiBr at 60° C., 1 mL min⁻¹ flow rate.
Mₙ—number average molecular weight,
M_{w}—weight average molecular weight,
Ð—dispersity,
DPₙ—number average degree of polymerisation

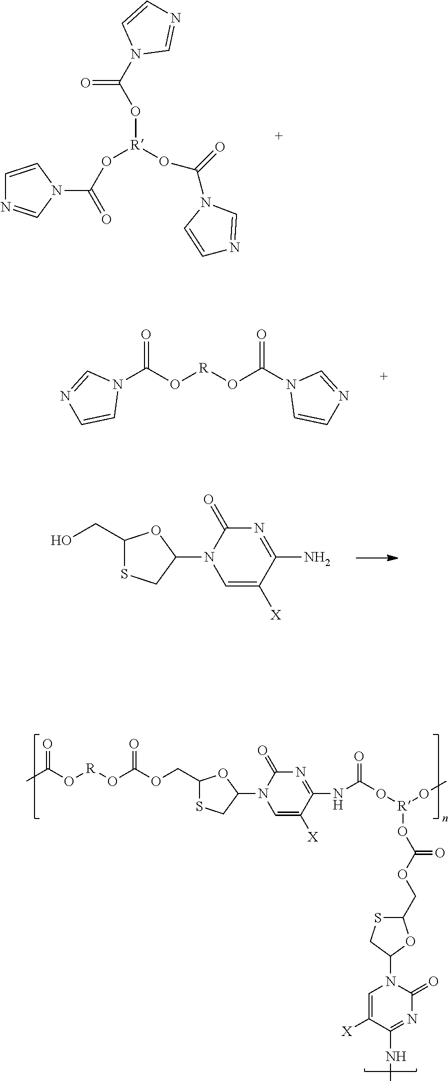

R and R′=an aromatic or aliphatic hydrocarbon chain
X═F or H

### Branched FTC POP Synthesis with bis- and tri(imidazole carboxylic ester)s

General synthesis of branched FTC POP structure using bis- and tri(imidazole carboxylic ester)s, poly[(1,6-hexanediol/(FTC/TMP)]: To a dry 250 mL round-bottomed flask containing emtricitabine (FTC) (5 g, 20.2 mmol), active 1,6-hexanediol (5.58 g, 18.2 mmol) and active TMP (0.56 g, 1.34 mmol) was added potassium hydroxide (KOH) (0.021 g, 0.37 mmol) and anhydrous toluene (43.1 mL, 23 wt %) with stirring at 60° C. under a nitrogen atmosphere. Reaction progress was monitored by TLC and deemed complete after 18 hours. Toluene was removed in vacuo, the crude dissolved in dichloromethane (200 mL) and washed with deionised water (3×100 mL) and saturated sodium chloride solution (1×100 mL). The organic layer was dried over sodium sulphate, filtered and concentrated in vacuo to yield a crispy white solid. ¹H NMR (400 MHz, DMSO-d⁶) δ ppm 0.83 (t, J=7.60 Hz, 3H), 1.40 (q, J=7.60 Hz, 2H), 3.11-3.18 (dd, J=5.35 Hz, 3H), 3.40-3.48 (dd, J=5.35 Hz, 3H), 3.70-3.81 (m, 1H), 4.09 (s, 5H), 4.46 (s, 5H), 5.18 (t, J=3.90 Hz, 1H), 5.36-5.43 (dt, J=3.90 Hz, 3H), 6.12-6.20 (dt, J=5.35 Hz, 3H) 7.63 (s, 3H), 7.90 (d, J=7.05 Hz, 5H), 8.19 ppm (d, J=7.2 Hz, 1H). ¹³C NMR (100 MHz, DMSO-d⁶) δ ppm 7.07, 21.9, 35.7, 36.7, 40.8, 54.9, 62.2, 67.1, 67.9, 81.2, 86.5, 86.8, 124.9, 134.9, 137.4, 152.9, 154.2, 157.6.

General synthesis of cross-linked FTC POP gel structure using a tri(imidazole carboxylic ester), poly[(FTC/TMP)]: To a dry 250 ml round-bottom flask containing emtricitabine (FTC) (5 g, 20.2 mmol) and active TMP (5.6 g, 13.5 mmol) was added potassium hydroxide (KOH) (0.2 g, 3.6 mmol) and anhydrous toluene (41 mL) with stirring at 60° C. under a nitrogen atmosphere. Reaction progress was monitored by TLC and deemed complete after 37 hours. Toluene was decanted and the crude was stirred in dichloromethane (100 ml) for 42 hours, replacing with fresh dichloromethane after 19 hours. The dichloromethane was removed and replaced with deionised water (100 mL). The deionised water was immediately neutralised with hydrochloric acid solution (1.5 mL, 1M). The mixture was stirred for 3 hours before replacing with fresh deionised water and stirring for a further 25 hours. Finally the water was decanted and the polymer gel was lyophilised for 48 hours.

### Synthesis and Characterisation of NRTI Polymer-of-Prodrug with Pendant Prodrugs

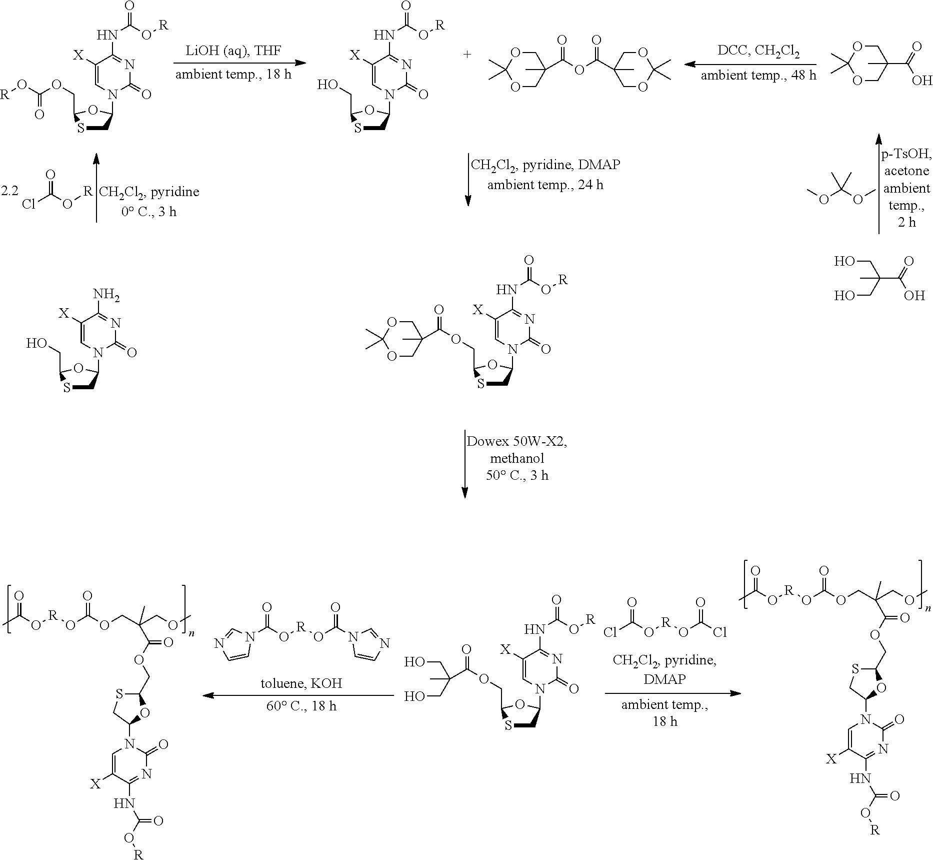 [0122] R and R′=an aromatic or aliphatic hydrocarbon chain
X═F or H

Overall Synthesis of Pendant-NRTI POP Structures

### Synthesis of 5′-alkoxycarbonyl FTC carbamates Using chlorformate

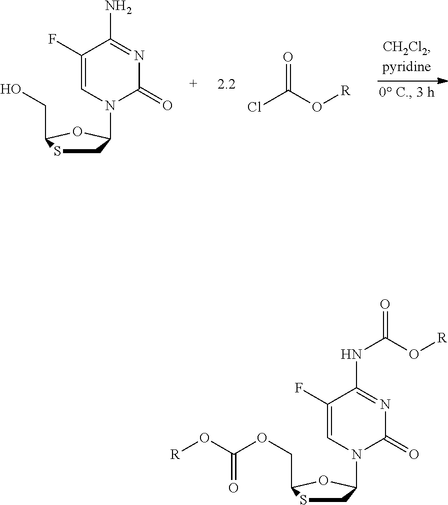 [0126] R=an aromatic or aliphatic hydrocarbon chain

### Synthesis of 5′-alkoxycarbonyl FTC carbamates Using chloroformates

General synthesis of 5′-alkoxycarbonyl FTC carbamate using chloroformate, Isobutyl (5-fluoro-1-((2S,5R)-2-(((Isobutoxycarbonyl)oxy)methyl)-1,3-oxathiolan-5-yl)-2-oxo-1,2-dihydropyrimidin-4-yl)carbamate: To a dry 25 mL round-bottomed flask containing emtricitabine (FTC) (1 g, 4.04 mmol) and pyridine (0.72 mL, 8.89 mmol) was added an anhydrous dichloromethane solution (10 mL) of isobutyl chloroformate (1.16 mL, 8.89 mmol) dropwise with stirring at 0° C. under a nitrogen atmosphere over 30 min. The reaction mixture was allowed to warm to ambient temperature with stirring and was deemed complete after 3 hours with monitoring by TLC. The crude reaction mixture was dissolved in dichloromethane (200 mL) and washed with deionised water (6×100 mL) and saturated sodium chloride solution (3×50 mL). The organic layer was dried over magnesium sulphate, filtered and concentrated in vacuo. The crude product was purified by silica flash chromatography, eluting with hexane gradually increasing to 50:50 ethyl acetate:hexane over 20 min, flow rate=36 mL/min. 1.51 g of colourless liquid (83%). 1H NMR (500 MHz, CDCl₃) δ ppm 0.95 (dd, J=6.67, 11.21 Hz, 12H), 2.00 (m, 2H), 3.23 (dd, J=3.69, 12.53 Hz, 1H), 3.55 (dd, J=5.41, 12.53 Hz, 1H), 3.98 (d, J=7.63 Hz, 4H), 4.57 (m, 2H), 5.39 (t, J=2.81 Hz, 1H), 6.30 (br. m, 1H), 8.10 (d, J=6.51 Hz, 1H). ¹³C-NMR (100 MHz, CDCl₃) δ ppm 18.77, 19.05, 27.69, 27.71, 38.55, 66.17, 72.68, 74.84, 84.57, 87.09, 139.64, 148.03, 153.32, 153.49, 154.88. Calcd: [M+H]⁺=448.4; found 448.2.

### Synthesis of 5′-alkoxycarbonyl FTC carbamates Using imidazole carboxylic ester

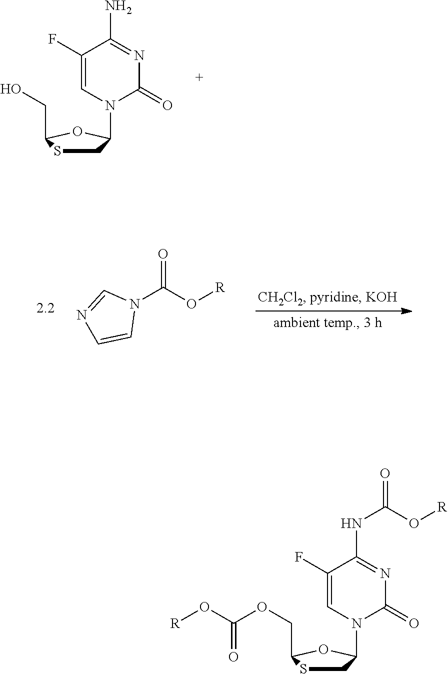 [0129] R=an aromatic or aliphatic hydrocarbon chain

### Synthesis of 5′-alkoxycarbonyl FTC carbamates Using imidazole carboxylic esters

General synthesis of 5′-alkoxycarbonyl FTC carbamate using Imidazole carboxylic ester, dodecyl (1-((2S,5R)-2-((((dodecyloxy)carbonyl)oxy)methyl)-1,3-oxathiolan-5-yl)-5-fluoro-2-oxo-1,2-dihydropyrimidin-4-yl)carbamate: To a dry 25 mL round-bottomed flask containing emtricitabine (FTC) (1 g, 4.04 mmol), dodecyl 1H-imidazole-1-carboxylate (2.49 g, 8.89 mmol) and anhydrous dichloromethane (25 mL) was added pyridine (5 mL), then potassium hydroxide (KOH) (ca. 0.2 g) after 10 min with stirring at ambient temperature under a nitrogen atmosphere. Reaction progress was monitored by TLC and was deemed complete after 18 hours. The crude reaction mixture was dissolved in dichloromethane (250 mL) and washed with deionised water (5×100 mL) and saturated sodium chloride solution (2×100 mL). The organic layer was dried over magnesium sulphate, filtered and concentrated in vacuo, yielding 0.98 g of white solid (67%). ¹H NMR (500 MHz, CDCl₃) δ ppm 0.88 (t, J=6.65 Hz, 6H), 1.26 (m, 36H), 1.68 (m, 4H), 3.21 (br. s, 1H), 3.55 (br. s, 1H), 4.18 (t, J=6.65 Hz, 4H), 4.56 (br m, 2H), 5.39 (t, J=3.44 Hz, 1H), 6.30 (br s, 1H). ¹³C NMR (100 MHz, CDCl₃) δ ppm 14.10, 22.67, 25.55, 25.82, 28.49, 28.58, 29.15, 29.25, 29.32, 29.47, 29.50, 29.55, 29.61, 31.90, 66.30, 69.03, 84.12, 86.80, 146.07, 153.26, 154.83. Calcd: [M+H]⁺=672.4; found 672.4.

### Synthesis of FTC carbamate from 5′-alkoxycarbonyl FTC carbamates

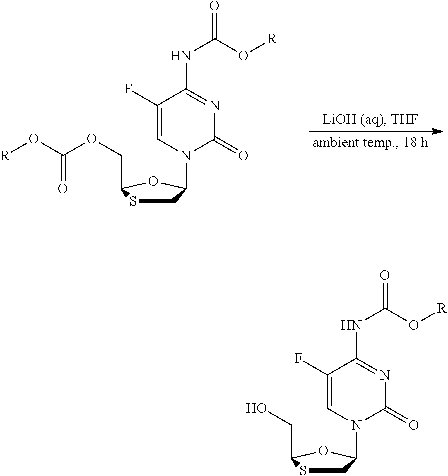 [0132] R=an aromatic or aliphatic hydrocarbon chain

### Synthesis of FTC carbamate from 5′-alkoxycarbonyl FTC carbamate

General synthesis of FTC carbamate from 5′-alkoxycarbonyl FTC carbamates, Isobutyl (5-fluoro-1-((2S,5R)-2-(hydroxymethyl)-1,3-oxathiolan-5-yl)-2-oxo-1,2-dihydropyrimidin-4-yl) carbamate: Isobutyl (5-fluoro-1-((2S,5R)-2-(((isobutoxycarbonyl)oxy)methyl)-1,3-oxathiolan-5-yl)-2-oxo-1,2-dihydropyrimidin-4-yl)carbamate (1.81 g, 4.04 mmol), lithium hydroxide (LiOH) (0.48 g, 20.2 mmol), tetrahydrofuran (THF) (10 mL) and deionised water (3 mL) were stirred in a 25 mL round-bottomed flask at ambient temperature. Reaction progress was monitored by TLC and was deemed complete after 18 hours. Volatiles were removed in vacuo and the crude product purified by silica flash chromatography, eluting with dichloromethane gradually increasing to 5:95 methanol:dichloromethane over 20 min, flow rate=36 mL/min. 0.58 g of pale yellow solid (47%). 1H NMR (500 MHz, CDCl₃) δ ppm 0.96 (d, J=6.86 Hz, 6H), 2.00 (m, 1H), 3.26 (dd, J=3.23, 12.60 Hz, 1H), 3.52 (dd, J=5.01, 12.74 Hz, 1H), 3.97 (m, 3H), 4.19 (dd, J=1.93, 12.76 Hz, 1H), 5.31 (t, J=3.09 Hz, 1H), 6.2 (m, 1H), 8.4 (br. s, 1H). ¹³C NMR (100 MHz, CDCl₃) δ ppm 19.06, 27.54, 27.72, 38.79, 62.31, 72.68, 87.29, 88.45, 153.49, 153.67. Calcd: [M+Na]=370.3; found 370.1.

### Synthesis of FTC carbamate from FTC Using chloroformates

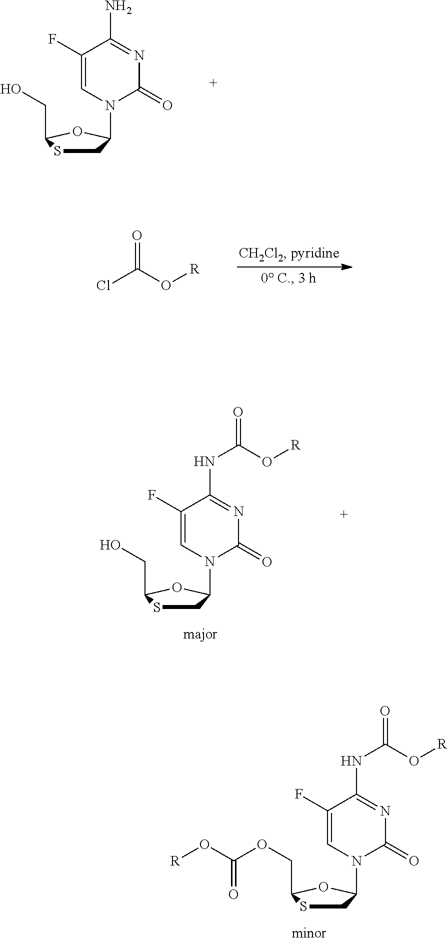 [0135] R=an aromatic or aliphatic hydrocarbon chain

### Synthesis of FTC carbamate from FTC Using chloroformates

General procedure for the formation of free 5′-hydroxyl carbamate-only prodrug. Emtricitabine (FTC) (1 eq., 12.3 mmol) was dispersed in dichloromethane (50 mL) under a nitrogen atmosphere. Alkyl chloroformate (1 eq., 12.3 mmol) was added to the stirring FTC solution, and the mixture cooled to 0° C. Pyridine (1 eq., 12.3 mmol, 0.99 mL) was added dropwise to the reaction over 30 mins resulting in a clear pale yellow solution with precipitated pyridinium hydrochloride. The solution was stirred at 0° C. for 1 h, and then at room temperature. The reaction was monitored by TLC and was deemed complete after 2 hours. Following the removal of volatiles in vacuo, the residue was purified by liquid chromatography on silica using either 100% ethyl acetate, or 0-8% methanol in dichloromethane as the eluent system.

Butyl (5-fluoro-1-((2S,5R)-2-(hydroxymethyl)-1,3-oxathiolan-5-yl)-2-oxo-1,2-dihydropyrimidin-4-yl) carbamate. Yield: 2.53 g of white semi-solid (60%). ¹H NMR (500 MHz, CDCl₃): δ ppm 8.57 (d, J=12.8 Hz, 1H), 6.23 (t, J=4.0 Hz, 1H), 5.29 (d, J=3.0 Hz, 1H), 4.17 (q, J=8.9, 6.9 Hz, 3H), 3.98 (dd, J=12.8, 3.0 Hz, 1H), 3.51 (dd, J=12.6, 5.3 Hz, 1H), 3.23 (dd, J=12.6, 2.9 Hz, 1H), 1.64 (q, J=7.1 Hz, 2H), 1.39 (h, J=7.4 Hz, 2H), 1.25 (d, J=7.5 Hz, 2H), 0.92 (t, J=7.4 Hz, 3H). ¹³C NMR (126 MHz, CDCl₃): δ=153.48, 88.58, 87.26, 65.97, 62.58, 38.87, 31.89, 30.57, 29.66, 22.66, 18.97, 13.66. Calcd: [M+Na]⁺=370.4; found 370.1.

Isobutyl (5-fluoro-1-((2S,5R)-2-(hydroxymethyl)-1,3-oxathiolan-5-yl)-2-oxo-1,2-dihydropyrimidin-4-yl) carbamate. Yield: 1.85 g of yellow solid (44%). ¹H NMR (400 MHz, CDCl₃): δ ppm 8.66-8.46 (m, 1H), 6.20 (s, 1H), 4.16 (dd, J=12.8, 2.6 Hz, 1H), 4.03-3.86 (m, 3H), 3.48 (dd, J=12.7, 5.2 Hz, 1H), 3.21 (dd, J=12.6, 2.9 Hz, 1H), 2.03-1.89 (m, 1H), 0.91 (d, J=6.7 Hz, 6H). ¹³C NMR (101 MHz, CDCl₃): δ=153.54, 98.15, 88.73, 87.33, 72.61, 65.15, 62.08, 38.86, 36.90, 27.70, 26.98, 23.75, 20.48, 19.03, 7.01. Calcd: [M+Na]+=370.3; found 370.1.

Octyl (5-fluoro-1-((2S,5R)-2-(hydroxymethyl)-1,3-oxathiolan-5-yl)-2-oxo-1,2-dihydropyrimidin-4-yl) carbamate. Yield: 2.30 g of white semi-solid (46%). ¹H NMR (400 MHz, CDCl₃): δ ppm 8.50 (s, 1H), 6.10 (s, 1H), 4.16-3.96 (m, 3H), 3.88 (dd, J=12.9, 3.1 Hz, 1H), 3.51 (dd, J=27.9, 12.2 Hz, 1H), 3.39 (dd, J=12.7, 5.3 Hz, 1H), 3.13 (dd, J=12.7, 2.8 Hz, 1H), 1.53 (p, J=6.9 Hz, 2H), 1.26-1.06 (m, 11H), 0.73 (t, J=6.8 Hz, 3H). ¹³C NMR (101 MHz, CDCl₃): δ=153.43, 153.27, 97.83, 88.68, 87.18, 66.35, 64.79, 62.17, 61.70, 38.58, 36.64, 31.46, 28.92, 28.87, 28.32, 26.39, 25.48, 23.39, 22.32, 20.52, 13.79, 6.75.

Decyl (5-fluoro-1-((2S,5R)-2-(hydroxymethyl)-1,3-oxathiolan-5-yl)-2-oxo-1,2-dihydropyrimidin-4-yl) carbamate. Yield: 0.64 g of viscous liquid (37%). 1H NMR (500 MHz, CDCl₃) δ ppm 0.90 (t, J=7.22 Hz, 3H), 1.28 (br. m, 14H), 1.68 (m, 2H), 3.24 (br. s, 1H), 3.53 (br. s, 1H), 4.00 (br. d, J=12.62 Hz, 1H), 4.19 (br. s, 3H), 5.34 (br. m, 1H), 6.30 (br. m, 1H), 8.48 (br. s, 1H).

### Synthesis of FTC carbamate from FTC Using imidazole carboxylic ester

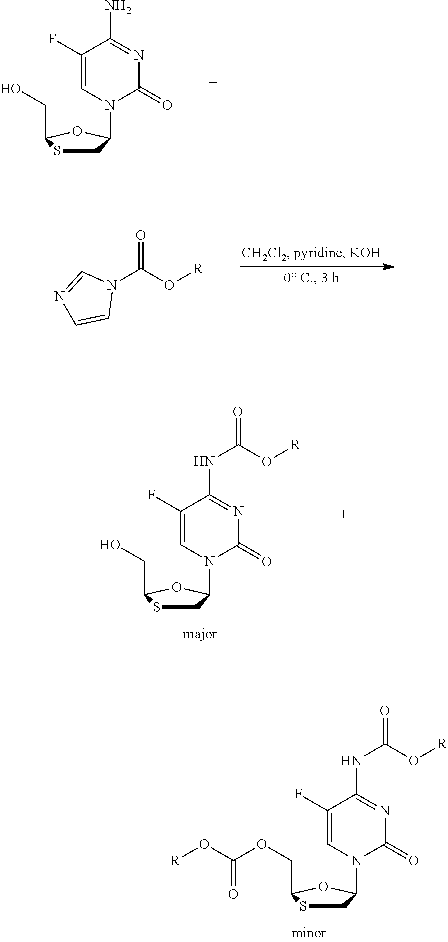 [0142] R=an aromatic or aliphatic hydrocarbon chain

### Synthesis of FTC carbamate from FTC Using imidazole carboxylic ester

General synthesis of FTC carbamate from FTC using imidazole carboxylic ester: To a dry 25 mL round-bottomed flask containing emtricitabine (FTC) (1 g, 4.04 mmol), imidazole carboxylic ester (8.89 mmol) and anhydrous dichloromethane (25 mL) was added pyridine (5 mL), then potassium hydroxide (KOH) (ca. 0.2 g) after 10 min with stirring at ambient temperature under a nitrogen atmosphere. Reaction progress was monitored by TLC and was deemed complete after 18 hours. The crude reaction mixture was dissolved in dichloromethane (250 mL) and washed with hydrochloric acid (HCl) (1M, 2×100 mL), deionised water (3×100 mL) and saturated sodium chloride solution (2×100 mL). The organic layer was dried over magnesium sulphate, filtered and concentrated in vacuo. The crude product was purified by silica flash chromatography, eluting with dichloromethane gradually increasing to 10:90 methanol:dichloromethane over 20 min, flow rate=36 mL/min.

### Synthesis of diol Monomer with Pendant FTC

### Synthesis of Bis-MPA Acetonide

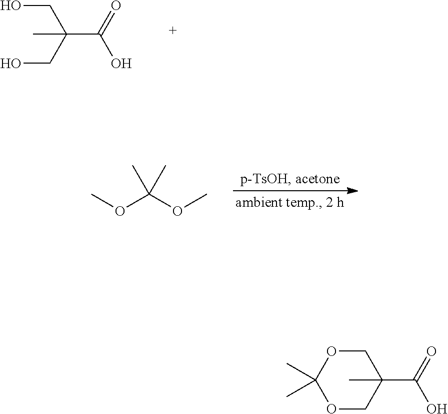

### Synthesis of Bis-MPA acetonide

Synthesis of isopropylidene-2,2-bis(methoxy)propionic acid: 2,2-Bis(hydroxymethyl)-propionic acid (bis-MPA) (100 g, 0.746 mol), 2,2-dimethoxypropane (137 mL, 1.12 mol), and p-toluenesulfonic acid monohydrate (7.09 g, 37.0 mmol) were stirred in acetone (500 mL) for 2 hours at ambient temperature (clear, colourless). After this time, the catalyst was neutralised by addition of triethylamine (TEA) (4 mL, 37.3 mmol), resulting in salt precipitation. The product was obtained by removal of volatiles under reduced pressure, dissolving the crude in dichloromethane (750 mL), washing with deionised water (2×300 mL), drying over magnesium sulphate, filtering and concentrating in vacuo. 100.86 g, white solid, (78%). ¹H NMR (500 MHz, CDCl₃) δ ppm 1.22 (s, 3H), 1.41 (s, 3H), 1.45 (s, 3H), 3.63 (d, J=11.97 Hz, 2H), 4.18 (d, 11.97 Hz, 2H). ¹³C NMR (100 MHz, CDCl₃) δ ppm 18.43, 22.00, 25.16, 41.74, 65.85, 98.33, 180.22. This compound was prepared by the procedure reported by Ihre et al. Spectroscopic data agreed with those reported.¹⁰³

### Synthesis of Bis-MPA acetonide anhydride

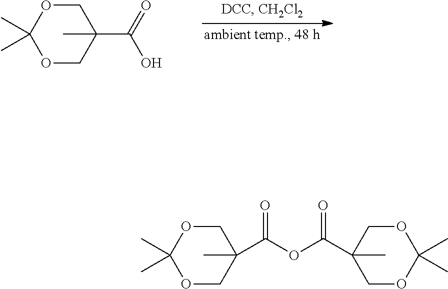

### Synthesis of Bis-MPA acetonide anyhydride

Synthesis of isopropylidene-2,2-bis(methoxy)propionic anhydride: isopropylidene-2,2-bis(methoxy)propionic acid (88.94 g, 0.511 mol) and N,N′-dicyclohexylcarbodiimide (DCC) (52.68 g, 0.255 mol) were stirred in dichloromethane (500 mL) at ambient temperature for 48 hours. The precipitated N,N′-dicyclohexylurea (DCU) byproduct was removed by filtration and washed with a small volume of dichloromethane. The crude product was purified by precipitating the filtrate into cold hexane (2.5 L, cooled with dry ice bath) with vigorous stirring. 84.18 g, white viscous oil, (99%). ¹H NMR (400 MHz, CDCl₃) δ ppm 1.24 (s, 3H), 1.40 (s, 3H), 1.44 (s, 3H), 3.69 (d, J=12.1 Hz, 4H), 4.21 (d, J=12.1 Hz, 4H). ¹³C NMR (100 MHz, CDCl₃) δ ppm 17.70, 21.73, 25.56, 43.59, 65.64, 98.42, 169.57. This compound was prepared according to the procedure reported by Malkoch et al. Spectroscopic data agreed with those reported.¹⁰⁴

### Synthesis of FTC carbamate bis-MPA acetonide ester

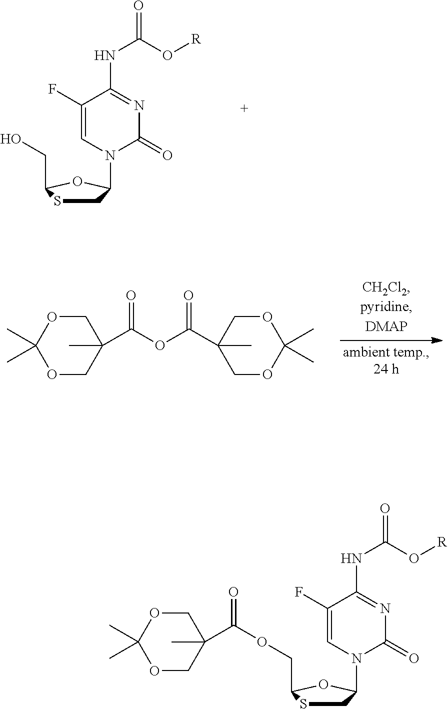 [0149] R=an aromatic or aliphatic hydrocarbon chain

### Synthesis of FTC carbamate bis-MPA acetonide ester

General procedure for the esterification of FTC carbamate-only prodrug with bis-MPA acetonide anhydride to yield diol monomer precursor. FTC carbamate-only prodrug (1 eq., 5.76 mmol), 4-dimethylaminopyridine (DMAP) (0.2 eq., 1.15 mmol), pyridine (5 eq., 28.8 mmol) and bis-MPA acetonide anhydride (1.3 eq., 7.49 mmol) were dissolved in dichloromethane (25 mL) under a nitrogen atmosphere. The reaction was stirred at ambient temperature, monitored by TLC and was deemed complete after 24 hours. The residue was diluted with DCM (200 mL), washed with NaHSO₄ (3×1M 100 mL), NaHCO₃ (3×1M 100 mL), brine (2×100 mL) and then dried over MgSO₄. After removal of volatiles, the residue was purified using liquid chromatography on silica with 60% EtOAc: 40% Hexane eluent.

((2S,5R)-5-(4-((butoxycarbonyl)amino)-5-fluoro-2-oxopyrimidin-1(2H)-yl)-1,3-oxathiolan-2-yl) methyl 2,2,5-trimethyl-1,3-dioxane-5-carboxylate. Yield: 2.44 g of yellow viscous liquid (87%). 1H NMR (400 MHz, CDCl₃): δ ppm 6.31-6.23 (m, 1H), 5.40 (dd, J=5.0, 3.0 Hz, 1H), 4.70 (dd, J=12.5, 4.9 Hz, 1H), 4.47 (dd, J=12.5, 3.0 Hz, 1H), 4.25-4.16 (m, 4H), 3.68 (dd, J=11.8, 2.5 Hz, 2H), 3.61-3.50 (m, 1H), 3.19 (dd, J=12.4, 4.0 Hz, 1H), 1.69 (dq, J=8.7, 6.8 Hz, 2H), 1.40 (d, J=26.9 Hz, 8H), 1.17 (s, 3H), 0.94 (s, 3H). ¹³C NMR (101 MHz, CDCl₃): δ=173.89, 171.14, 153.38, 153.20, 98.25, 86.64, 83.82, 66.43, 66.05, 65.99, 63.91, 60.37, 53.46, 42.27, 37.76, 30.55, 25.88, 21.23, 21.02, 18.99, 18.37, 14.18, 13.66. Calcd: [M+Na]⁺=526.5; found 526.2.

((2S,5R)-5-(5-fluoro-4-((isobutoxycarbonyl)amino)-2-oxopyrimidin-1(2H)-yl)-1,3-oxathiolan-2-yl) methyl 2,2,5-trimethyl-1,3-dioxane-5-carboxylate. Yield: 2.59 g of colourless viscous liquid (97%). ¹H NMR (500 MHz, CDCl₃): δ ppm 6.30-6.21 (m, 1H), 5.39 (dd, J=5.0, 3.0 Hz, 1H), 4.68 (dd, J=12.3, 5.0 Hz, 1H), 4.45 (dd, J=12.5, 3.0 Hz, 1H), 4.23-4.17 (m, 2H), 3.95 (d, J=6.7 Hz, 2H), 3.66 (dd, J=11.8, 3.3 Hz, 2H), 3.54 (s, 1H), 3.17 (dd, J=12.4, 4.1 Hz, 1H), 2.03-1.96 (m, 1H), 1.41 (s, 3H), 1.34 (s, 3H), 1.15 (s, 3H), 0.95 (d, J=6.8 Hz, 6H). ¹³C NMR (126 MHz, CDCl₃): δ=173.88, 153.24, 98.24, 72.69, 65.97, 63.92, 53.47, 42.26, 38.36, 27.68, 25.84, 19.04, 18.37. Calcd: [M+Na]+=526.5; found 526.2.

((2S,5R)-5-(5-fluoro-4-(((octyloxy)carbonyl)amino)-2-oxopyrimidin-1(2H)-yl)-1,3-oxathiolan-2-yl) methyl 2,2,5-trimethyl-1,3-dioxane-5-carboxylate. Yield: 2.57 g of yellow viscous liquid (88%). ¹H NMR (400 MHz, CDCl₃): δ ppm 6.27 (t, J=4.8 Hz, 1H), 5.41 (dd, e=4.9, 3.0 Hz, 1H), 4.70 (dd, J=12.6, 4.9 Hz, 1H), 4.47 (dd, J=12.5, 3.0 Hz, 1H), 4.26-4.14 (m, 4H), 3.68 (dd, J=11.9, 2.5 Hz, 2H), 3.55 (s, 1H), 3.19 (dd, J=12.4, 4.1 Hz, 1H), 1.75-1.65 (m, 2H), 1.43 (s, 3H), 1.37 (s, 5H), 1.34-1.21 (m, 8H), 1.17 (s, 3H), 0.88 (t, J=6.8 Hz, 3H). ¹³C NMR (101 MHz, CDCl₃): δ=173.88, 153.36, 153.19, 98.24, 85.97, 83.78, 66.74, 65.98, 63.92, 42.27, 37.73, 31.74, 29.18, 29.13, 28.56, 25.79, 22.60, 21.23, 18.36, 14.18, 14.06. Calcd: [M+Na]+=582.7; found 582.2.

((2S,5R)-5-(4-(((decyloxy)carbonyl)amino)-5-fluoro-2-oxopyrimidin-1(2H)-yl)-1,3-oxathiolan-2-yl) methyl 2,2,5-trimethyl-1,3-dioxane-5-carboxylate. Yield: 0.611 g of pale yellow solid (62%). ¹H NMR (500 MHz, CDCl₃) δ ppm 0.88 (t, J=7.41 Hz, 3H), 1.18 (s, 3H), 1.27 (br. s, 13H), 1.38 (s, 4H), 1.44 (s, 3H), 1.70 (dt, J=7.44, 14.88, 2H), 3.18 (br d, J=9.09 Hz, 1H), 3.55 (s, 1H), 3.70 (dd, J=2.48, 11.98 Hz, 2H), 4.21 (m, 4H), 4.48 (dd, J=2.67, 12.38 Hz, 1H), 4.70 (br. d, J=8.96 Hz, 1H), 5.40 (br. m, 1H), 6.28 (br. m, 1H), 7.84 (br s 1H). ¹³C NMR (100 MHz, CDCl₃) δ ppm 14.09, 18.38, 21.23, 22.66, 25.80, 28.57, 29.23, 29.27, 29.49, 29.50, 31.86, 37.72, 42.29, 53.43, 63.92, 66.00, 66.74, 83.74, 86.60, 98.26, 124.14, 146.06, 153.18, 153.37, 173.89. Calcd: [M+Na]⁺=610.3; found 610.3.

### Synthesis of FTC carbamate bis-MPA ester diol monomer

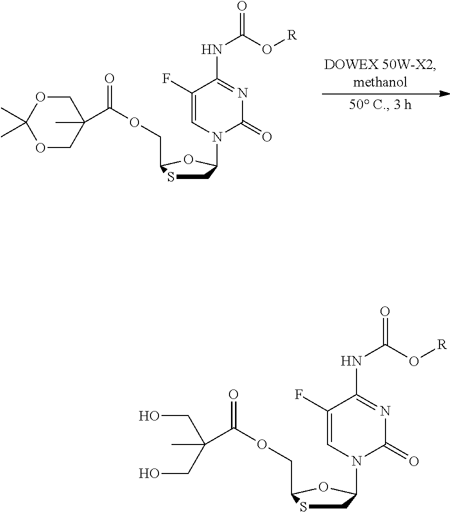 [0156] R=an aromatic or aliphatic hydrocarbon chain

### Synthesis of FTC carbamate bis-MPA ester diol monomer

General synthesis of FTC carbamate bis-MPA ester diol monomer. FTC monomer precursor (5.76 mmol) and DOWEX 50W-X2 (10 wt %) was added to methanol (100 mL). The resultant mixture was heated to 50° C. and stirred for 3 hours. Loss of the acetonide protecting group was monitored by TLC. Once complete, the resin was filtered off and the solvent removed.

((2S,5R)-5-(4-((butoxycarbonyl)amino)-5-fluoro-2-oxopyrimidin-1(2H)-yl)-1,3-oxathiolan-2-yl) methyl 3-hydroxy-2-(hydroxymethyl)-2-methylpropanoate (butyl FTC). Yield: 1.97 g (87.0%)¹H NMR (400 MHz, CDCl₃): δ ppm 8.06-7.78 (m, 1H), 6.25 (s, 1H), 5.41 (s, 1H), 4.65-4.52 (m, 2H), 4.18 (t, J=6.8 Hz, 2H), 3.92 (d, J=11.4 Hz, 2H), 3.83-3.71 (m, 2H), 3.56 (s, 1H), 3.47 (t, J=2.1 Hz, 2H), 3.20 (dd, J=12.3, 4.3 Hz, 3H), 1.69 (t, J=7.3 Hz, 2H), 1.44-1.20 (m, 10H), 1.13 (s, 3H), 0.94-0.82 (m, 3H). ¹³C NMR (101 MfHz, CDCl₃): δ ppm 175.28, 153.44, 153.27, 87.33, 83.46, 67.43, 66.84, 63.85, 49.62, 31.75, 29.19, 29.15, 28.56, 25.78, 22.61, 17.16, 14.07.

((2S,5R)-5-(((1E,3E)-2-fluoro-3-(formylamino)-3-((Isobutoxycarbonyl)amino)prop-1-en-1-yl)amino) 1,3-oxathiolan-2-yl)methyl 3-hydroxy-2-(hydroxymethyl)-2-methylpropanoate (isobutyl FTC). Yield: 3.55 g (76.0%) ¹H-NMR (400 MHz, CDCl₃): δ ppm 7.98 (s, 1H), 6.25 (td, J=4.1, 2.1 Hz, 1H), 5.42 (dd, J=4.6, 3.0 Hz, 1H), 4.62-4.50 (m, 2H), 3.97 (d, J=6.7 Hz, 2H), 3.90 (d, J=11.3 Hz, 2H), 3.75 (dd, J=11.3, 3.6 Hz, 2H), 3.58 (dd, J=12.3, 5.4 Hz, 1H), 3.21 (dd, J=12.4, 4.1 Hz, 1H), 2.04-1.95 (m, 1H), 1.14 (s, 3H), 0.97 (d, J=6.7 Hz, 6H). ¹³C NMR (101 MHz, CDCl₃): δ ppm 175.24, 153.52, 153.36, 87.69, 87.18, 86.40, 83.93, 72.72, 71.28, 66.99, 66.81, 66.75, 64.07, 63.82, 55.29, 50.56, 49.68, 49.65, 37.78, 27.89, 27.68, 19.01, 18.97, 17.21, 17.18, 17.15.

((2S,5R)-5-(5-fluoro-4-(((octyloxy)carbonyl)amino)-2-oxopyrimidin-1(2H)-yl)-1,3-oxathiolan-2-yl) methyl 3-hydroxy-2-(hydroxymethyl)-2-methylpropanoate (octyl FTC). Yield: 2.08 g (87.4%) ¹H-NMR (400 MHz, CDCl₃): δ ppm 6.24 (td, J=4.5, 2.4 Hz, 1H), 5.42 (dd, J=4.6, 3.0 Hz, 1H), 4.57 (qd, J=12.6, 3.9 Hz, 2H), 4.19 (t, J=6.7 Hz, 2H), 3.89 (d, J=11.2 Hz, 2H), 3.75 (dd, J=11.2, 3.2 Hz, 2H), 3.62-3.53 (m, 1H), 3.22 (dd, J=12.4, 4.1 Hz, 1H), 1.73-1.62 (m, 2H), 1.48-1.34 (m, 2H), 1.14 (s, 3H), 0.94 (t, J=7.4 Hz, 3H). ¹³C NMR (101 MHz, CDCl₃) δ 175.28, 153.44, 153.27, 87.54, 83.33, 67.43, 66.84, 63.85, 50.71, 49.62, 37.16, 31.75, 29.19, 29.15, 28.56, 25.78, 22.61, 17.16, 14.07.

((2S,5R)-5-(4-(((decyloxy)carbonyl) amino)-5-fluoro-2-oxopyrimidin-1(2H)-yl)-1,3-oxathiolan-2-yl) methyl 3-hydroxy-2-(hydroxymethyl)-2-methylpropanoate (decyl FTC). Yield: 0.49 g of orange waxy-solid (86%). ¹H NMR (500 MHz, MeO-D₄) δ ppm 0.91 (t, J=7.11 Hz, 3H), 1.22 (s, 3H), (1.31, br. m, 14H), 1.68 (m, 2H), 3.32 (m, 2H), 3.68 (m, 2H), 3.73 (m, 2H), 4.00 (t, J=6.55 Hz, 1H), 4.22 (t, J=6.22 Hz, 1H), 4.54 (m, 1H), 4.61 (m, 1H), 5.51 (m, 1H), 6.25 (m, 1H), 8.14 (m, 1H).

### Synthesis of Pendant-FTC POP

Bis(chloroformate) Route

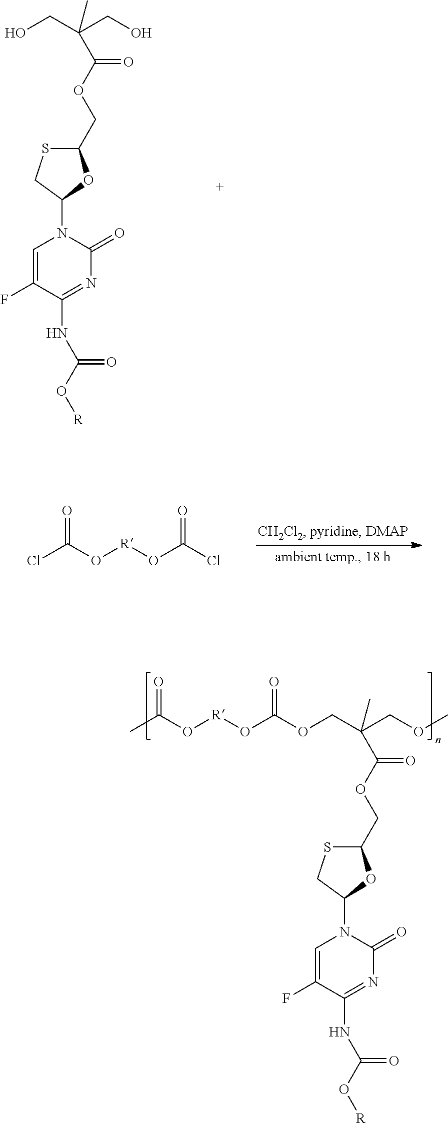

R and R′=an aromatic or aliphatic hydrocarbon chain

### Synthesis of Pendant-FTC POP with bis(chloroformate)

General synthesis of pendant-FTC POP with bis(chloroformate). To a dry 10 mL round-bottom flask, FTC carbamate bis-MPA ester diol monomer (0.44 g, 7.98 mmol) was melted at 60° C. under nitrogen. Tri(ethylene glycol) bischloroformate (0.16 mL, 7.98 mmol) was added, followed by drop-wise addition of pyridine (1.4 mL, 17.6 mmol) to generate a melt. After heating at 60° C. for 16 hrs, the crude reaction mixture was cooled to ambient temperature, followed by dilution with DCM (200 mL). The organics were washed with HCl solution (1M, 2×100 mL) then saturated sodium chloride solution (3×100 mL), dried over MgSO₄ and volatiles removed in vacuo to yield the prodrug polymer.

Poly[(isobutyl FTC/tri(ethylene glycol) carbonate]: Yellow brittle solid. ¹H NMR (400 MHz, CDCl₃) δ ppm 8.01-7.72 (m, 1H), 6.26 (t, J=5.2 Hz, 1H), 5.46-5.33 (m, 1H), 4.69-4.43 (m, 2H), 4.37-4.21 (m, 6H), 3.97 (d, J=6.7 Hz, 1H), 3.67 (d, J=24.6 Hz, 10H), 3.62-3.48 (m, 1H), 3.36-3.08 (m, 1H), 2.01 (d, J=6.9 Hz, 1H), 1.42-1.20 (m, 3H), 0.95 (dd, J=16.8, 6.7 Hz, 4H). ¹³C NMR (101 MHz, CDCl₃) δ ppm 173.54, 171.89, 155.17, 154.66, 153.44, 153.27, 87.05, 84.13, 72.70, 71.40, 71.35, 70.64, 70.61, 70.52, 68.86, 68.82, 68.36, 67.31, 48.60, 46.67, 42.76, 37.65, 27.70, 19.06, 18.99, 18.86, 17.46.]

Poly[butyl FTC/tri(ethylene glycol) carbonate]: Viscous orange liquid. ¹H NMR (400 MHz, CDCl₃) δ ppm 6.26 (s, 1H), 5.38 (s, 1H), 4.76-4.12 (m, 9H), 3.90-3.48 (m, 14H), 3.30-3.10 (m, 1H), 1.75-1.52 (m, 2H), 1.49-1.17 (m, 5H), 0.98-0.83 (m, 2H). ¹³C NMR (101 MHz, CDCl₃) δ ppm 154.68, 71.42, 71.38, 70.66, 70.56, 68.88, 67.32, 49.57, 46.68, 42.76, 30.58, 19.01, 17.35, 13.68.

Poly[octyl FTC/tri(ethylene glycol) carbonate]: Slow-flowing orange liquid. ¹H NMR (400 MHz, CDCl₃) δ ppm 6.25 (q, J=8.5, 6.7 Hz, 1H), 5.39 (ddd, J=10.9, 5.2, 2.3 Hz, 1H), 4.81-4.12 (n, 10H), 3.90-3.46 (m, 13H), 3.33-3.13 (m, 15H), 1.75-1.52 (m, 2H), 1.47-1.20 (m, 13H), 1.00-0.78 (m, 3H). ¹³C NMR (101 MHz, CDCl₃) δ ppm 175.27, 173.53, 171.59, 155.28, 154.66, 152.10, 71.41, 71.39, 71.36, 70.64, 70.62, 70.55, 68.87, 68.29, 67.31, 66.77, 49.59, 48.61, 46.67, 42.76, 32.79, 31.80, 31.76, 31.74, 29.22, 29.19, 29.15, 29.12, 28.91, 28.63, 28.57, 25.79, 22.61, 17.33, 14.08.

TABLE 5
GPC data for FTC pendant POP structures

| Experiment | | Monomer | | | GPC (DMF)^{a} | | | |
|---|---|---|---|---|---|---|---|---|
| no. | Monomer A | B | A:B | Solvent | Mₙ | M_{w} | Ð | DPₙ |
| 24 | tri(ethylene | Isobutyl | 1:1 | Pyridine | 3690 | 6950 | 1.90 | 6 |
| | glycol) | FTC | | | | | | |
| | bis(chloroformate) | prodrug | | | | | | |
| 25 | tri(ethylene | Butyl FTC | 1:1 | Pyridine | 2750 | 4080 | 1.48 | 4 |
| | glycol) | prodrug | | | | | | |
| | bis(chloroformate) | | | | | | | |
| 26 | tri(ethylene | Octyl FTC | 1:1 | Pyridine | 2650 | 3980 | 1.50 | 4 |
| | glycol) | prodrug | | | | | | |
| | bis(chloroformate) | | | | | | | |

^{a}DMF containing 0.01M LiBr at 60° C., 1 mL min⁻¹ flow rate.
Mₙ—number average molecular weight,
M_{w}—weight average molecular weight,
Ð—dispersity,
DPₙ—number average degree of polymerisation

### CDI-Activated Diol Route

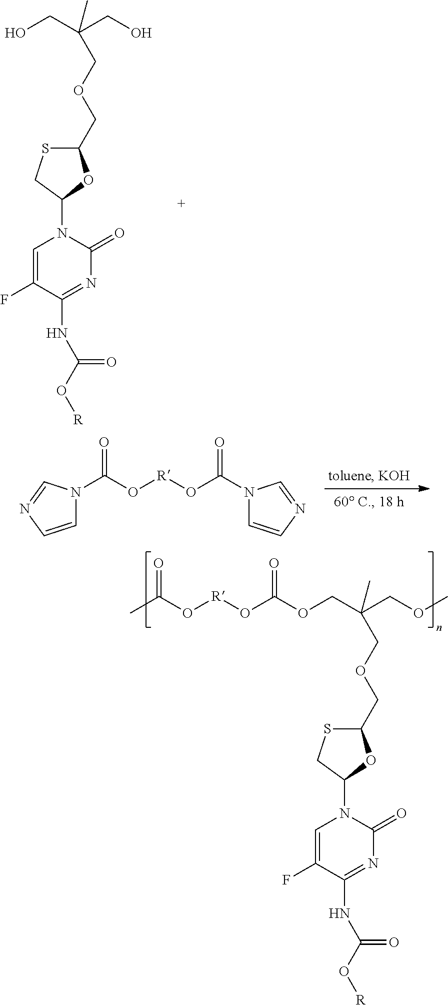 [0169] R and R′=an aromatic or aliphatic hydrocarbon chain

### Synthesis of Pendant-FTC POP with CDI-Activated Diol

General synthesis of pendant-FTC POP with CDI-activated diols: To a dry 10 mL round-bottomed flask containing FTC carbamate bis-MPA ester diol monomer (0.57 g, 1.04 mmol), alkyl bis carboxylic imidazolide esters (1.04 mmol) and KOH (ca. 1 g) was added anhydrous toluene (50 wt %) with stirring at 60° C. under a nitrogen atmosphere. Reaction progress was monitored by TLC and deemed complete after 18 hours. Toluene was removed in vacuo, the crude dissolved in dichloromethane (200 mL) and washed with water (6×100 mL) and saturated sodium chloride solution (2×100 mL). The organic layer was dried over magnesium sulphate, filtered and concentrated in vacuo to yield orange viscous liquids, with decreasing viscosity with increasing alkyl chain length.

POP Implants and Nanoparticle Dispersions—POP Constructs

Forming a POP Construct

Solid polymer material was ground to a fine powder and cold-compressed under 2 tons of pressure using a manual hydraulic press to form disc-shaped polymer pellets.

Formation of rod-shaped pellets with dimensions of 2 mm diameter and up to 17 mm length was achieved by vacuum compression moulding using a MeltPrep VCM Essentials instrument. Solid polymer material was ground to a fine powder and compressed under vacuum at temperatures of 30-50° C. above the glass transition temperature (T_{g}) of the polymer sample.

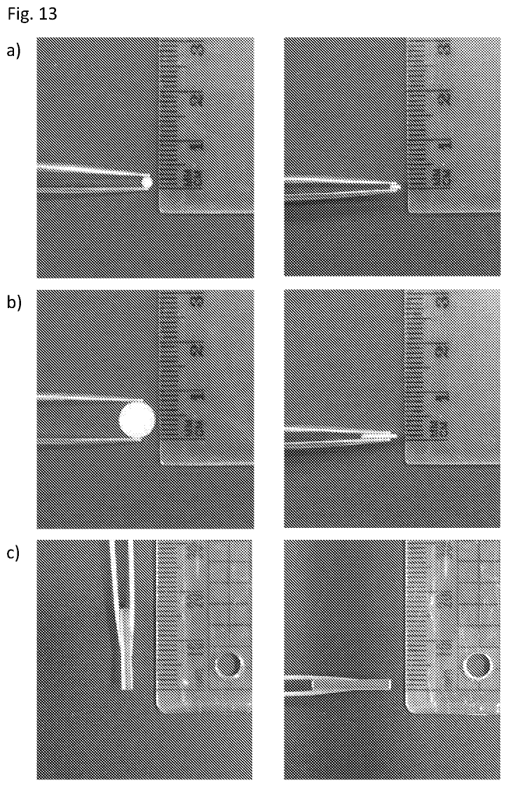

FIG. 13 shows POP constructs formed from FTC POP structures; (a) cold-compressed 2 mm disc-shaped pellet, (b) cold-compressed 7 mm disc-shaped pellet, (c) vacuum compression moulded 2 mm diameter rod-shaped pellet.

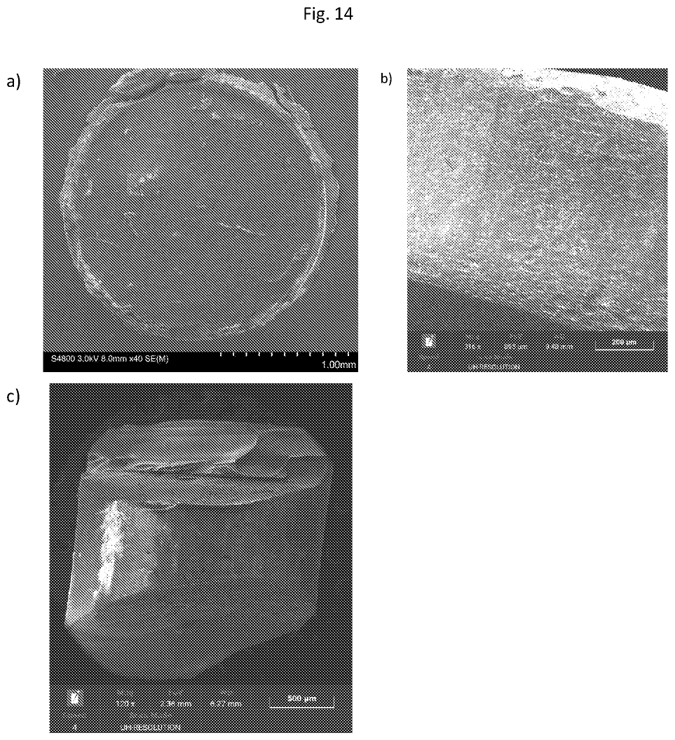

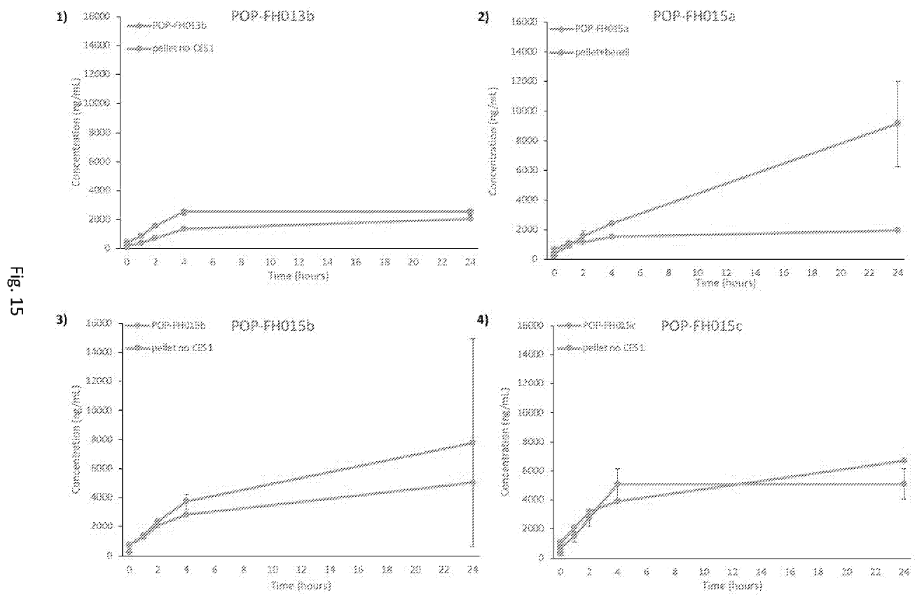

FIG. 14 shows electron microscope images of; (a) cold-compressed 2 mm disc-shaped pellet, (b) a cross-section of the cold-compressed pellet, (c) vacuum compression moulded 2 mm diameter rod-shaped pellet showing broken edges.

Pharmacological Results

Preliminary Release Studies

Preliminary studies were conducted in order to ascertain the release rate of the parent FTC from 4 POP formulations. The polymers included within these preliminary studies are listed in Table 6.

TABLE 6
Candidate polymers tested in preliminary release experiments

| | | | | | | | | | | ¹H |
|---|---|---|---|---|---|---|---|---|---|---|
| | | | | | | GPC(DMF)^{a} | | | | NMR^{b} |
| Expt no. | Monomer A | Monomer B | A:B | Solvent | Base | Mₙ | M_{w} | Ð | DPₙ | DPₙ |
| POP- | tri(ethylene glycol) | FTC | 1:1* | DCM | Pyr/DMAP | 5020 | 6210 | 1.236 | 11 | 10 |
| FH013b | bis(chloroformate) | | | | | | | | | |
| POP- | tri(ethylene glycol) | FTC | 1:1 | DCM | Pyr/DMAP | 5390 | 6310 | 1.170 | 12 | 9 |
| FH015a | bis(chloroformate) | | | | | | | | | |
| POP- | tri(ethylene glycol) | FTC | 0.95:1 | DCM | Pyr/DMAP | 4640 | 5490 | 1.182 | 10 | 9 |
| FH015b | bis(chloroformate) | | | | | | | | | |
| POP- | tri(ethylene glycol) | FTC | 0.9:1 | DCM | Pyr/DMAP | 3610 | 4250 | 1.177 | 8 | 9 |
| FH015c | bis(chloroformate) | | | | | | | | | |

^{a}DMF containing 0.01M LiBr at 60° C., 1 mL min⁻¹ flow rate.
^{b}Determined by ¹H NMR analysis in CDCl₃. [symbol]
Interfacial polycondensation.
A:B Ratios corrected for bis(chloroformate) impurity;
*92.7% purity assumed, [symbol] 97% purity assumed [symbol]
indicates data missing or illegible when filed

Release rate of FTC from the four formulations listed in the above table were assessed over 24-hour and 96-hour time periods.

24-Hour Study:

The 24-hour study included polymers containing an average of 2.8 mg of FTC per formulation per pellet. Pellet dimensions were 4 mm×4 mm×2 mm (length×width×height). Pellets were incubated within a Corning 96 well plate at 37° C., 250 rpm for 24 hours. Pellets were incubated with 100 μL microsome (125 μg/mL) containing phosphate buffer saline (PBS), containing an equivalent to 17.4 ng/mL total carboxylesterase 1 (CES1), calculated using a CES1 specific activity assay kit (Abcam, Cambridge, UK: product number: ab109717) following the manufactures protocol. Additionally, two control groups containing either pellets incubated with 100 μL of PBS or 100 μL of 1 μM benzil (CES1 inhibitor) containing PBS were included within the study. 50 μL samples were taken at 10 minutes, 30 minutes, 1 hour, 2 hours, 4 hours and 24 hours. In order to maintain sink conditions, after each sampling time point 50 μL of fresh media was added to each pellet. The FTC concentration within all samples were quantified using an adapted, previously validated liquid chromatography mass spectrometry (LCMS) method.²⁰¹

96-Hour Study:

The 96-hour study included polymers containing an average of 9.0 mg of FTC per formulation per pellet. Pellet dimensions were 7 mm×7 mm×2 mm (length×width×height). Pellets were incubated within 1.5 mL eppendorf tubes at 37° C., 250 rpm for 24 hours. Pellets were incubated with 500 μL microsome (125 μg/mL) containing phosphate buffer saline (PBS), containing an equivalent to 17.4 ng/mL total carboxylesterase 1 (CES1) quantity, calculated using a CES1 specific activity assay kit (Abcam, Cambridge, UK: product number: ab109717) following the manufactures protocol. Controls included pellets incubated with 500 μL of PBS or 500 μL of 10 μM benzil (CES1 inhibitor) containing PBS. 250 μL samples were taken at 0, 24, 48, 72 and 96 hours. In order to maintain sink conditions, after each sampling time point 250 μL of fresh media was added to each pellet. The FTC concentration within all samples were quantified using an adapted, previously validated liquid chromatography mass spectrometry (LCMS) method.²⁰¹

Results of Preliminary Release Experiments:

In Vitro Release Rate 24-Hour Study:

As shown in FIG. 15 and Table 7 below FTC release from each of the 4 polymer of prodrug formulations was higher after a 24 hour time period in comparison to control, with the exception of polymer POP-FH015c. Polymer POP-FH015a showed the highest rate of release of 0.7% of total FTC within the pellet, followed by POP-FH015b at 0.49% of total FTC released within 24 hours.

FIG. 15 shows FTC releases rate from four polymer of prodrug pellets over a 24 hour time period. [1: POP-FH013b formulation 2: POP-FH015a formulation 3: POP-FH015b formulation 4: POP-FH015c formulation. All formulations incubated with microsome containing PBS. Pellet no CES1=pellet incubated with PBS only. Pellet+benzil=pellet incubated with 10 μM benzil containing PBS. (n=3 per formulation)]

TABLE 7
Preliminary release experiments: Total FTC release
per formulation over a 24-hour time period.

| | Total FTC | Total | | FTC |
|---|---|---|---|---|
| | released over | pellet | Weight of FTC | concentration |
| POP | 24 hours | weight | per pellet | per pellet |
| formulation: | (%) | (mg) | (mg) | (μmol) |
| 013b | 0.15 | 3.2 | 1.7 | 7 |
| 013b control | 0.12 | | | |
| 015a | 0.70 | 2.4 | 1.3 | 5 |
| 015a control** | 0.15 | | | |
| 015b | 0.49 | 2.9 | 1.6 | 6 |
| 015b control | 0.31 | | | |
| 015c | 0.34 | 2.7 | 1.5 | 6 |
| 015c control | 0.44 | | | |

FTC = emtricitabine. control = pellet + PBS.
**control = pellet + PBS + microsome + benzil (10 μM)

Further Release Studies

Pharmacological studies were conducted in order to determine the release rate of parent FTC from multiple POP structures synthesised and prepared through different methods. Polymers were analysed in three forms; unprocessed polymer, cold-compressed pellet and vacuum compression moulded (VCM) pellet. The POP structures studied are outlined in Table 8.

TABLE 8
Candidate polymers included within pharmacological screening

| Polymer | | Monomer | Monomer | | GPC (DMF)^{a} | | | |
|---|---|---|---|---|---|---|---|---|
| ID | Monomer A | B | C | A:B:C | Mₙ | M_{w} | Ð | DPₙ |
| POP- | tri(ethylene glycol) | FTC | — | 1.08:1 | 4790 | 6700 | 1.398 | 11 |
| FH013b | bis(chloroformate) | | | | | | | |
| POP- | tri(ethylene glycol) | FTC | — | 1:1 | 5440 | 8160 | 1.50 | 12 |
| FH015a | bis(chloroformate) | | | | | | | |
| POP- | tri(ethylene glycol) | FTC | — | 0.95:1 | 5020 | 7170 | 1.429 | 11 |
| FH015b | bis(chloroformate) | | | | | | | |
| POP- | tri(ethylene glycol) | FTC | | 0.9:1 | 4750 | 6630 | 1.397 | 10 |
| FH015c | bis(chloroformate) | | | | | | | |
| POP- | tri(ethylene glycol) | FTC | — | 1:1 | 5880 | 8530 | 1.451 | 12 |
| FH045 | bis(chloroformate) | | | | | | | |
| ASH3.28c | aHEX | FTC | — | 1:1.05 | 3010 | 3570 | 1.180 | 7 |
| ASH4.1 | aHEX | FTC | aTMP | 0.9:1:0.06 | 2540 | 2750 | 1.08 | 4 |
| ASH4.2 | aHEX | FTC | aTMP | 0.5:1:0.16 | 2740 | 3040 | 1.11 | 4 |
| CL2-149 | tri(ethylene glycol) | isobutyl | — | 1:1 | 3690 | 6950 | 1.90 | 6 |
| | bis(chloroformate) | FTC bis- | | | | | | |
| | | MPA diol | | | | | | |

^{a}DMF containing 0.01M LiBr at 60° C., 1 mL min⁻¹ flow rate.
Mₙ—number average molecular weight,
M_{w}—weight average molecular weight,
Ð—dispersity,
DPₙ—number average degree of polymerisation

The polymers were prepared as described herein. Note: different polymer IDs denote particular monomer combinations and ratios, and experiments which are repeated or optimised under slightly different conditions, e.g. at different scales, can give different polymer characteristics. The polymers denoted by POP-FH013b, POP-FH015a, POP-FH015b and POP-FH015c in Table 6 were prepared under slightly different conditions to the polymers denoted by the same codes in Table 8 and accordingly even though the identities of monomer A and monomer B, and the ratios (allowing for purity), are the same, the polymers have slightly different characteristics, as evidenced by for example the GPC data. The variations which can be brought about by changing the conditions allow tailoring of the products and tailoring of the release characteristics. Nevertheless, the reproducibility and robustness of the polymer chemistry is evident from the similarity in the polymer results after optimisation and scale modifications, indicating significant industry utility. The following discussion and results correspond to further release studies on the products of Table 8, in contrast to the preliminary release studies on the products of Table 6.

24-Hour POP-FH013b, 015a, 015b and 015c Cold-Compressed Pellet Study:

2 mm Cold-Compressed Pellets

24 hour release rate studies of FTC from cold-compressed POP constructs of polymers POP-FH013b, 015a, 015b and 015c with dimensions of 2 mm diameter×1 mm height and average FTC mass of 1.6 mg with a total pellet mass of 2.8 mg were conducted. Pellets were incubated within a Corning 96 well plate at 37° C., 250 rpm for 24 hours. Pellets were incubated with 100 μL microsome (125 μg/mL) containing phosphate buffer saline (PBS), containing an equivalent to 17.4 ng/mL total carboxylesterase 1 (CES1), calculated using a CES1 specific activity assay kit (Abcam, Cambridge, UK: product number ab109717) following the manufactures protocol. Additionally, two control groups containing either pellets incubated with 100 μL of PBS or 100 μL of 1 μM benzil (CES1 inhibitor) containing PBS and 125 mg/mL microsome were included within the study. 50 μL samples were taken at 10 minutes, 30 minutes, 1 hour, 2 hours, 4 hours and 24 hours. In order to maintain sink conditions, after each sampling time point 50 μL of fresh media was added to each pellet. The FTC concentration within all samples were quantified using an adapted, previously validated liquid chromatography mass spectrometry (LCMS) method.²⁰¹

72-Hour POP-FH013b, 015a, 015b and 015c Cold-Compressed Pellet Study.

7 mm Cold-Compressed Pellets

72 hour release rate studies of FTC from cold-compressed POP constructs of polymers POP-FH013b, 015a, 015b and 015c with dimensions of 7 mm diameter×0.5 mm height and average FTC mass of 9 mg with a total pellet mass of 16 mg were conducted. Pellets were incubated within 1.5 mL eppendorf tubes at 37° C., 250 rpm for 72 hours. Pellets were incubated with 1 mL microsome (125 μg/mL) diluted in phosphate buffer saline (PBS), containing an equivalent to 17.4 ng/mL total carboxylesterase (CES) 1 quantity, calculated using a CES1 specific activity assay kit (Abcam, Cambridge, UK: product number: ab109717) following the manufacturers protocol. Controls included pellets incubated with 1 mL of PBS or 1 mL of 1 mM benzil (CES1 inhibitor), dissolved in 0.1% DMSO, 4% MeOH containing PBS and 125 μg/mL of microsome. 500 μL samples were taken at 0, 24, 48 and 72 hours. In order to maintain sink conditions, after each sampling time point 500 μL of fresh PBS, microsome or benzyl and microsome containing solution was added to each pellet as appropriate. The FTC concentration within all samples were quantified using an adapted, previously validated liquid chromatography mass spectrometry (LCMS) method.

72-Hour POP-FH045 Unprocessed Polymer, Cold-Compressed Pellet and Vacuum Compression Moulded Pellet Study:

To study the in vitro FTC release from polymer POP-FH045 over 72 hours, three forms of the polymer were studied in order to better understand how polymer processing related to FTC release. The average total mass of polymer within the studied samples of unprocessed polymer, cold-compressed pellet and vacuum compression moulded (VCM) pellets was 9 mg, containing an average FTC mass of 5 mg. Cold-compressed pellet dimensions were 4 mm diameter×1 mm height and dimensions for the VCM pellets were 2 mm diameter×2 mm height. The studied conditions and methods utilised were identical to those outlined within the 72-hour POP-FH013b, 015a, 015b and 015c cold-compressed pellet study, with the exception of the unprocessed polymer which had an individual sample for each time point, in order to maintain the experimental conditions throughout the study at each time point.

72-Hour ASH4.1, ASH4.2 and ASH3.28c Unprocessed Polymer and Vacuum Compression Moulded Pellet Study:

The in vitro release of FTC from polymers ASH4.1, ASH4.2 and ASH3.28c over 72 hours was determined through conducting the same experimental procedure outlined within the 72-hour POP-FH045 unprocessed polymer, cold-compressed pellet and vacuum compression moulded pellet study methodology. Both unprocessed polymers and VCM pellets were included within the study for all three POP structures. For the VCM pellets the average total mass of ASH3.28c polymer was 9.3 mg containing an average FTC mass of 4.9 mg. For ASH4.1 the average total mass was 8.1 mg containing an average of 4.9 mg of FTC. Finally for ASH4.2 the average total polymer mass per sample was 8.5 mg containing an average of 4.9 mg of FTC. Melt preparation pellet dimensions were 2 mm diameter×2 mm height. Within the unprocessed polymer studies for all three polymers a total of 9 mg of unprocessed polymer was studied per sample per time point.

72-Hour CL2-149 Unprocessed Polymer Study:

To determine the in vitro FTC release from polymer CL2-149 over 72 hours the same experimental conditions were applied as previously described within the 72-hour POP-FH045 unprocessed polymer, cold-compressed pellet and vacuum compression moulded pellet study for the study of the unprocessed polymers. Within the experiment the average total mass of polymer within each sample was 13.5 mg containing an average FTC mass of 5.0 mg.

Results:

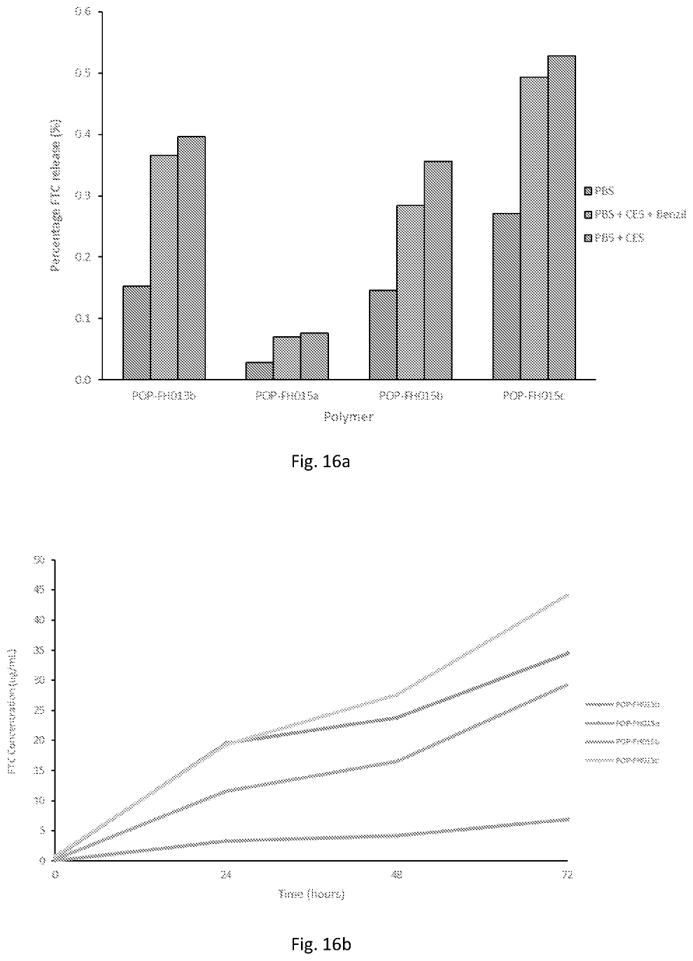

72-hour POP-FH013b, 015a, 015b and 015c cold-compressed pellet study: FIG. 16a shows the percentage of total FTC released over 72 hours from POP-FH013b, POP-FH015a, POP-FH015b and POP-FH015c cold-compressed pellets. FIG. 16b shows total FTC released from POP-FH013b, 015a, 015b and 015c cold-compressed pellets over 72 hours when exposed to PBS+CES.

As shown in FIG. 16a FTC release from each of the four POP structures was higher after 72 hours in comparison to control. Polymer POP-FH015c showed the highest FTC release of 0.5% of total FTC followed by POP-FH015b at 0.4%. As shown in FIG. 16b all four POP structures showed continuous release over the 72 hour time period with the highest FTC concentration at 72 hours observed for POP-FH015c at 44.2 μg/mL and the second POP-FH013b at 34.5 μg/mL.

72-Hour POP-FH045 Unprocessed Polymer, Cold-Compressed Pellet and Vacuum Compression Moulded Pellet Study:

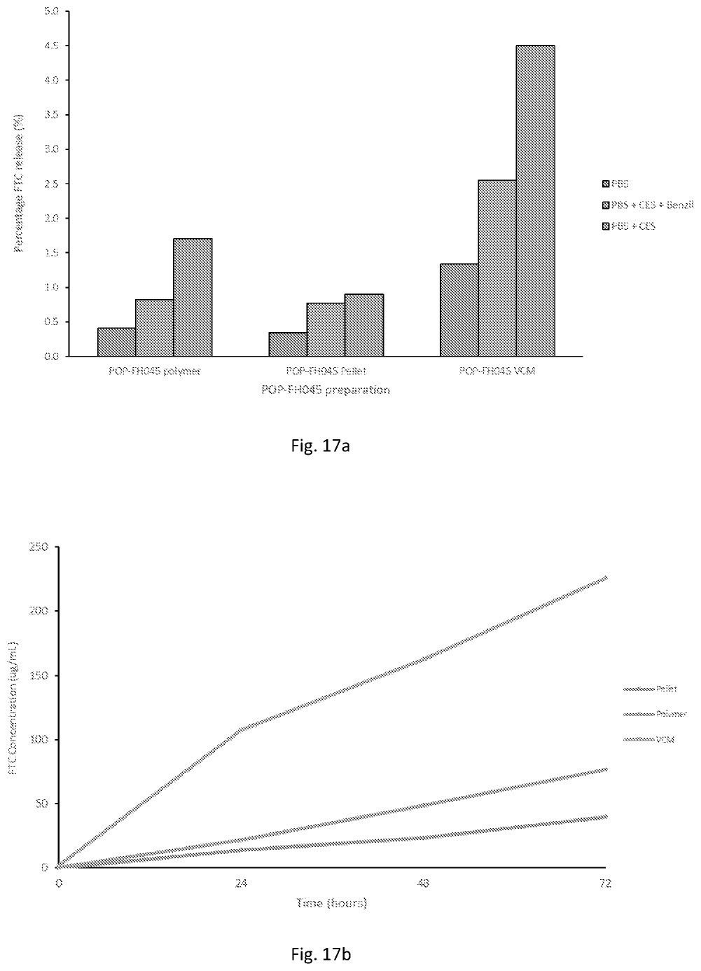

FIG. 17a shows the percentage of total FTC released over 72 hours from POP-FH045 preparations. FIG. 17b shows total FTC released from three POP-FH045 preparations over 72 hours when exposed to PBS+CES.

As shown in FIG. 17a FTC release from each of the POP-FH045 polymers was higher after 72 hours in comparison to control. The melt preparation pellet showed the highest percentage release of FTC of 4.5% of total FTC followed by the non-pressed polymer at 1.7%. As shown in FIG. 17b all of the POP structures showed continuous FTC release over the 72 hour time period with the highest FTC concentration at 72 hours observed for the melt preparation pellet at 225.8 μg/mL followed by the non-pressed polymer at 76.7 μg/mL and the pressed pellet at 39.9 μg/mL.

72-Hour ASH4.1, ASH4.2 and ASH3.28 Unprocessed Polymer, Cold-Compressed Pellet and Vacuum Compression Moulded Pellet Study:

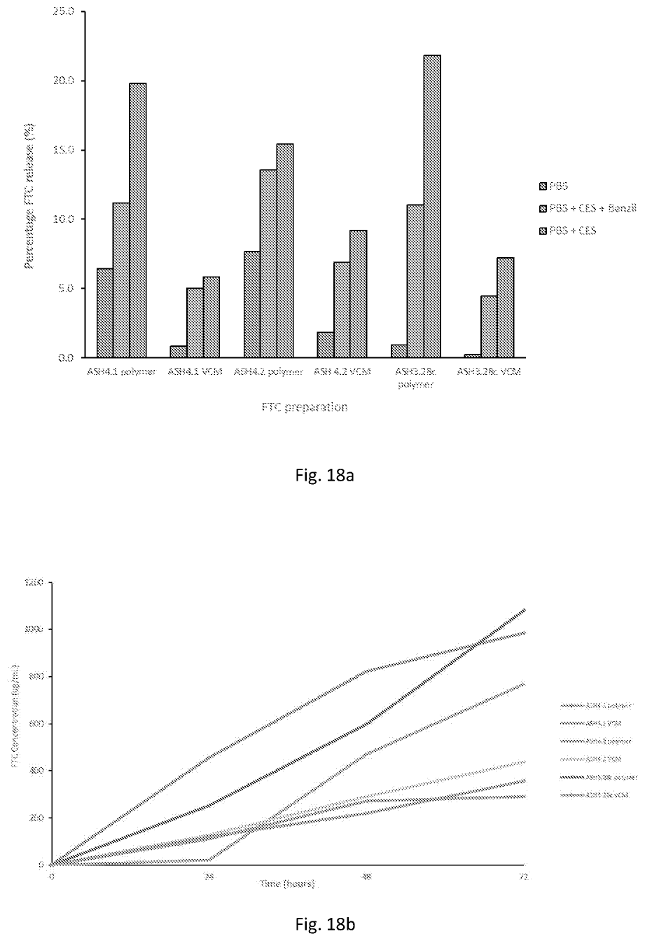

FIG. 18a shows the percentage of total FTC released over 72 hours from ASH4.1, 4.2 and 3.28c unprocessed polymer and VCM pellet. FIG. 18b shows total FTC released from ASH 4.1, 4.2 and 3.28c unprocessed polymer or VCM pellet over 72 hours when exposed to PBS+CES.

As shown in FIG. 18a FTC release from each of the polymer preparations was higher after 72 hours in comparison to control. The unprocessed polymers showed higher total FTC release of 19.8%, 15.4% and 21.8% for POP structures ASH 4.1, 4.2 and 3.28c respectively, in comparison to that seen for the VCM pellets at 5.9%, 9.2% and 7.2% respectively. Additionally as shown in FIG. 18b all of the preparations studied showed continuous release over the 72 hour time period with the highest FTC concentration at 72 hours observed for the unprocessed ASH3.28c polymer at 1080.6 μg/mL followed by the unprocessed ASH4.2 polymer at 985.3 μg/mL.

72-Hour CL2-149 Unprocessed Polymer Study:

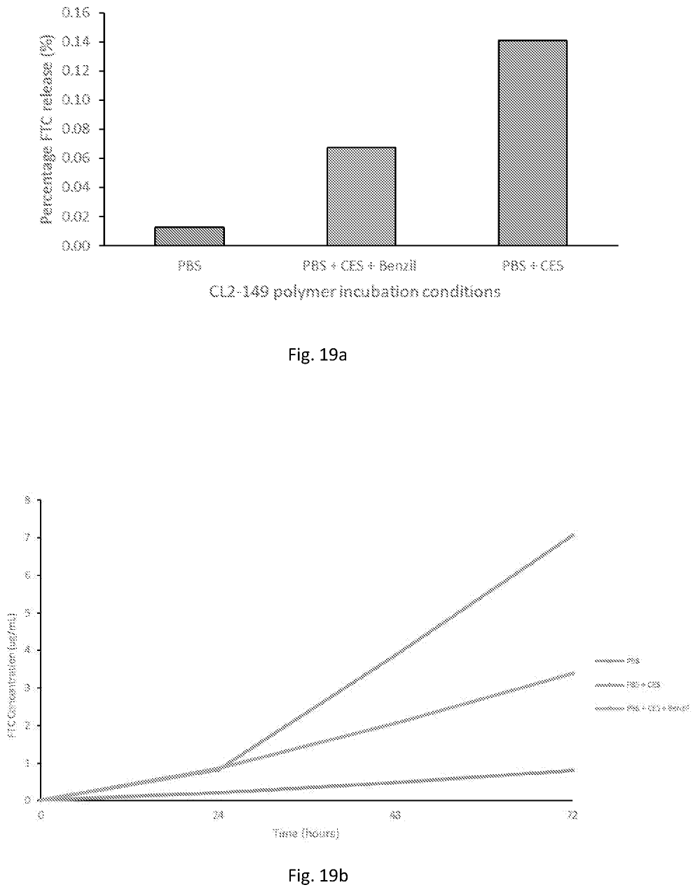

FIG. 19a shows the percentage of total FTC released over 72 hours from unprocessed CL2-149 polymer incubated with CES containing PBS, benzil containing PBS and PBS alone. FIG. 19b shows total FTC released from unprocessed CL2-149 polymer over 72 hours under all conditions

As shown in FIG. 19a FTC release from the unprocessed CL2-149 polymer was higher after 72 hours in comparison to control with a 0.14% of total FTC released in comparison to 0.01% from PBS exposed polymer. As shown in FIG. 19b the POP structure showed continuous release over the 72 hour time period with the highest FTC concentration at 72 hours observed at 0.14 μg/mL.

Galleria mellonella studies:

In order to determine the release rate of FTC within a non-mammalian animal model, studies within Galleria mellonella (G. mellonella) were conducted. Several optimisation studies were completed, the first focusing on pellet size selection, followed by a dose optimisation study and a 96-hour release rate study, before extended release over 30 days studied for lead candidate formulations. The FTC concentration within all samples was quantified using an adapted, previously validated liquid chromatography mass spectrometry (LCMS) method.²⁰¹

Pellet Size Optimisation Study:

G. mellonella were selected within a 300-400 mg weight range. All groups were fasted and incubated for 3 days at 1-5° C. prior to study initiation. At study day 0, pellets were implanted into the G. mellonella via the lower left proleg using a specially designed applicator. Three pellet sizes were investigated of the following dimensions: 1 mm×1 mm×2 mm, 2 mm×2 mm×2 mm and 4 mm×4 mm×2 mm (length×width×height). Each pellet contained an average of 3 mg of FTC per pellet per formulation. This study is a serial sacrifice design, with culls completed at 0, 24, 48 and 96 hours. The terminal endpoint was 96 hours, or at the point when all insects were deceased. At each time point, each study group was incubated at 1-5° C. for 10 minutes before haemolymph was extracted and pooled using a previously defined method.⁰² The optimum pellet size was selected based on mortality rate within each study group, ease of loading into the implant applicator and the rate of FTC release per pellet in comparison to the in vitro release rate study results.

Dose Optimisation Study:

A toxicology study was conducted in order to ascertain the maximum dose of FTC per pellet that can be implanted within the G. mellonella. This study was serial sacrifice design, with culls completed at 0, 24, 48 and 96 hours. The terminal endpoint was 96 hours, or at the point when all insects were deceased. FTC pellet dimensions were selected based on the pellet optimisation study. Weight of FTC within each pellet increased (for example 5 mg, 10 mg, 20 mg) until a lethal dose was established. All G. mellonella were selected and housed as outlined above. At each time point haemolymph was extracted and FTC release per pellet was quantified as outlined above. Optimum dose was selected based on mortality rate, and the rate of FTC release per pellet in comparison to the in vitro release rate study results.

96-Hour Release Study:

Once pellet dimensions and dose were selected a 96-hour release rate study was completed within the G. mellonella for all polymer candidates. This study utilised the same G. mellonella selection, housing, implantation and serial sacrifice conditions as stated above.

30-Day Study:

Lead candidate formulations, selected based on the release rate data obtained during the 96-hour release rate study, were entered into a 30-day study within G. mellonella. The same selection, housing, implantation and serial sacrifice methods were used as stated previously. Culls were completed at day 0 followed by every 3 days, with the terminal endpoint for the study being 30 days, or at the point when all insects were deceased.

Procedures and Characterization for Synthesis of Tenofovir Alafenamide (TAF) Conjugates/Prodrugs

Toward preparation of trimethyl lock (TML)-containing B₂ monomers, the synthesis of TML-TAF conjugates has been explored. Conjugates containing azido and alkyne groups can be clicked to generate model N,N-linked TAF POP fragments to study activation relevant to FIG. 6.

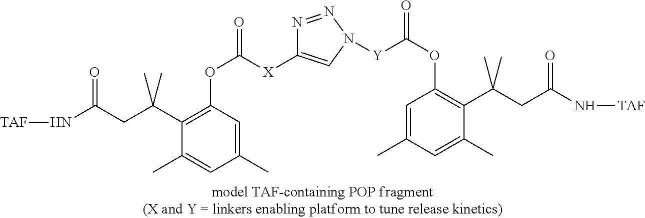

Alternatively, clickable TML-TAF conjugates can be further modified through click chemistry to incorporate a diol, producing A2 monomers for a pendant strategy 3, FIG. 1.

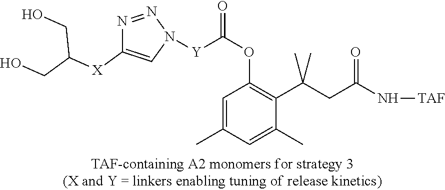

### Synthesis of Trimethyl Lock Intermediates

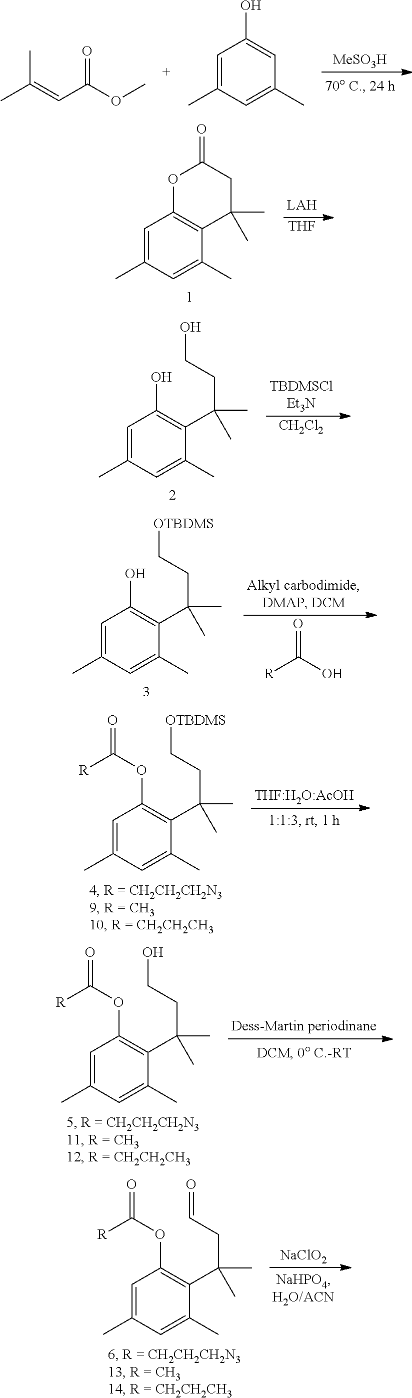

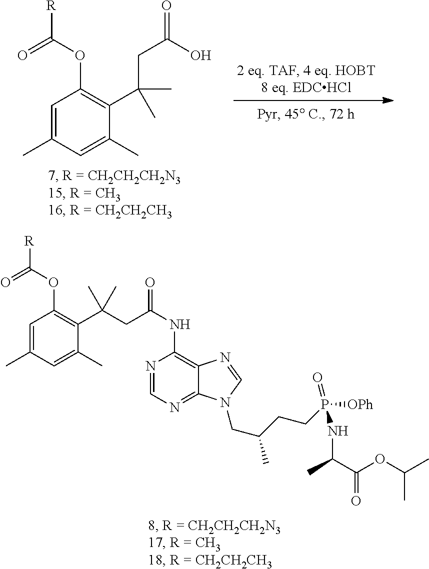

Synthesis of 4,4,5,7-tetramethylchroman-2-one (1):³⁰¹ To a dry round-bottom flask containing methane sulphonic acid (24 mL), 3,5-dimethyl phenol (20 g, 163.7 mmol) was added and stirred followed by addition of dimethyl acrylic acid (20.56 g, 180 mmol) generating a viscous mixture. The mixture was heated to 70° C. and stirred for 24 hours resulting in a black colored viscous solution. The reaction mixture was poured onto ice and the product was extracted using ethyl acetate (500 mL). The ethyl acetate layer was separated, washed with saturated sodium bicarbonate solution (250 mL) and saturated sodium chloride solution (250 mL). The organic layer was dried over magnesium sulfate, filtered and concentrated in vacuo giving a yellow suspension. Hexanes (250 mL) was added to the suspension, which was stirred at ambient temperature for 16 hours to obtain a white suspension. The suspension was filtered, yielding a white solid (26 g, 78%; 1^{st} crop 22.6 g, 2^{nd} crop 3.4 g). 1H NMR (500 MHz, CDCl₃) δ ppm 6.75 (s, 2H), 2.6 (s, 2H), 2.47 (s, 3H), 2.28 (s, 3H), 1.44 (s, 6H).

Synthesis of 2-(4-Hydroxy-2-methylbutan-2-yl)-3,5-dimethylphenol (2):³⁰¹ To a dry round-bottom flask flushed with argon, 2.4 M lithium aluminium hydride solution in THF (92 mL, 220.8 mmol) was added and cooled to −30° C. under argon. In a separate dry round bottom flask, compound 1 (23 g, 112.6 mmol) was dissolved in anhydrous THF (100 mL) and added dropwise over 1 hour to the flask containing lithium aluminium hydride solution at −30° C. under argon atmosphere. The reaction mixture was stirred for 16 hours allowing the temperature to gradually rise to ambient temperature. The reaction mixture was cooled in an ice-bath (0-4° C.) and quenched by adding Na₂SO₄.10H₂O whilst maintaining the temperature. The reaction mixture was filtered over celite and concentrated in vacuo to obtain an off-white solid (22.5 g, 96%). ¹H NMR (500 MHz, CDCl₃) δ ppm 6.5 (s, 1H), 6.34 (s, 1H), 5.86 (br s, 1H), 3.65-3.62 (t, 2H, J=7.5 Hz), 2.48 (s, 3H), 2.26-2.23 (t, 2H, J=7.5 Hz), 2.43 (s, 3H), 1.56 (s, 6H).

Synthesis of 2-[4-[(et-Butyldimethylsilyl)oxy]-2-methylbutan-2-yl]-3,5-dimethylphenol (3):³⁰¹ In a dry round-bottom flask containing compound 2 (22.5 g, 108.02 mmol) and tert-butyldimethylsilyl chloride (17.91 g, 118.82 mmol) methylene chloride (200 mL) was added to generate a suspension. The suspension was cooled in an ice bath (0-4° C.), and triethylamine (60.2 mL, 432.08 mmol) was added dropwise over 1 hour. The ice-bath was removed after stirring the suspension for 1 hour. The reaction mixture was stirred for 16 hours during which the temperature rose to ambient temperature. The reaction was quenched by adding H₂O (200 mL) followed by separating the organic layer, washing it with 10% citric acid (200 mL), saturated sodium chloride solution (200 mL) and drying it over magnesium sulfate. The organic layer was filtered and dried in vacuo to obtain a suspension of white solid in pink-brown liquid. Hexanes (200 mL) was added to the suspension, the mixture cooled to −20° C. for 1 hour and filtered to get product as a white solid (20 g). A second crop of was obtained from the filtrate (8 g). The two solids were combined to get the product as a white solid (28 g, 80%). ¹H NMR (500 MHz, CDCl₃) δ ppm 6.5 (s, 1H), 6.41 (s, 1H), 5.61 (s, 1H), 3.6 (m, 2H), 2.46 (s, 3H), 2.19 (s, 3H), 2.12 (m, 2H), 1.56 (s, 6H), 0.88 (s, 9H), 0.02 (s, 6H).

General procedure for synthesis of TML Intermediates 4, 9-10:³⁰¹ In a dry round-bottom flask, a solution of compound 3 (1 eq.), the appropriate N,N′-alkylcarbodiimide (2 eq.) and 4-dimethylaminopyridine (3 eq.) in methylene chloride (25 mL) was cooled in an ice bath (0-4° C.). The appropriate acid (2 eq.) was added to the cooled solution and the mixture was stirred for 16 hours during which the reaction mixture warmed to ambient temperature. The reaction mixture was filtered and purified via silica flash chromatography (ethyl acetate in hexanes).

General procedure for synthesis of TML Intermediates 5, 11-12:³⁰¹ In a round-bottom flask compound (4, 9-10, 1 eq.) was dissolved in a mixture of acetic acid:H₂O:THF (3:1:1) and stirred at ambient temperature for 2 hours. The solvents were removed in vacuo, and the product purified via silica flash chromatography (ethyl acetate in hexanes). The product was obtained as a viscous liquid.

General procedure for synthesis of TML Intermediates 6, 13-14:³⁰¹ In a dry round-bottom flask compound (5, 11-12, 1 eq.) was dissolved in anhydrous methylene chloride and cooled in an ice-bath (0-4° C.) under argon atmosphere. Dess-Martin periodinane (1,1,1-tris(acetyloxy)-1,1-dihydro-1,2-benziodoxol-3-(1H)-one, 2-3 eq.) was added, and the reaction suspension stirred for 16 hours during which the reaction mixture warmed to ambient temperature. To the reaction suspension, NaHCO₃ and Na₂S₂O₃ were added and the mixture was stirred for 30 min. Water was added to the suspension and the layers were separated. The organic layer was washed with H₂O, saturated sodium chloride solution and dried over magnesium sulfate. The organic phase was filtered, concentrated in vacuo, and the product purified via silica flash chromatography (ethyl acetate in hexanes). The product was obtained as a viscous liquid.

General procedure for synthesis of TML Intermediates 7, 15-16:³⁰¹ In a round-bottom flask compound (6, 13-14, 1 eq.) and NaH₂PO₄ (1 eq.) were dissolved in acetonitrile:H₂O (2.5:1). The solution was cooled in an ice bath (0-4° C.), and a solution of NaClO₂ (2.5 eq.) in H₂O (10 mL) was added over 10 min. The reaction mixture was stirred for 1 hour at 0-4° C., then at ambient temperature for 30 min. The reaction was quenched by adding Na₂S₂O₃ (3 eq.). Acetonitrile was removed in vacuo and the pH of the residual aqueous suspension was adjusted to 1-2 using 1M HCl. The product was extracted in ethyl acetate. The organic phase was washed with H₂O, saturated sodium chloride solution, and dried over magnesium sulfate. The organic phase was filtered, concentrated in vacuo, and the product purified via silica flash chromatography (ethyl acetate in hexanes). The product was obtained as a viscous liquid.

General procedure for TML acid-TAF amide coupling (Compounds 8, 17, 18):³⁰¹ In a round-bottom flask acid (1 eq. 7, 17, 18), tenofovir alafenamide (2 eq.), hydroxybenzotriazole (4 eq.), N—N′-dialkylcarbodiimide (8 eq.) and pyridine (solvent) were stirred at ambient temperature for 0.5-2 hours and then at 40-45° C. for 72 hours. Solvent was removed in vacuo, the residue was dissolved in methylene chloride and washed with H₂O. The layers were separated and the aqueous layer extracted with methylene chloride. The organic layers were combined, washed with saturated sodium chloride solution and dried over magnesium sulfate. The organic phase was filtered, concentrated in vacuo and the product purified via silica flash chromatography (0-100% ethyl acetate in hexanes, 0-20% methanol in methylene chloride). The product was obtained as a viscous liquid.

Alternative procedure for TML acid-TAF amide coupling (1):³⁰¹ In a dry round-bottom flask 1 eq. acid, 1.1 eq. HATU (1-[bis(dimethylamino)methylene]-1H-1,2,3-triazolo[4,5-b]pyridinium 3-oxid hexafluorophosphate, N-[(dimethylamino)-1H-1,2,3-triazolo-[4,5-b]pyridin-1-ylmethylene]-N-methylmethanaminium hexafluorophosphate N-oxide), were dissolved in methylene chloride. Anhydrous alkyl amine (2 eq.) was added and the reaction mixture stirred for 5 minutes to 2 hours followed by addition of tenofovir alafenamide (0.25-1 eq). The resulting mixture was stirred for 16-96 hours. The reaction mixture was purified via silica flash chromatography (0-100% ethyl acetate in hexanes, 0-20%, methanol in methylene chloride) to obtain product as an amorphous solid or viscous liquid.

Alternative procedure for TML acid-TAF amide coupling (II):³⁰² In a dry round-bottom flask 1 eq. acid and 3 eq. organic base (N-methyl imidazole) were dissolved in methylene chloride and cooled on an ice bath (0-4° C.). Methanesulfonyl chloride (1-3 eq.) was added and the reaction mixture was stirred for 15 minutes to 2 hours followed by addition of tenofovir alafenamide (0.25-1 eq.). The resulting reaction mixture was stirred for 16-96 hours. The reaction mixture was purified via silica flash chromatography (0-100% ethyl acetate in hexanes, 0-20%, methanol in methylene chloride) to obtain product as an amorphous solid or viscous liquid.

Synthesis of 2-(4-((tert-butyldimethylsilyl)oxy)-2-methylbutan-2-yl)-3,5-dimethylphenyl 4-azidobutanoate (4):³⁰¹ In a dry round-bottom flask, a solution of compound 3 (1 g, 3.1 mmol), 4-azidobutyric acid (0.8 g, 6.2 mmol) and 4-dimethylaminopyridine (1.13 g, 9.3 mmol) in methylene chloride (25 mL) was cooled on an ice-bath (0-4° C.). N,N′-Diisopropylcarbodiimide (0.96 mL, 6.2 mmol) was added to the cooled solution and the mixture was stirred for 16 hours during which the reaction mixture warmed to ambient temperature. The reaction mixture was filtered and purified via silica flash chromatography (0-10% ethyl acetate in hexanes). The product was obtained as a viscous liquid (1.2 g, 89%). 1H NMR (500 MHz, CDCl₃) δ ppm 6.82 (s, 1H), 6.54 (s, 1H), 3.5-3.44 (m, 4H), 2.67-2.64 (t, 2H, J=7.5 Hz), 2.53 (s, 3H), 2.24 (s, 3H), 2.05-2.01 (m, 4H), 1.47 (s, 6H), 0.86 (s, 9H), 0.02 (s, 6H).

Synthesis of 2-(4-hydroxy-2-methylbutan-2-yl)-3,5-dimethylphenyl 4-azidobutanoate (5):³⁰¹ In a round-bottom flask compound 4 (2.2 g, 5.07 mmol) was dissolved in a mixture of acetic acid (30 mL), H₂O (10 mL), THF (10 mL) and stirred at ambient temperature for 2 hours. The solvents were removed in vacuo and the product purified via silica flash chromatography (0-50% ethyl acetate in hexanes). The product was obtained as a viscous liquid (0.8 g, 49%). ¹H NMR (500 MHz, CDCl₃) δ ppm 6.84 (s, 1H), 6.55 (s, 1H), 3.56-3.53 (t, 2H, J=7.5 Hz), 3.47-3.45 (t, 2H, k=5 Hz), 2.69-2.66 (t, 2H, J=7.5 Hz), 2.54 (s, 3H), 2.24 (s, 3H), 2.07-2.00 (m, 4H), 1.50 (s, 6H).

Synthesis of 3,5-dimethyl-2-(2-methyl-4-oxobuten-2-yl)phenyl 4-azidobutanoate (6):³⁰¹ In a dry round-bottom flask compound 5 (600 mg, 1.87 mmol) was dissolved in anhydrous methylene chloride (30 mL) and cooled on an ice-bath (0-4° C.) under argon atmosphere. Dess-Martin periodinane (1,1,1-tris(acetyloxy)-1,1-dihydro-1,2-benziodoxol-3-(1H)-one, 1.6 g, 3.75 mmol) was added and the reaction suspension was stirred for 16 hours during which the reaction mixture warmed to ambient temperature. The reaction mixture was filtered over celite, then stirred with NaHCO₃ (2.5 g) and Na₂S₂O₃ (2.5 g). Water (50 mL) was added to the suspension and the layers were separated. The organic layer was washed with H₂O (50 mL), saturated sodium chloride solution in H₂O (50 mL), and dried over magnesium sulfate. The organic phase was filtered, concentrated in vacuo and the product purified via silica flash chromatography (0-50% ethyl acetate in hexanes). The product was obtained as a viscous liquid (500 mg, 84%). 1H NMR (500 MHz, CDCl₃) δ ppm 9.55 (s, 1H), 6.86 (s, 1H), 6.59 (s, 1H), 3.47-3.45 (t, 2H, J=5 Hz), 2.82 (s, 2H), 2.69-2.66 (t, 2H, J=7.5 Hz), 2.55 (s, 3H), 2.25 (s, 3H), 2.06-2.00 (m, 2H), 1.57 (s, 6H).

Synthesis of 3-(2-((4-azidobutanoyl)oxy)-4,6-dimethylphenyl)-3-methylbutanoic acid (7):³⁰¹ In a round-bottom flask compound 6 (500 mg, 1.57 mmol) and NaH₂PO₄ (130.4 mg, 0.945 mmol) were dissolved in acetonitrile (10 mL) and H₂O (4 mL). The solution was cooled in an ice-bath (0-4° C.) and a solution of NaClO₂ (4.92 mmol) in H₂O (10 mL) was added over 10 min. The reaction mixture was stirred for 1 hour at 0-4° C., then at ambient temperature for 30 min. The reaction was quenched with Na₂S₂O₃ (2.5 g). Acetonitrile was removed in vacuo and the pH of the residual aqueous suspension was adjusted to 1-2 using 1M HCl. The product was extracted with ethyl acetate (50 mL), washed with H₂O (20 mL), saturated sodium chloride solution (20 mL) and dried over magnesium sulfate. The organic phase was filtered, concentrated in vacuo and the product purified via silica flash chromatography (0-50% ethyl acetate in hexanes). The product was obtained as a viscous liquid (321.16 mg, 61%). ¹H NMR (500 MHz, CDCl₃) δ ppm 6.83 (s, 1H), 6.58 (s, 1H), 3.46-3.44 (t, 2H, J=5 Hz), 2.83 (s, 2H), 2.70-2.67 (t, 2H, J=7.5 Hz), 2.54 (s, 3H), 2.24 (s, 3H), 2.05-1.99 (m, 2H), 1.58 (s, 6H).

Synthesis of Compound 8:³⁰¹ In a round-bottom flask compound 7 (400 mg, 1.19 mmol), tenofovir alafenamide (1.14 g, 2.39 mmol), hydroxybenzotriazole (653 mg, 4.79 mmol), N-(3-dimethylaminopropyl)-N′-ethylcarbodiimide hydrochloride (EDC, 1.84 g, 9.59 mmol) and pyridine (25 mL) were stirred at ambient temperature for 2 hours under argon and then at 40-45° C. for 72 hours. Pyridine was removed in vacuo, the residue was dissolved in methylene chloride (100 mL) and washed with H₂O (50 mL). The layers were separated, and the aqueous layer extracted with methylene chloride (100 mL). The organic layers were combined, washed with saturated sodium chloride solution (100 mL) and dried over magnesium sulfate. The organic phase was filtered, concentrated in vacuo and the product purified via silica flash chromatography (0-100% ethyl acetate in hexanes, 0-10% methanol in methylene chloride). The product was obtained as a viscous liquid (290 mg, 31%). ¹H NMR (500 MHz, CDCl₃) δ ppm 8.66 (s, 1H), 8.47 (br s, 1H), 8.12 (s, 1H), 7.2-7.16 (t, 2H, J=10 Hz), 7.10-7.07 (t, 1H, J=7.5 Hz), 6.97-6.96 (d, 1H, J=5 Hz), 6.74 (s, 1H), 6.55 (s, 1H), 5.03-4.96 (m, 1H), 4.38-4.35 (d, 1H, J=15 Hz), 4.17-4.13 (dd, 1H, J=5 Hz, J=15 Hz), 4.07-3.99 (m, 1H), 3.94-3.87 (m, 2H), 3.7-3.63 (m, 2H), 3.40-3.38 (t, 2H, J=5 Hz), 3.26-3.25 (b d, 2H, J=5 Hz), 2.67-2.64 (t, 2H, J=7.5 Hz), 2.54 (s, 3H), 2.17 (s, 3H), 2.00-1.95 (m, 2H), 1.66 (s, 6H), 1.29-1.28 (d, 3H, J=5 Hz), 1.23-1.21 (t, 9H, J=5 Hz). ³¹P NMR (500 MHz, CDCl₃) δ ppm 20.65 (s). ESI MS: m/z calculated for C₃₉H₅₀N₉O₈P (M⁺+H⁺) 792.36. Found 792.44.

Synthesis of Compound 18:³⁰³ In a dry round-bottom flask 16 (360 mg, 1.23 mmol), HATU (1-[bis(dimethylamino)methylene]-1H-1,2,3-triazolo[4,5-b]pyridinium 3-oxid hexafluorophosphate, N-[(dimethylamino)-1H-1,2,3-triazolo-[4,5-b]pyridin-1-ylmethylene]-N-methylmethanaminium hexafluorophosphate N-oxide, 515 mg, 1.35 mmol) were dissolved in methylene chloride under argon. Anhydrous N,N-diisopropylethylamine (428 μL, 2.46 mmol) was added and the reaction mixture stirred for 30 minutes. Tenofovir alafenamide (293.1 mg, 0.615 mmol) was added and the reaction mixture stirred for 144 hours at ambient temperature. The reaction mixture was purified via silica flash chromatography (0-100% ethyl acetate in hexanes, 0-20% methanol in methylene chloride) to obtain product as viscous syrup (98.1 mg, 21%). ¹H NMR (500 MHz, CDCl₃) δ ppm 8.66 (s, 1H), 8.39 (br s, 1H), 8.12 (s, 1H), 7.21-7.18 (t, 2H, J=7.5 Hz), 7.11-7.08 (t, 1H, J=7.5 Hz), 6.98-6.97 (d, 1H, J=5 Hz), 6.73 (s, 1H), 6.57 (s, 1H), 5.05-4.97 (m, 1H), 4.39-4.36 (d, 1H, J=15 Hz), 4.18-4.14 (dd, 1H, J=5 Hz, J=15 Hz), 4.08-4.00 (m, 1H), 3.94-3.89 (m, 2H), 3.68-3.63 (m, 2H), 3.61-3.57 (t, 1H, J=10 Hz), 3.24-3.17 (b t, 2H), 2.57-2.54 (m, 5H), 2.17 (s, 3H), 1.82-1.74 (m, 2H), 1.71 (s, 3H), 1.68 (s, 6H), 1.31-1.30 (d, 3H, J=5 Hz), 1.25-1.22 (t, 9H, J=7.5 Hz), 1.03-1.00 (t, 3H, J=7.5 Hz). ³¹P NMR (500 MHz, CDCl₃) δ ppm 20.61 (s).

General Procedure for Hydrolysis Measurements of TAF-TML Conjugates:

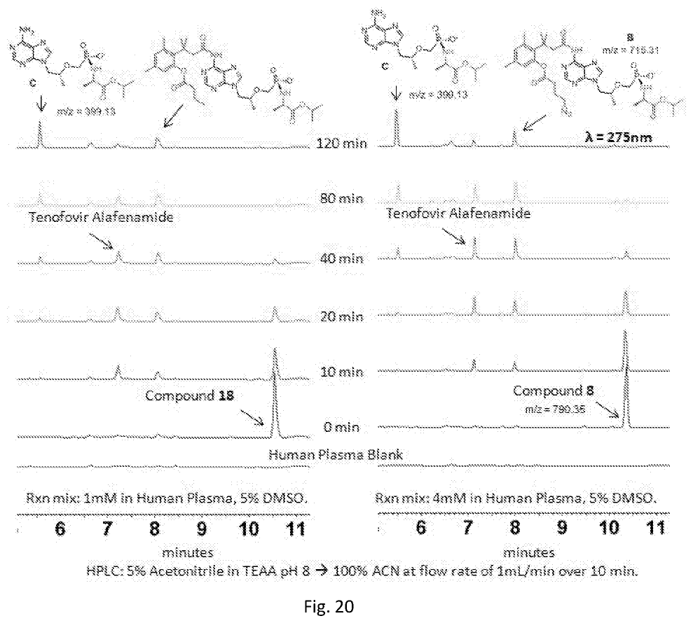

To evaluate activation of TAF-TML conjugates, and with reference to FIG. 20, HPLC coupled with UV detection was used with the following method: 5% to 100% B over 10 minutes at a flow rate of 1 mL min⁻¹ (solvent A: Et₃NHOAc (50 mM, pH 8), solvent B: acetonitrile. Human plasma (Bioreclamation) was preincubated at 37° C. for 5 minutes. The TAF-TML prodrug (1 mM or 4 mM) was added and the reaction was incubated at 37° C. Aliquots were taken at each time point and quenched in three volumes of ice-cold methanol. The quenched aliquots were then centrifuged at 14000 rpm for 10 min. The supernatant was diluted in nine volumes of Tris buffer (100 mM, pH 7.4) and injected onto the HPLC for analysis.

Analysis of TAF-TML conjugates (8 and 18) reveal the cleavage of the TAF-TML amide bond to release TAF. Release of the drug (TAF) is accompanied by cleavage of phenol ester. Within 2 hours, the TAF-TML conjugate (8,18) disappears.

Hydrolysis Profile of TAF-TML Conjugates in Pooled Mixed Gender Human Plasma. TAF-TML Conjugate Undergoes Activation by Two Pathways to Release TAF, Desphenyl TAF, and Desphenyl Analogues of the TAF-TML Conjugates.^{304,305}

TAF-TML conjugates metabolize in pooled mixed gender human plasma via two different pathways, which involve cleavage of TML esters and cleavage of phenol from the phosphonamidate moiety. The cleavage of the TML ester triggers cyclization that leads to cleavage of amide bond between TAF and TML, thereby releasing TAF or desphenyl TAF (C). Simultaneously, the phenol moiety on the phosphonamidate cleaves to give desphenyl analogs B and C, which were confirmed by ESI MS. Mass spectrometric data of the metabolites B and C were compared with calculated masses to confirm the chemical identity.

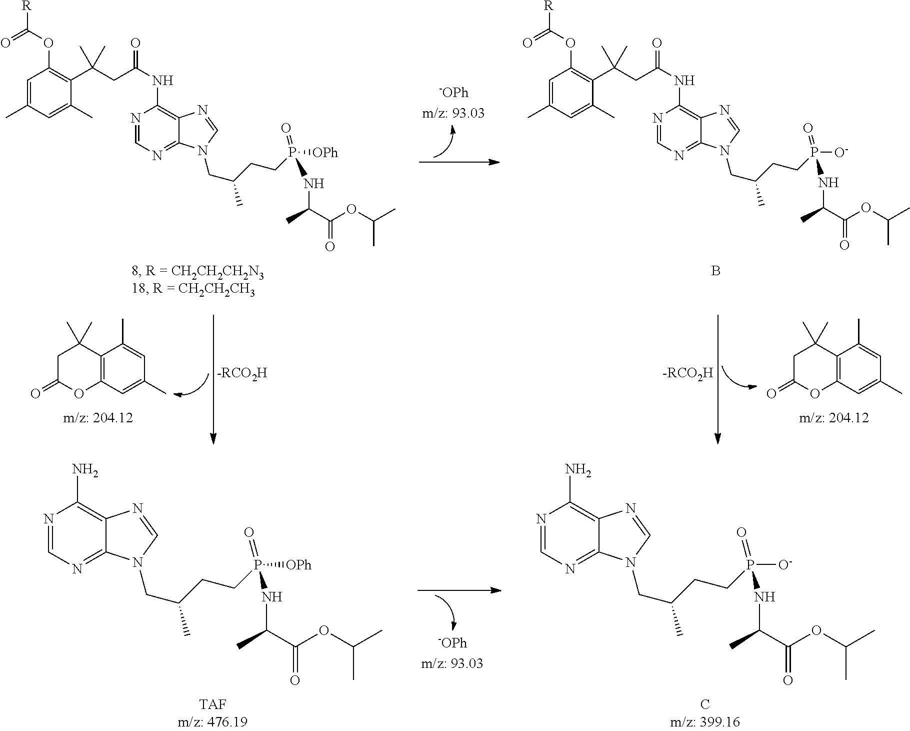

Further Examples Relating to Pop Materials Based on FTC and 3TC (Strategy 1):

(With reference to FIG. 3)

Procedures and Characterization for Synthesis of FTC POP Fragments

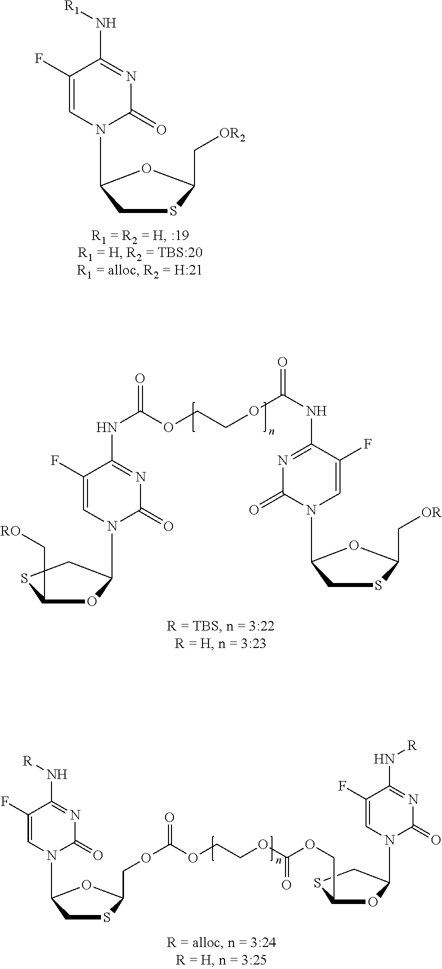

### Synthesis Schemes for N—N and O—O Linked FTC POP Fragments

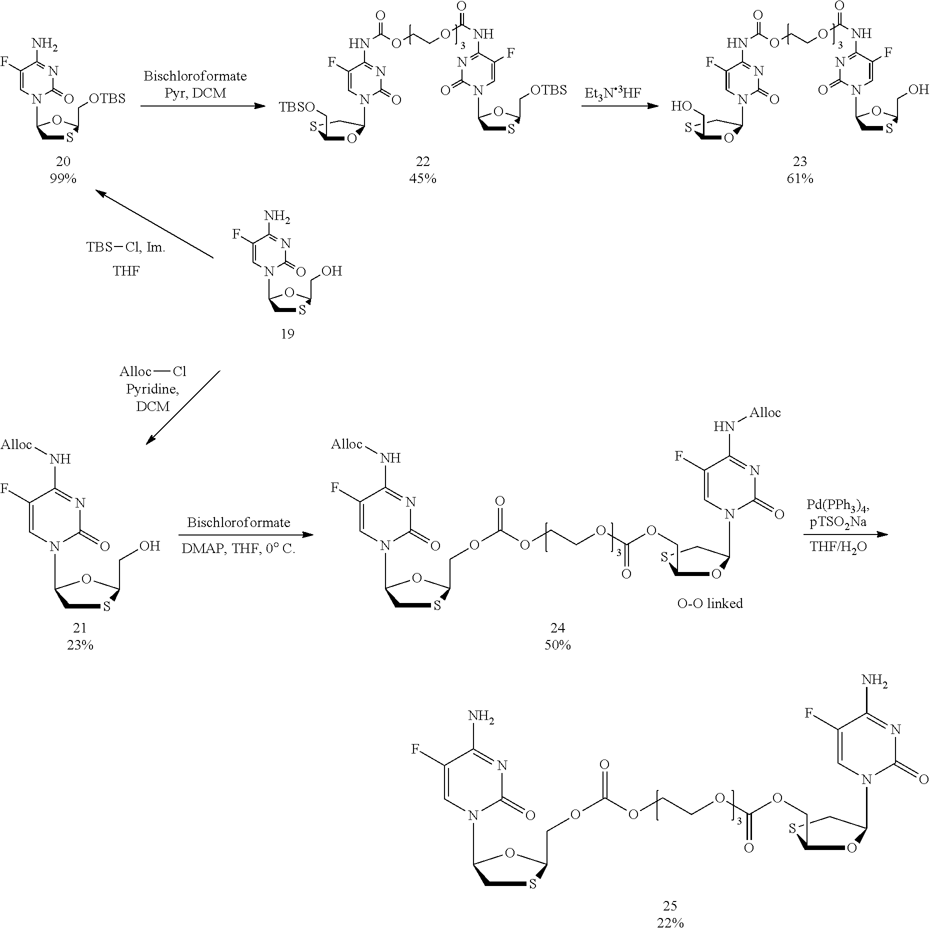

Synthesis of 4-amino-1-((2R,5S)-2-(((tert-butylmethylsilyl)oxy)methyl)-1,3-oxathiolan-5-yl)-5-fluoropyrimidin-2(1H)-one (20): In a flame-dried 100 mL round-bottom flask, cooled under argon, 19 (2 g, 8.09 mmol) was suspended in anhydrous tetrahydrofuran (27 mL). The reaction was initiated by addition of imidazole (881 mg, 12.9 mmol, 1.6 eq.) and tert-butyidimethylsilyl chloride (1.6 g, 10.5 mmol, 1.3 eq.). The resulting mixture was left to stir at ambient temperature. The reaction was deemed complete after 2 hours as monitored by TLC. The reaction was quenched with 2× volume of water and extracted 3× into ethyl acetate. The organic layer was washed with saturated sodium chloride solution, dried with MgSO₄, filtered, and condensed under reduced pressure. The resulting white/beige solid was used without further purification. 2.92 g, yield 99%; ¹H NMR (500 MHz, CDCl₃) δ ppm 0.16 (s, 6H) 0.95 (s, 9H) 3.23 (dd, J=12.5, 2.6 Hz, 1H) 3.54 (dd, J=12.5, 5.3 Hz, 1H) 3.96 (dd, J=11.9, 2.7 Hz, 1H) 4.23 (dd, J=11.9, 2.6 Hz, 1H) 5.24 (t, J=2.7 Hz, 1H) 6.30 (dt, J=4.8, 2.4 Hz, 1H) 8.32 (d, J=6.6 Hz, 1H).

Synthesis of (ethane-1,2-dlylbis(oxy))bis(ethane-2,1-diyl) bis((1-((2R,5S)-2-(((tert-butyldimethylsilyl)oxy)methyl)-1,3-oxathiolan-5-yl)-5-fluoro-2-oxo-1,2-dihydropyrimidin-4-yl)carbamate) (22): In a flame-dried 50 mL round-bottom flask, cooled under argon, 20 (200 mg, 0.55 mmol, 2.3 eq.) was suspended in 1 mL 0.76 M pyridine (0.843 mmol, 3.5 eq.) in distilled dichloromethane. The resulting mixture was cooled to 0° C. in an ice-water bath and initiated by the addition of tri(ethylene glycol) bis(chloroformate) (50 μL, 0.24 mmol, 1.0 eq.). The reaction was left to stir at 0° C., but was allowed to warm to ambient temperature. The reaction was deemed complete after 12 hours as monitored by TLC. The reaction was condensed under reduced pressure. The resulting residue was purified via silica flash chromatography (0-10% MeOH in DCM gradient). 100 mg, yield 45%; ¹H NMR (500 MHz, CDCl₃) δ ppm 0.14 (s, 12H) 0.93 (s, 18H) 3.24 (d, J=12.4 Hz, 2H) 3.52 (dd, J=12.3, 4.4 Hz, 2H) 3.66 (s, 5H) 3.76 (t, J4.5 Hz, 4H) 3.94 (dd, J=11.9, 1.4 Hz, 2H) 4.23 (d, J=11.8 Hz, 2H) 4.34 (br. s., 4H) 5.25 (br. s., 2H) 6.27 (br. s., 2H) 8.42 (br. s., 2H) 11.98 (br. s., 1H). HRMS (ESI) m/z: calc'd [M+H]⁺, 925.31; found 925.41.

Synthesis of (ethane-1,2-diylbis(oxy))bis(ethane-2,1-diyl) bis((5-fluoro-1-((2R,5S)-2-(hydroxymethyl)-1,3-oxathiolan-5-yl)-2-oxo-1,2-dihydropyrimidin-4-yl)carbamate) (23):³⁰⁶ In a 15 mL plastic conical tube, 22 (100 mg, 0.11 mmol) was dissolved in tetrahydrofuran and cooled to 0° C. in an ice-water bath. The reaction was initiated by the dropwise addition of triethylamine trihydrofluoride (175 μL, 1.07 mmol 10.0 eq.). The reaction was allowed to warm to ambient temperature with stirring and then left to stir overnight (16 hours). The reaction was condensed under reduced pressure and purified via C₁₈ flash chromatography (5-100% acetonitrile in water gradient) and lyophilized to yield a white solid. 46 mg, yield 61%; ¹H NMR (500 MHz, D₂O) δ ppm 3.27 (dd, J=12.7, 2.4 Hz, 2H) 3.62 (dd, J=12.7, 5.5 Hz, 2H) 3.68-3.75 (m, 4H) 3.76-3.84 (m, 4H) 3.93 (dd, J=13.0, 3.5 Hz, 2H) 4.09 (dd, J=13.0, 2.9 Hz, 2H) 4.26-4.39 (m, 4H) 5.35 (t, J=3.2 Hz, 2H) 6.20 (d, J=4.7 Hz, 2H) 8.50 (d, J=6.3 Hz, 2H). HRMS (ESI) m/z: calc'd [M+H]⁺, 697.14; found 697.34.

Synthesis of allyl (5-fluoro-1-((2R,5S)-2-(hydroxymethyl)-1,3-oxathiolan-5-yl)-2-oxo-1,2-dihydropyrimidin-4-yl)carbamate (21): In a flame-dried 50 mL round-bottom flask, cooled under argon, 19 (500 mg, 2 mmol) was suspended in distilled dichloromethane (3 mL). Pyridine (408 μL, 5 mmol, 2.5 eq.) was added to the flask, and the resulting mixture was cooled to 0° C. in an ice-water bath. The reaction was initiated by the dropwise addition of allyl chloroformate (500 μL, 4.7 mmol, 2.3 eq.). The reaction was stirred at 0° C. gradually warming to ambient temperature. The reaction was deemed complete after 15 hours as monitored by TLC. The reaction was condensed under reduced pressure, and the resulting residue was purified via silica flash chromatography (40-100% ethyl acetate in hexanes gradient) to yield a white solid. 156 mg, yield 23%; ¹H NMR (500 MHz, CDCl₃) δ ppm 2.53 (br. s., 1H) 3.24 (br. s., 1H) 3.54 (br. s., 1H) 3.97 (d, J=12.6 Hz, 1H) 4.19 (br. s., 1H) 4.70 (br. s., 3H) 5.21-5.47 (m, 4H) 5.98 (ddt, J=16.9, 11.0, 5.7, 5.7 Hz, 1H) 6.30 (d, J=3.8 Hz, 1H) 8.30 (br. s., 1H) 12.05 (br. s., 1H). HRMS (ESI) m/z: calc'd [M+H]⁺, 332.06; found 332.10.

Synthesis of diallyl (((2R,2′R,5S,5'S)-(3,14-dioxo-2,4,7,10,13,15-hexaoxahexadecane-1,16-diyl)bis(1,3-oxathiolane-2,5-diyl))bis(5-fluoro-2-oxo-1,2-dihydropyrimidine-1,4-diyl))dicarbamate (24): In a flame-dried 50 mL round-bottom flask, cooled under argon, 21 (100 mg, 0.3 mmol, 2.1 eq.) was suspended in anhydrous tetrahydrofuran. 4-Dimethylaminopyridine (53 mg, 0.4 mmol, 3.0 eq.) was added and the resulting mixture was cooled to 0° C. in an ice-water bath. The reaction was initiated by the addition of tri(ethylene glycol) bis(chloroformate) (30 μL, 0.14 mmol, 1.0 eq.). The reaction was stirred at 0° C. gradually warming to ambient temperature overnight. The reaction was deemed complete after 12 hours as monitored by TLC. The reaction was condensed under reduced pressure. The resulting residue was purified via silica flash chromatography (0-10% MeOH in DCM gradient) to obtain a semi-pure product (62 mg) that was used in the next reaction without further purification. HRMS (ESI) m/z: calc'd [M+H]⁺, 865.18; found 865.30.

Synthesis of bis(((2R,5S)-5-(4-amino-5-fluoro-2-oxopyrimidin-1(2H)-yl)-1,3-oxathiolan-2-yl)methyl) ((ethane-1,2-diylbis(oxy))bis(ethane-2,1-diyl)) bis(carbonate) (25): Compound 24 (62 mg, 0.07 mmol) was dissolved in anhydrous tetrahydrofuran (520 μL) under argon. The reaction was initiated by anaerobic addition of Tetrakis (triphenylphosphine) palladium (0) (6.5 mg, 0.006 mmol, 0.08 eq.), sodium p-toluenesulfinate (27 mg, 0.2 mmol, 2.2 eq.), and water (175 μL) followed by stirring at room temperature for 1.5 hours. The reaction was condensed under reduced pressure and purified via C₁₈ flash chromatography (5-100% acetonitrile in water gradient) and lyophilized. 11 mg, yield 22%; ¹H NMR (500 MHz, MeOD) δ ppm 3.21 (dd, J=12.1, 4.1 Hz, 2H) 3.55 (dd, J=12.3, 5.3 Hz, 2H) 3.63 (s, 4H) 3.73 (t, J=4.6 Hz, 4H) 4.31 (q, J=4.2 Hz, 4H) 4.54 (dd, J=12.4, 2.8 Hz, 2H) 4.60 (dd, J=12.3, 4.2 Hz, 2H) 5.45 (dd, 0.4.1, 2.8 Hz, 2H) 6.27 (t, J=3.8 Hz, 2H) 8.04 (d, J=6.8 Hz, 2H). HRMS (ESI) m/z: calc'd [M+H]⁺, 697.13; found 697.23.

### Synthesis of N—O Linked FTC POP Fragments

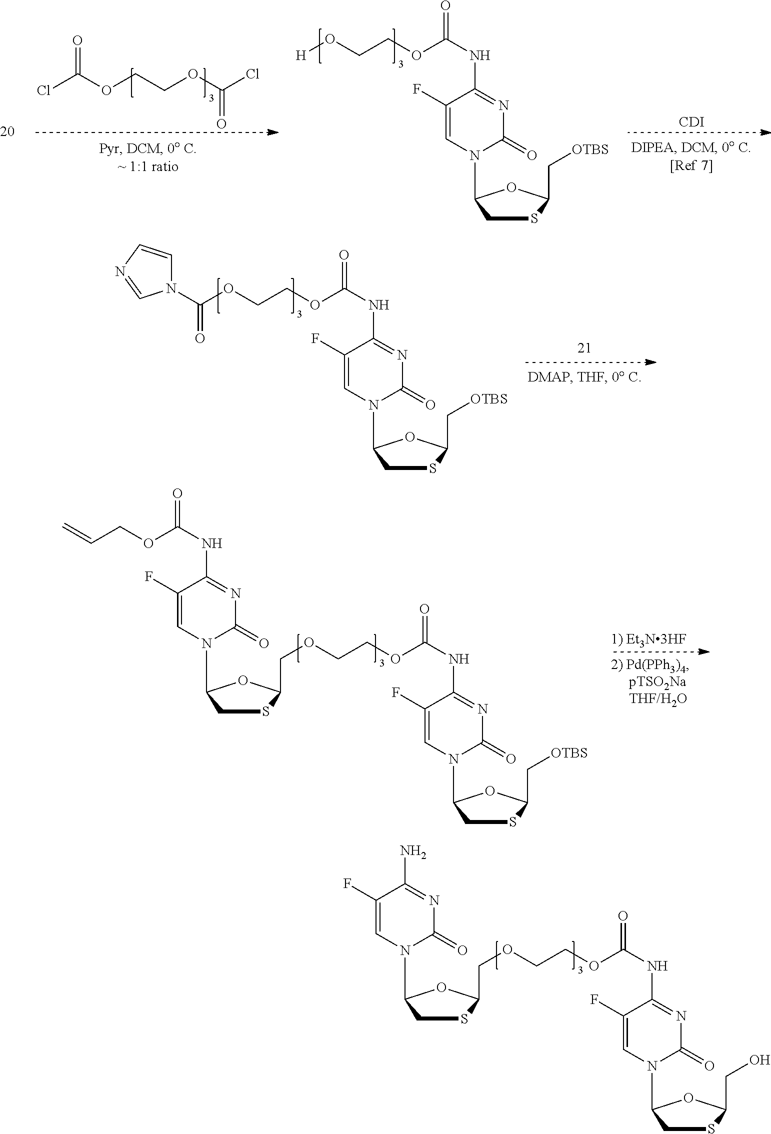

In Vitro Metabolism of FTC POP Fragments 23 and 25

General Procedure for in vitro metabolism studies of FTC dimers 23 and 25 via HPLC-UV: Reaction mixtures containing phosphate buffer (0.1 M, pH 7.4), mixed pooled gender human liver S9 (1-10.0 mg/mL final), mixed gender human skeletal muscle S9, or mixed gender human plasma are preincubated at 37° C. for 5 min. Reactions are initiated by the addition of FTC POP fragments (1 mM final, diluted from a 20 mM stock in DMSO). After incubation at 37° C., aliquots were taken at each time point and quenched in 2 volumes of Ice-cold methanol. The quenched aliquots were then centrifuged at 14000 rpm for 5 min. The supernatant was diluted 10-fold Into phosphate buffer (0.1 M, pH 7.4) and injected onto the HPLC for analysis (5% to 100% B over 8 minutes at a flow rate of 1 mL mi; solvent A: 50 mM Et₃NHOAc, pH 8; solvent B: acetonitrile) and read spectrophotometrically monitoring a decrease in substrate peak area at 305 nm (23) or 280 nm (25) over time. Quantification of 23 at each time point to determine rate was achieved by converting peak areas to nmols using a standard curve (23). Quantification of 25 at each time point to determine rate was achieved by calculating nmols based on the percent of total peak area at 250 nm (25+26, 200 nmol total). Hydrolysis of 25 yields two molar equivalents of 1, determined by quantifying 1 using a standard curve following full conversion of 25 to 1.

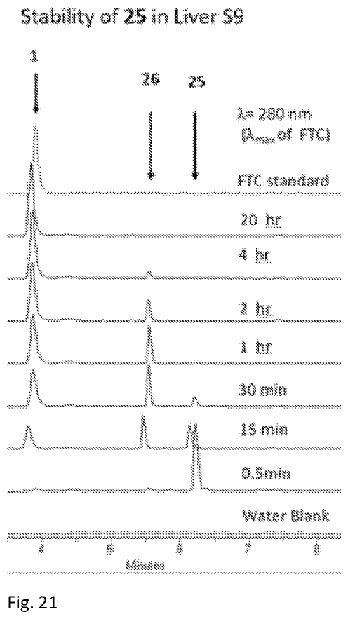

FIG. 21 shows representative HPLC traces for stability analysis of 25 (1 mM) in human liver S9 at λ=250 nm.

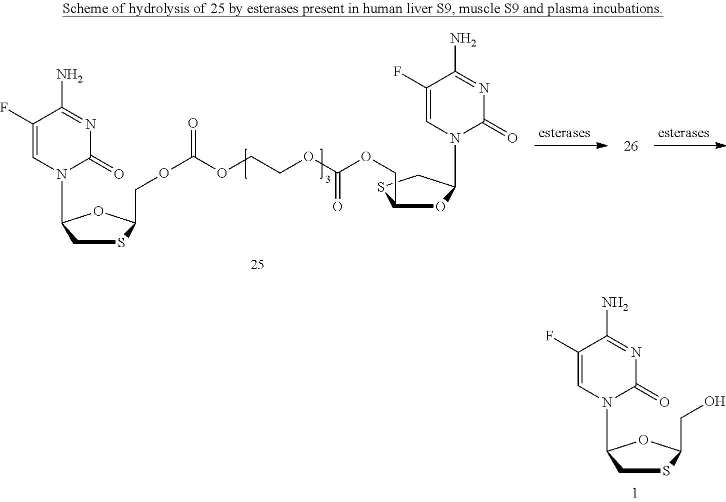

Rate of disappearance of starting material

| | Liver S9 | Muscle S9 | Plasma |
|---|---|---|---|
| | initial rate | initial rate | t_{1/2} |
| Compound | (nmol/h/mg) | (nmol/h/mg) | (h) |
| 23 | 0.6 ± 0.06 | N/A* | 13 ± 1.5 |
| | | *Non-linear trend | |
| 25 | 1014 ± 86 | 3.7 ± 0.4 | 0.45 ± 0.02 |

Summary table of stability of 23 and 25 in human liver S9, human skeletal muscle S9 and human plasma. In all cases, FTC formation was observed. In all conditions, the hydrolysis of 23 proceeded slower than 25. Neither 23 nor 25 hydrolyzed in phosphate buffer (0.1M, pH 7.4) after 48 hours (data not shown).

Human Tissues Used in In Vitro Metabolism of TAF-TML and FTC Dimers

All tissues were stored as directed (20° C. for human plasma and −80° C. for human liver S9 and muscle S9). Upon arrival, tissues were aliquoted into volumes needed for each assay to minimize freeze thaw.

Human Liver S9 Fraction-Pool of 50 (Sekuisi Xenotech, Product #HO610.59; 20 mg/mL stock)

Human S9-Skeletal Muscle (Bioreclamation IVT). Solution used for in vitro metabolism is a 50/50 mix of Male Human S9 (Bioreclamation IVT, Cat #S03517) and Female Human S9 (Bioreclamation IVT, Cat #S03518). Protein concentrations (mg/mL) were determined by averaging the concentrations provided by Bioreclamation.

Human Plasma (Bioreclamation IVT). Solution used for in vitro metabolism is a 50/50 mix of Human Plasma (Male) (Bioreclamation IVT, Cat #HMPLEDTA2-M) and Human Plasma (Female) (Bioreclamation IVT, Cat #HMPLEDTA2-F)

Further Work Relating to aryl carbamate TAF A₂ Monomers—Strategy 3

Conjugates of tenofovir alafenamide (TAF) featuring N⁶-alkyl amides and alkyl carbamates exhibit slow release of TAF. The rate of formation of desphenyl analogs surpasses N⁶ amide and carbamate cleavage. This technology describes studies toward the design and development of N⁶ aromatic carbamates of TAF with enhanced, tunable release of TAF. With variations in aromatic substitutions, the electronic and steric properties of aromatic carbamates can be altered to tune the release of TAF from its N⁶ conjugates. The present invention provides an approach in which aromatic carbamates are substituted with functional groups that allow polymerization via strategy 3.

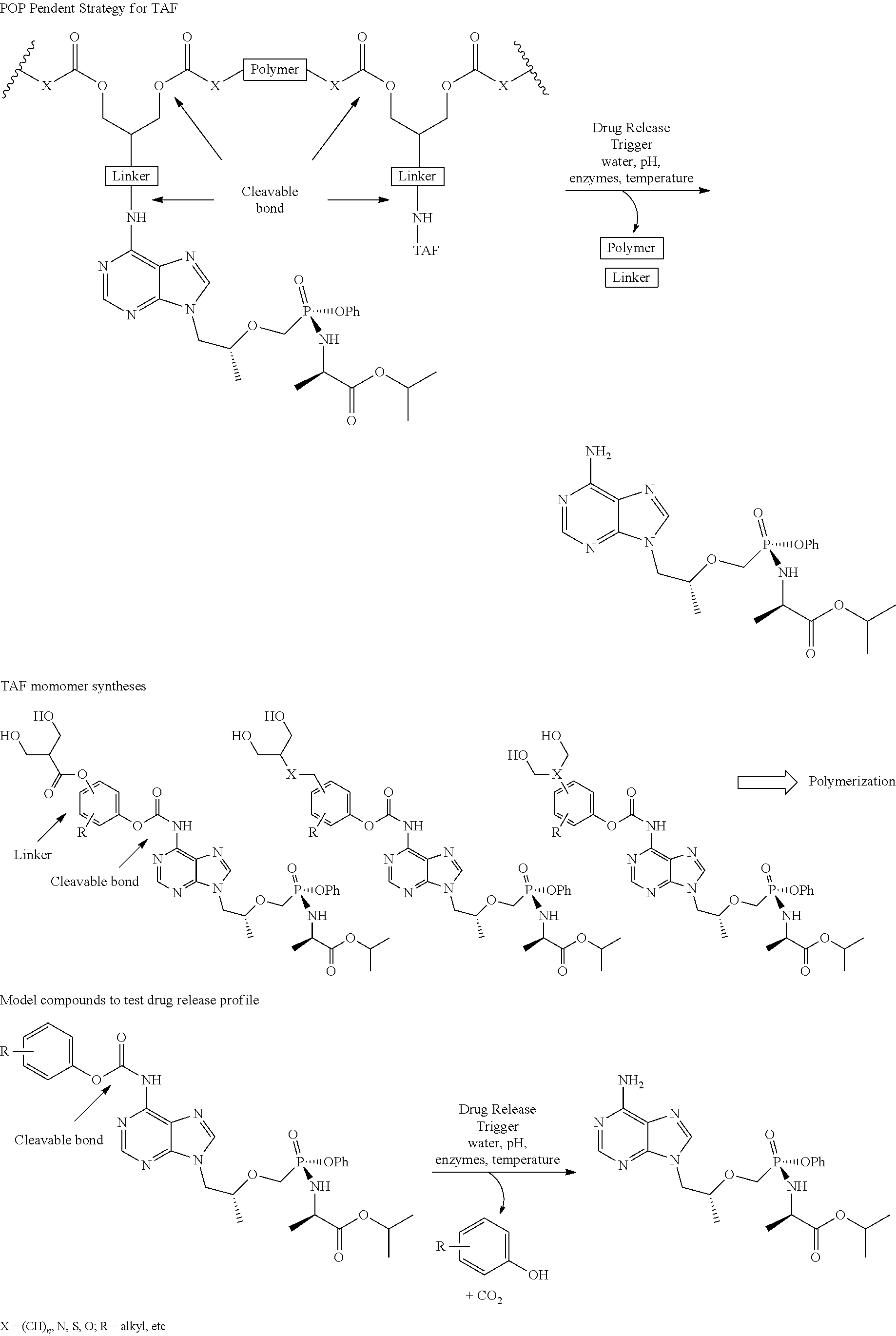

Model chemistry: A set of experiments have focused on synthesis of simple phenyl carbamates to study trends in activation kinetics toward design and synthesis of A₂ monomers for POP synthesis.

Synthesis of TAF carbamates: TAF phenyl carbamates can be synthesized by reacting either chloroformates, tetrazole carbamates or imidazium carbamates with TAF.

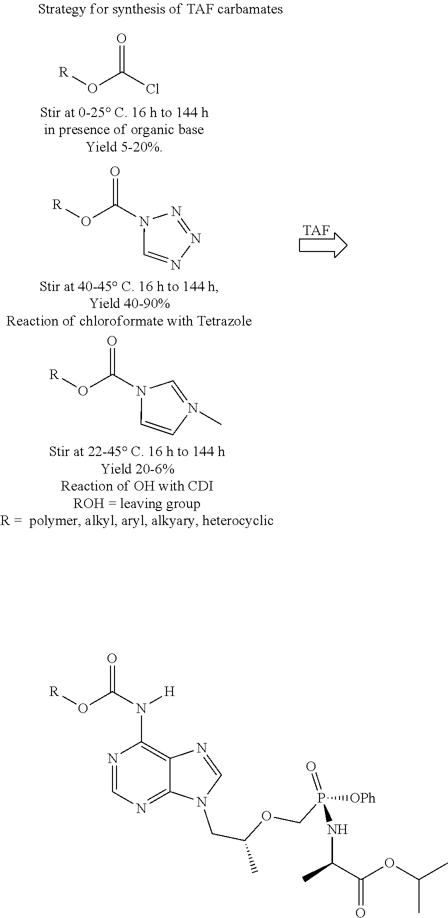

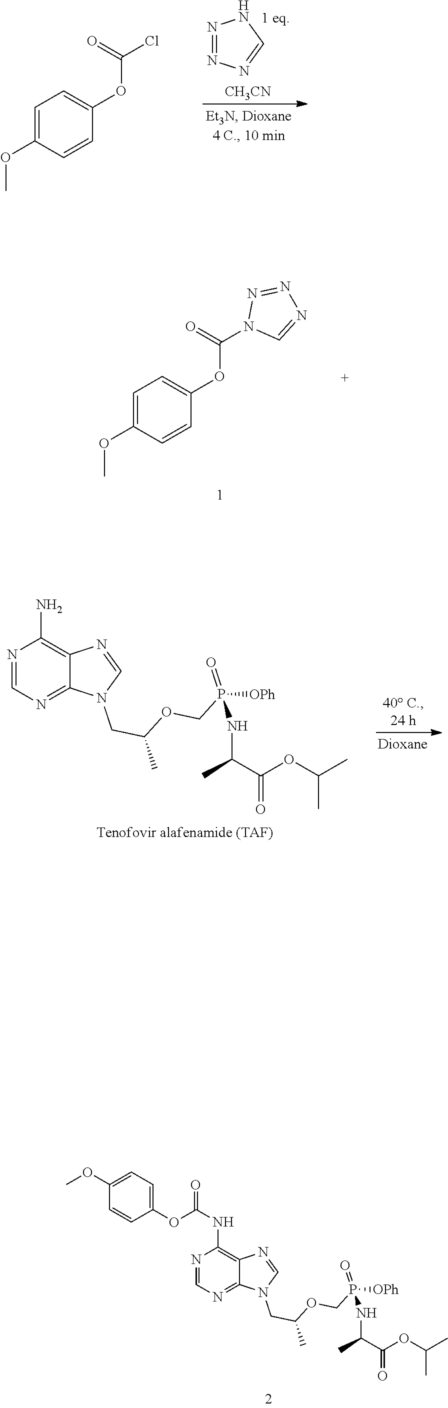

Synthesis of TAF carbamates using tetrazole Intermediates—Adapted from Tetrahedron Letters, 1977, 22, 1935-1936; Helvetica Chemica Acta, 1994, 77, 1267-1280.

General procedure for synthesis of tetrazole Intermediates (1). In a dry round-bottom flask 1H-Tetrazole (1 eq.) was stirred with triethylamine (1 eq.) in acetonitrile and dioxane at ambient temperature under argon atmosphere for 10-120 minutes. The reaction mixture was cooled in an ice-bath (0-4° C.) under argon atmosphere followed by dropwise addition of alkyl or aryl chloroformate dissolved in dioxane or as is. The reaction mixture was stirred at 0-10° C. for 10-120 minutes, then filtered and the tetrazole intermediate purified from the filtrate via crystallization or silica flash chromatography (ethyl acetate in hexanes). The product was obtained as amorphous solid or a viscous liquid.

General procedure for synthesis TAF carbamate using tetrazole Intermediates (2). In a dry round-bottom flask tetrazole intermediate (3-4 eq.) was stirred with tenofovir alafenamide (1 eq.) in dioxane at ambient temperature under argon atmosphere for 16-144 hours. The reaction mixture was concentrated in vacuo and the product purified via silica flash chromatography (0-100% ethyl acetate in hexanes, 0-10% methanol in methylene chloride). The product was obtained as amorphous solid or viscous liquid.

Synthesis of 1H-Tetrazole-1-carboxylic acid, 4-methoxyphenyl ester (1): In a dry round-bottom flask 0.45 M 1H-Tetrazole solution in acetonitrile (14.6 mL, 6.56 mmol) was stirred with triethylamine (0.92 mL, 6.56 mmol) in dioxane (20 mL) at ambient temperature under argon atmosphere for 10 minutes. The reaction mixture was cooled in an ice-bath (0-4° C.) under argon atmosphere followed by dropwise addition of 4-methoxyphenyl chloroformate. The reaction mixture was stirred at 0-10° C. for 10 minutes and the suspension filtered. Volatiles from the filtrate were removed in vacuo, and the residue dissolved in boiling methylene chloride (15 mL). The solution was cooled to ambient temperature and hexanes (10 mL) was added dropwise to obtain a suspension, which was filtered to obtain product as white crystalline powder (0.9 g, 62%). ¹H NMR (500 MHz, CDCl₃) δ ppm 9.33 (s, 1H), 7.29-7.27 (d, 2H), 7.01-6.99 (d, 2H, J=10 Hz), 3.86 (s, 3H).

Synthesis of N-(4-methoxyphenyl)-tenofovir alafenamide carbamate (2): In a dry round-bottom flask tetrazole intermediate 1 (100 mg, 0.45 mmol) was stirred with tenofovir alafenamide (54.15 mg, 0.113 mmol) in dioxane (10 mL) at 40° C. under argon atmosphere for 16 hours. Dioxane was removed in vacuo and the residue purified via silica flash chromatography (0-100% ethyl acetate in hexanes, 0-10% methanol in methylene chloride) to obtain 2 as a viscous liquid (47 mg, 66%). ¹H NMR (500 MHz, CDCl₃) δ ppm 8.79 (s, 1H), 8.60 (br s, 1H), 8.2 (s, 1H), 7.26-7.23 (t, 2H, J=7.5 Hz), 7.19-7.17 (d, 2H, J=10 Hz), 7.14-7.11 (t, 2H, J=7.5 Hz), 7.02-7.00 (d, 2H, J=10 Hz), 6.92-6.90 (d, 2H, J=10 Hz), 5.05-4.97 (m, 1H), 4.45-4.42 (d, 1H, J=15 Hz), 4.23-4.18 (dd, 2H, J=15 Hz, J=10 Hz), 4.02-3.97 (m, 2H), 3.95-3.91 (dd, 2H, J=15 Hz, J=10 Hz), 3.82 (s, 3H), 3.71-3.66 (dd, 1H, J=15 Hz, 1=10 Hz), 3.62-3.58 (t, 1H, J=10 Hz), 1.78 (s, 1H), 1.31-1.30 (d, 3H, J=5 Hz), 1.26-1.23 (m, 9H). ³¹P NMR (500 MHz, CDCl₃) δ ppm 20.63.

Synthesis of TAF carbamates using imidazolium Intermediates—Adapted from Tetrahedron Letters, 2004, 45, 3849-3853; Nature Protocols, 2008, 3(4), 646-654.

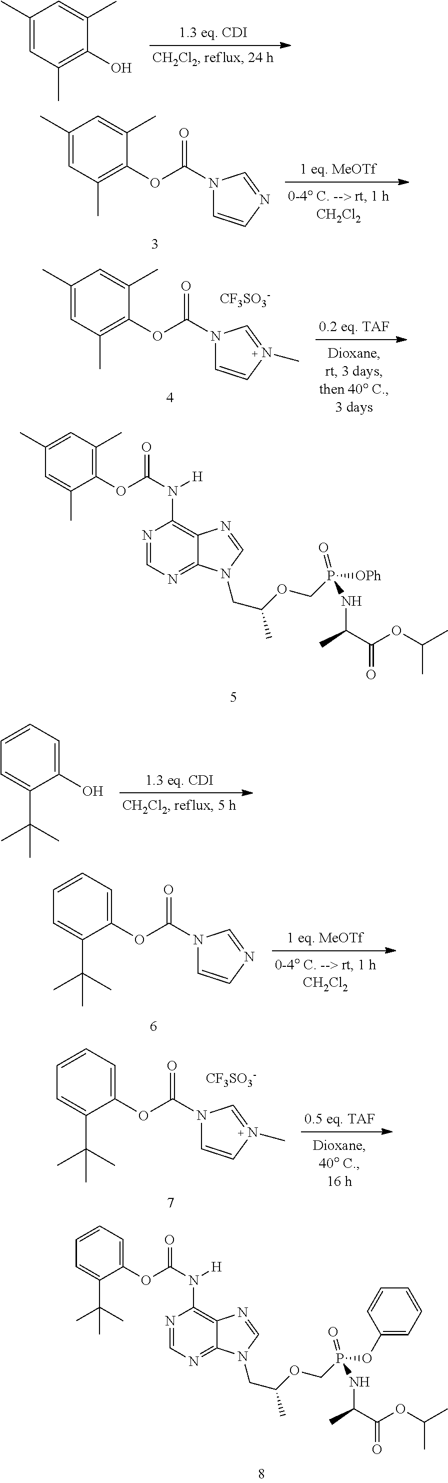

General procedure for synthesis of Imidazole Intermediates (3, 6). In a dry round-bottom flask 1,1′-carbonyldiimidazole (CDI, 1.3 eq.) was stirred with phenol or alcohol (ROH 1 eq.) In methylene chloride at 22-42° C. under argon atmosphere for 4-24 hours. The reaction mixture was concentrated in vacuo and the product purified via silica flash chromatography (0-100% ethyl acetate in hexanes). The product was obtained as amorphous solid or viscous liquid.

General procedure for synthesis of imidazolium Intermediates (4, 7). In a dry round-bottom flask imidazole intermediate (1 eq.) was dissolved in methylene chloride at ambient temperature under argon atmosphere. The solution was cooled on an ice-bath (0-4° C.) followed by dropwise addition of methyltrifluoromethanesulfonate or methyl halide. The reaction mixture stirred at 0-4° C. under argon atmosphere for 10-30 min and then warmed to ambient temperature. The product was isolated via crystallization using ether.

General procedure for synthesis TAF carbamates using imidazolium Intermediates (5,8). In a dry round-bottom flask imidazolium intermediate (1.5-4.5 eq.) was stirred with tenofovir alafenamide (1 eq.) in dioxane at ambient temperature under argon atmosphere for 16-144 hours. The reaction mixture was concentrated in vacuo and the product purified via silica flash chromatography (0-100% ethyl acetate in hexanes, 0-10% methanol in methylene chloride). The product was obtained as amorphous solid or viscous liquid.

Synthesis of 1H-Imidazole-1-carboxylic acid, 2,4,6-trimethylphenyl ester (3). In a dry round-bottom flask 1,1′-carbonyldiimidazole (778.6 mg, 4.77 mmol) was stirred with 2,4,6-trimethyl phenol (500 mg, 3.67 mmoL) in methylene chloride (20 mL) at 40-42° C. under argon atmosphere for 24 hours. The reaction mixture was concentrated in vacuo and the product purified via silica flash chromatography (0-50% ethyl acetate in hexanes). The product was obtained as white solid (802 mg, 95%). ¹H NMR (500 MHz, CDCl₃) δ ppm 8.34 (s, 1H), 7.61-7.60 (t, 1H, J=2.5 Hz), 7.18 (m, 1H), 6.94 (s, 2H), 2.31 (s, 3H), 2.19 (s, 6H).

Synthesis of imidazolium Intermediates (4): In a dry round-bottom flask imidazole intermediate 3 (500 mg, 2.173 mmol) was dissolved in methylene chloride (10 mL) at ambient temperature under argon atmosphere. The solution was cooled an ice-bath (0-4° C.) followed by dropwise addition of methyltrifluoromethanesulfonate (246 μL, 2.173 mmol). The reaction mixture stirred at 0-4° C. for 20 min, and then warmed to ambient temperature. Diethyl ether (1.5 mL) was added to the reaction mixture to obtain a suspension. Product was filtered from the suspension as white solid (560 mg, 65%). ¹H NMR (500 MHz, CDCl₃) δ ppm 9.84 (s, 1H), 7.88-7.87 (t, 1H, J=2.5 Hz), 7.55-7.54 (t, 1H, J=2.5 Hz), 6.93 (s, 2H), 4.22 (s, 3H), 2.31 (s, 3H), 2.2 (s, 6H).

Synthesis N-(2,4,6-trimethylphenyl)-tenofovir alafenamide carbamate (5). In a dry round-bottom flask imidazolium intermediate 4 (82.75 mg, 0.21 mmol) was stirred with tenofovir alafenamide (50 mg, 0.105 mmol) in dioxane (10 mL) at ambient temperature under argon atmosphere for 15 min then at 40° C. for 16 hours. The solvent were removed in vacuo and the residue purified via silica flash chromatography (0-100% ethyl acetate in hexanes, 0-10% methanol in methylene chloride) to obtain product as a viscous liquid (62.26 mg, 39%). ¹H NMR (500 MHz, DMSO-d₆) δ ppm 11.11 (s, 1H), 8.65 (s, 1H), 8.46 (s, 1H), 7.29-7.26 (t, 2H, 1=7.5 Hz), 7.13-7.10 (t, 1H, 0.1=7.5 Hz), 7.04-7.03 (d, 2H, J=5 Hz), 6.93 (s, 2H), 5.64-5.60 (t, 1H, 0.1=10 Hz), 4.87-4.80 (m, 1H), 4.43-4.40 (d, 1H, J=15 Hz), 4.29-4.25 (dd, 1H, J=15 Hz, 0.1=10 Hz), 4.0 (br s, 1H), 3.91-3.76 (m, 3H), 2.24 (s, 3H), 2.16 (s, 6H), 1.15-1.11 (m, 12H). ³¹P NMR (500 MHz, DMSO-de) 6 ppm 22.08.

Synthesis of 1H-Imidazole-1-carboxylic acid, 2-(1,1-dimethylethyl)phenyl ester (6). In a dry round-bottom flask 1,1′-carbonyldiimidazole (422 mg, 2.6 mmol) was stirred with 2-butyl phenol (300.4 mg, 2 mmoL) in methylene chloride (20 mL) at 40-42° C. under argon atmosphere for 5 hours. The reaction mixture was concentrated in vacuo and the product purified via silica flash chromatography (0-50% ethyl acetate in hexanes). The product was obtained as white solid (452.4 mg, 93%). 1H NMR (500 MHz, CDCl₃) δ ppm 8.36 (s, 1H), 7.62 (br s, 1H), 7.42 (br s, 1H), 7.3 (m, 2H), 7.21 (s, 1H), 7.16 (s, 1H), 1.39 (s, 9H).

Synthesis of imidazolium Intermediates 7: In a dry round-bottom flask imidazole intermediate 6 (250 mg, 1.023 mmol) was dissolved in methylene chloride (10 mL) at ambient temperature under argon atmosphere. The solution was cooled an ice-bath (0-4° C.) followed by dropwise addition of methyltrifluoromethanesulfonate (115.8 μL, 1.023 mmol). The reaction mixture stirred at 0-4° C. for 20 min and then warmed to ambient temperature. Diethyl ether (2 mL) was added to the reaction mixture to obtain a suspension. Product was filtered from the suspension as white solid (347 mg, 83%). ¹H NMR (500 MHz, CDCl₃) δ ppm 9.71 (s, 1H), 7.89 (br s, 1H), 7.57 (br s, 1H), 7.48 (br s, 1H), 7.32 (br s, 2H), 4.21 (s, 3H), 1.37 (s, 9H).

Synthesis N-(2-(1,1-dimethylethyl)phenyl)-tenofovir alafenamide carbamate (8). In a dry round-bottom flask imidazolium intermediate 7 (85.67 mg, 0.21 mmol) was stirred with tenofovir alafenamide (50 mg, 0.105 mmol) in dioxane (10 mL) at ambient temperature under argon atmosphere for 15 min then at 41° C. for 16 hours. The reaction mixture was concentrated in vacuo and the residue purified via silica flash chromatography (0-100% ethyl acetate in hexanes, 0-10% methanol in methylene chloride) to obtain product as a viscous liquid. 1H NMR (500 MHz, DMSO-de) δ ppm 11.26 (s, 1H), 8.67 (s, 1H), 8.47 (s, 1H), 7.41-7.39 (d, 1H, J=10 Hz) 7.30-7.27 (t, 3H, 0.1=7.5 Hz), 7.22-7.19 (t, 1H, J=7.5 Hz), 7.14-7.13 (d, 2H, 0.1=5 Hz), 7.05-7.04 (d, 2H, 0.1=5 Hz), 5.66-5.61 (t, 1H, 0.1=12.5 Hz), 4.88-4.81 (m, 1H), 4.45-4.42 (d, 1H, 0.1=15 Hz), 4.31-3.26 (dd, 1H, J=15 Hz, J=10 Hz), 4.02 (br s, 1H), 3.92-3.78 (m, 3H), 1.35 (s, 9H), 1.16-1.15 (d, 6H, J=5 Hz), 1.13-1.1 (d, 6H, J=10 Hz). ³¹P NMR (500 MHz, DMSO-de) δ ppm 22.08.

Hydrolysis of TAF-aryl carbamate conjugates: Prodrug hydrolysis experiments assess the relative stability of compounds 2, 5 and 8. To evaluate the hydrolysis of TAF aromatic carbamates, HPLC coupled with UV detection is used with the following method: 40% B for 1 min, 40% to 100% B from 1 to 16 min, 100% B from 16 to 18 min at a flow rate of 1 mL min⁻¹ (solvent A: NH₄OAc (50 mM, pH 6), solvent B: methanol). Phosphate Buffer (100 mM, pH 7.4) was Preincubated at 37° C. for 5 Minutes. TAF aromatic carbamates are added and the reaction is incubated at 37° C. Aliquots are taken at each time point and injected onto the HPLC for analysis. The HPLC profiles are visualized at 269 nm or 270 nm. Chromatographic evidence indicates hydrolytic cleavage takes place fastest for compound 2 and other compounds.

Further Work Relating to carbamate 5

Carbamate 5 was synthesized according to the process described above. Purity. The compound was dissolved in DMSO (100 μM) and injected on HPLC to determine purity of 95.1% at 269-270 nm. Standard Curve. Serial dilution of carbamate 5 in DMSO (100 μM to 0.78 μM) was done and each sample injected on HPLC. The UV absorbance at different concentrations was used to obtain a linear standard curve. The standard curve was used in determining solubility in buffers, kinetics of degradation at pH 7.4 and stability under polymerization conditions. Solubility. Carbamate 5 in DMSO was mixed with 100 mM phosphate buffer pH7.4 with varying amounts of DMSO (0.1% v/v, 0.5% v/v, 1% v/v and 5% v/v). The mixtures were centrifuged at 14000 rpm for 5 min and the supernatant injected on HPLC. The UV absorbance of carbamate 5 was compared with linear standard curve to determine the amount injected and correlate the concentration and solubility in 100 mM phosphate buffer pH 7.4. A concentration of 111.2 μM with 5% DMSO v/v can be achieved in 100 mM phosphate buffer pH 7.4.

Kinetics of TAF release from carbamate 5 at pH 7.4. A 40 μM solution of carbamate 5 in 100 mM phosphate buffer with 5% v/v was incubated at 37° C. Aliquot of 250 μL was injected on HPLC at regular intervals to determine the amount of carbamate 5 left and amount of tenofovir alafenamide (TAF) formed using a standard curve. In 150 minutes significant amount of carbamate 5 is converted to TAF, at which point the analysis was stopped. The nanomoles of carbamate 5 and TAF were plotted against time to obtain curves and illustrate the profile of each compound. A log plot of carbamate 5 vs time gave a straight line, indicating a first order kinetics of metabolism at pH 7.4. This plot was used to determine a half-life of 53.7 min for 40 μM carbamate 5 in 100 mM phosphate buffer pH7.4 at 37° C.

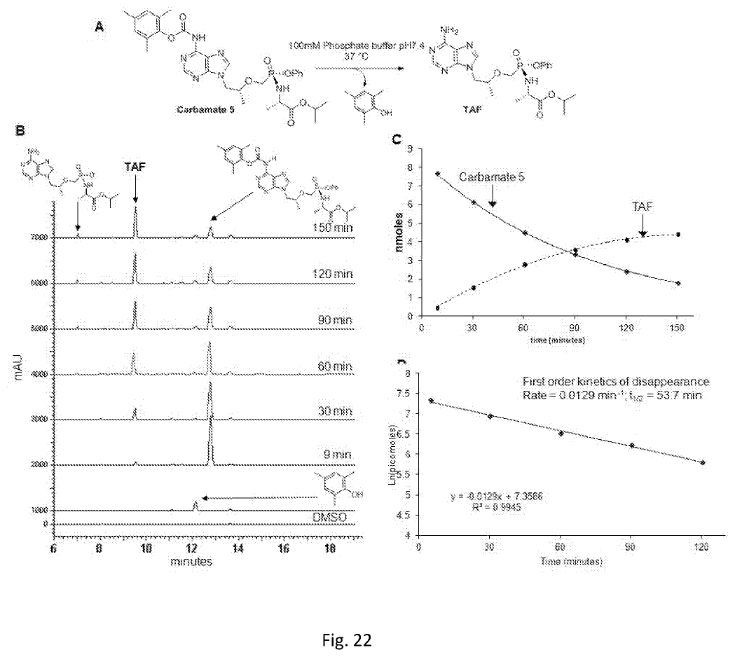

FIG. 22 shows the kinetics of TAF release from carbamate 5 in Phosphate Buffer pH 7.4: A. Representation of chemical transformation in carbamate 5 leading to release of tenofovir alafenamide (TAF); B. Chromatographic evidence of time dependent degradation of carbamate 5 and release of TAF; C. Graphical representation of TAF release from carbamate 5; and D. Log plot of carbamate 5 degradation and calculation of half-life at pH7.4 at 37° C.

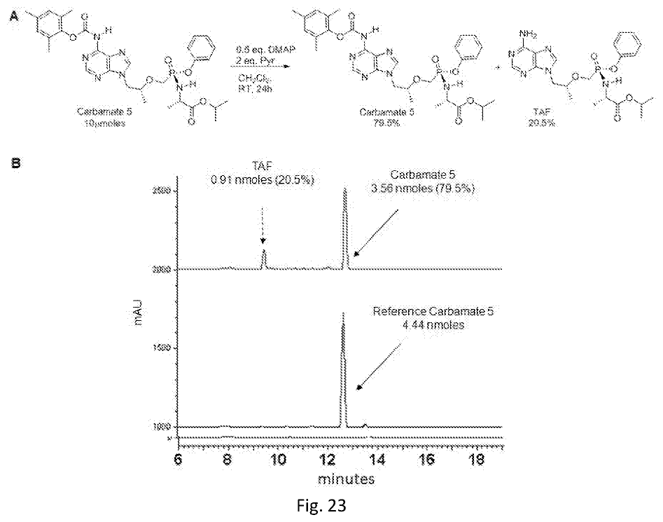

Stability of compound 5 under polymerization conditions. Carbamate 5 is shown to release TAF in a time bond manner. To determine the stability of carbamate moiety under polymerization conditions and evaluate the utility of TAF phenyl carbamates, carbamate 5 was tested for its stability under model polymerization conditions. A solution of carbamate 5 in methylene chloride (CH₂Cl₂) was stirred with 0.5 molar equivalent of 4-Dimethylaminopyridine and 2 molar equivalents of pyridine at room temperature (RT, 22-25° C.) for 24 h. The portion of the reaction mixture was removed and dissolved in DMSO followed by analysis of the reaction mixture using HPLC to determine amount of TAF formed and carbamate 5 left after 24 h. Standard curve was used to determine that 20.5% TAF was formed after 24 hours, leaving 79.5% carbamate 5 intact (FIG. 23).

FIG. 23 shows the stability of Carbamate 5 under polymerization conditions: A. Chemical transformation of carbamate 5 under polymerization conditions; B. Analysis of carbamate 5 mixture with DMAP/Pyridine after 24 h. A reference sample of carbamate 5 was used to determine the percentage of TAF and carbamate 5 left in the reaction mixture.

### Synthesis of Monomer for TAF Polymerization Based on carbamate 5

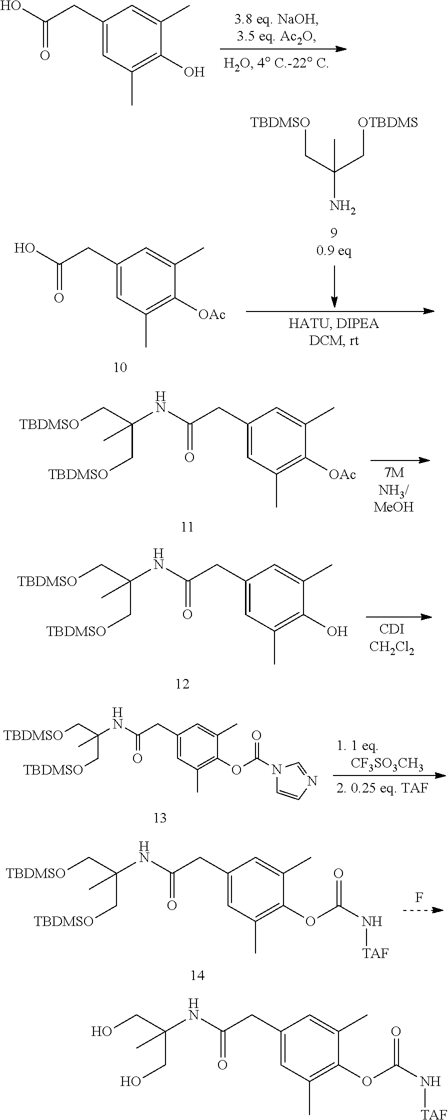

Synthesis of 2-amino-2-methyl-1,3-(bis-O-tert-butyldimethylsilyl)-propandiol (9): To a dry flask containing 2-amino-2-methyl-1,3-propandiol (210 mg, 2 mmol), imidazole (27.3 mg, 4 mmol), anhydrous CH₂Cl₂ (10 mL) was added and stirred under inert atmosphere for 2 h to get a homogenous mixture. To this mixture, a solution of tert-butyldimethylsilyl chloride (603 mg, 4 mmol) in anhydrous CH₂Cl₂ (10 mL) was added dropwise and the reaction mixture was stirred for 16-24 h at 22-25° C. to get a white suspension. The reaction mixture was washed with water (40 mL). To the organic phase, 7N NH₄OH (5 mL) was added followed by brine wash (40 mL). CH₂Cl₂ was removed in vacuo and the residue dried on high vacuum for 16 h to obtain product as a colorless syrup (441 mg, 66.17%). ¹H NMR (500 MHz, CDCl₃) δ ppm 3.375 (dd, 4H, J=15 Hz, J=10 Hz), 0.96 (s, 3H), 0.9 (s, 18H), 0.05 (s, 12H).

Synthesis of 4-(acetyloxy)-3,5-dimethyl phenylacetic acid (10): A solution of 3,5-dimethyl-4-hydroxy-phenylacetic acid (1.8 g, 10 mmol) in 2N NaOH (40 mL) was cooled on ice bath (4-10° C.) and acetic anhydride (3.31 mL, 35 mmol)) was added dropwise over 10 min. After stirring the reaction mixture on ice bath for 30 min, the ice bath was removed and reaction mixture was stirred for 16-24 h at 22-25° C. to get a white suspension. The suspension was acidified using 1N HCl and product extracted in EtOAc (200 mL). The organic layer was washed with water, brine and dried over MgSO₄. EtOAc was evaporated and the solid residue dried on high vacuum for 16 h to obtain product as off-white solid (2.04 g, 86.4%). ¹H NMR (500 MHz, CDCl₃) δ ppm 7.00 (s, 2H), 3.56 (s, 2H), 2.34 (s, 3H), 2.14 (s, 6H).

Synthesis of 11: To a suspension of 9 (358 mg, 1.07 mmol), 10 (281.7 mg, 1.19 mmol), HATU 498.3 mg, 1.31 mmol) in anhydrous CH₂Cl₂ (20 mL) under inert atmosphere, N,N-Diisopropylethylamine (415.3 μL, 2.38 mmol) was added dropwise to get a pale yellow solution. The reaction mixture was stirred for 16-24 h at 22-25° C. to get a yellow solution, which was purified by flash chromatography to obtain product as colorless syrup (519.7 mg, 90.04%). R_{f} 0.5 in 1:9 EtOAc:Hexanes. ¹H NMR (500 MHz, CDCl₃) δ ppm 6.94 (s, 2H), 5.77 (br s, 1H), 3.73 (d, 2H, J=10 Hz), 3.42 (d, 2H, =101 Hz), 3.4 (s, 2H), 2.34 (s, 3H), 2.13 (s, 6H), 1.3 (s, 3H), 0.84 (s, 18H), 0.005 (d, 12H, J=2.5 Hz).

Synthesis of 12: A solution of 11 (491.8 mg) in 7N NH₄OH (10 mL) was stirred for 72 h at 22-25° C. to get a pink solution, which was purified by flash chromatography to obtain product as colorless syrup (400.7 mg, 88.38%). R, 0.5 in 1:9 EtOAc:Hexanes. ¹H NMR (500 MHz, CDCl₃) δ ppm 6.83 (s, 2H0, 5.78 (br s, 1H), 4.68 (s, 1H), 3.71 (d, 2H, J=10 Hz), 3.39 (d, 2H, J=10 Hz), 3.37 (s, 2H), 2.21 (s, 6H), 1.3 (s, 3H), 0.82 (s, 18H), 0.015 (d, 12H, J=2.5 Hz).

Synthesis of 13: To a refluxing solution of 12 (369.7 mg, 0.745 mmol, 41° C.) in anhydrous CH₂Cl₂ (10 mL) under inert atmosphere, 1,1′-Carbonyldiimidazole (628.6 mg, 3.87 mmol) was added in four lots, upon which the starting material was consumed. The reaction was purified by flash chromatography to obtain product as colorless syrup (439.83 mg, 88.66%). R_{f} 0.3 in 1:3 EtOAc:Hexanes. ¹H NMR (500 MHz, CDCl₃) δ ppm 8.32 (s, 1H), 7.59 (t, 1H, J=1.5 Hz), 7.19 (t, 1H, J=1 Hz), 7.02 (s, 2H), 5.78 (br s, 1H), 3.74 (d, 2H, J10 Hz), 3.45 (t, 4H, J=5 Hz), 2.21 (s, 6H), 1.32 (s, 3H), 0.86 (t, 18H, J=3 Hz), 0.02 (dd, 12H, J=5 Hz, J=2.5 Hz).

Synthesis of 14: To a cooled solution (0-4° C.) of 14 (58 mg, 98.3 μmol) in anhydrous CH₂Cl₂ (5 mL) under inert atmosphere, methyltrifluoromethanesulfonate or methyl halide (12.2 μL, 108.15 μmol) was added dropwise and the solution stirred at 0-4° C. for 30 min and then at 22-25° C. for 30 min. The organic solvent was removed in vacuo and the residue dried over high vac for 30 min. The residue was stirred with Tenofovir Alafenamide (11.7 mg, 24.57 μmol) in Dioxane (4 mL) under inert atmosphere for 16-24 h at 40° C. Dioxane was evaporated in vacuo and the residue purified by flash chromatography to afford compound 6 as a colorless syrup (3 mg, 14%). ¹H NMR (500 MHz, CDCl₃) δ ppm 8.79 (s, 1H), 8.56 (br s, 1H), 8.20 (s, 1H), 7.26-7.24 (d, 2H, J=10 Hz), 7.14-7.11 (t, 1H, J=7.5 Hz), 7.03-7.02 (d, 2H, J=5 Hz), 6.96 (s, 2H), 5.76 (br s, 1H), 5.05-4.97 (m, 1H), 4.46 (dd, 1H, J=14.3 Hz, 2.7 Hz), 4.22 (dd, 1H, J14.3, 7.7 Hz), 4.08-3.98 (m, 2H), 3.94 (dd, 1H, J=13.5 Hz, 7.5 Hz), 3.74 (d, 2H, J=10 Hz), 3.72-3.66 (m, 1H), 3.57 (t, 1H, J=10.4 Hz), 3.45, 3.43 (2s, 4H), 2.24 (s, 6H), 1.32-1.30 (m, 6H), 1.27-1.21 (m, 12H), 0.85 (s, 18H), 0.02-0.01 (d, 12H, J=5 Hz).

Thus the following and related structures are examples of useful compounds in the present context.

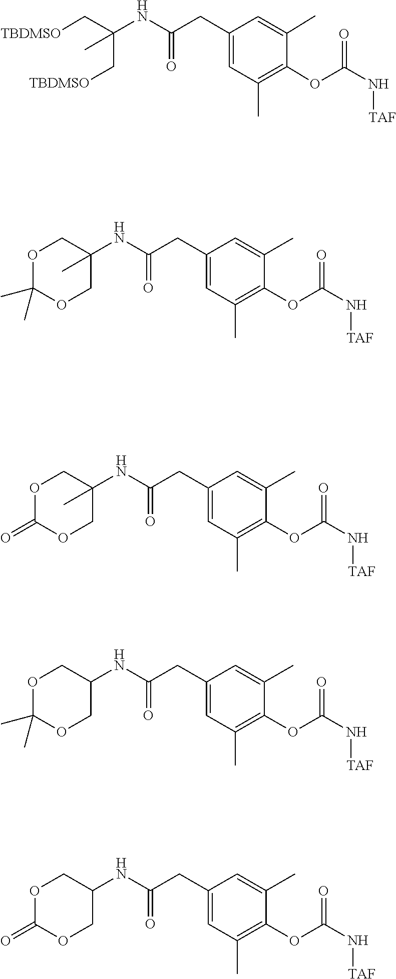

Further Work Relating to aryl and Branched alkyl carbamate A₂ Monomers Containing FTC

Alkyl and polyethylene glycol linkers within FTC POP fragments linked through the amine group (representing carbamate-containing fragments of polymers generated by strategy 1) have measured low release rates from FTC. To provide an approach to enhance and tune the kinetics of FTC release from carbamate-containing POP fragments, we have developed aromatic N-masking groups. When incorporated at the free amine as carbamates, these are designed to undergo unmasking of the FTC amine at higher rates. Further, this technology enables tuning of release rates through variation of the ring substituents (introduction of electron donating and withdrawing groups). Three applications of aryl masking groups in POP synthesis are applicable:

1) Novel linkers with tunable activation kinetics can be incorporated into FTC-containing A₂ monomers that can be used in POP synthesis by strategy 2 (polymerization through reaction of 5′-OH groups).

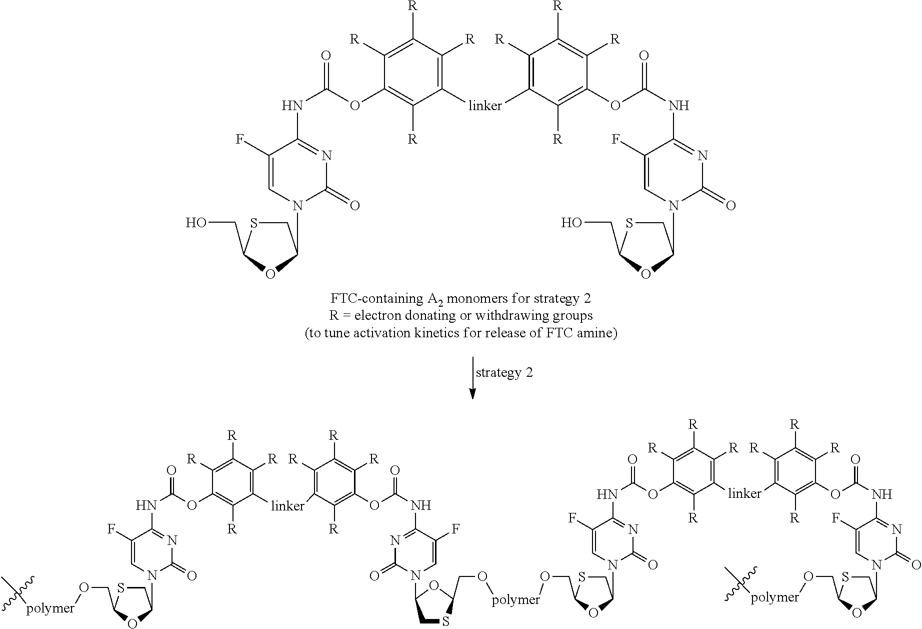

2) N,N′-Disubstituted FTC analogs bearing reactive hydroxyls used as A₂ monomers in strategy 3.

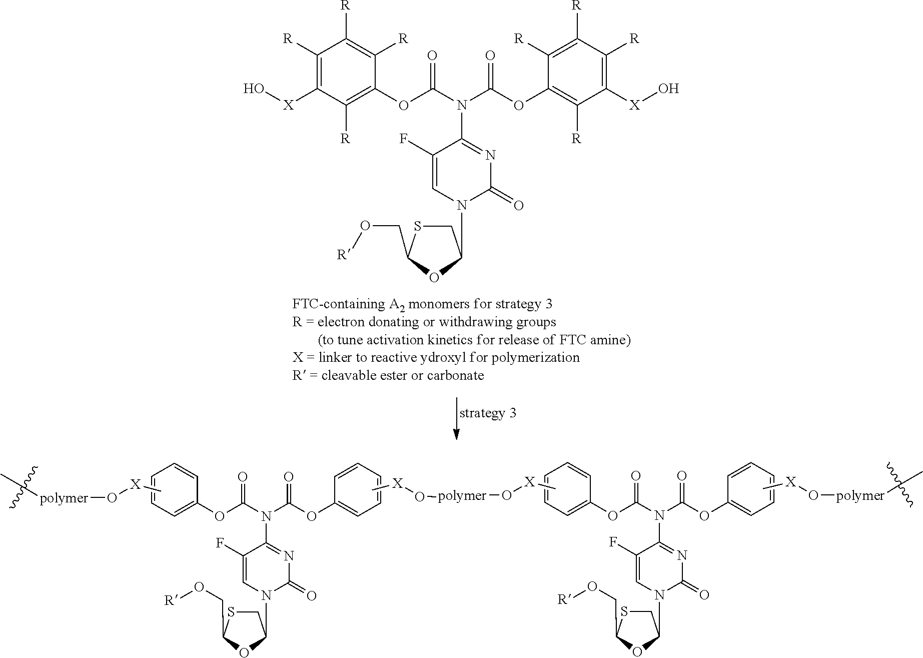

3) Aryl masking groups bearing a reactive diol incorporated to provide A₂ monomers for strategy 3.

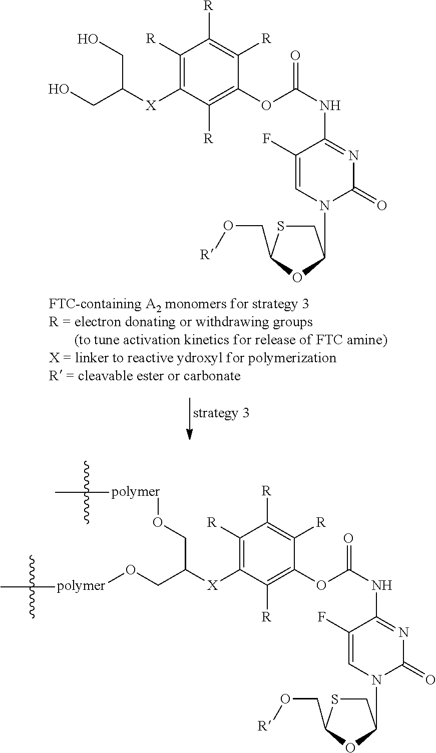

Model chemistry: We have focused on synthesis of simple phenyl carbamates and N,N′-disubstituted analogs to study trends in activation kinetics toward design and synthesis of A₂ monomers POP synthesis. Results indicate that FTC N-phenylcarbamate (5) is rapidly hydrolyzed and degrades during synthesis. In contrast, FTC N-p-methoxyphenylcarbonyl-N-p-methoxyphenyl carbamate (8) is more stable during purification, allowing for activation experiments, indicating that we can tune activation kinetics.

General Synthetic Scheme FTC N-arylcarbamates:

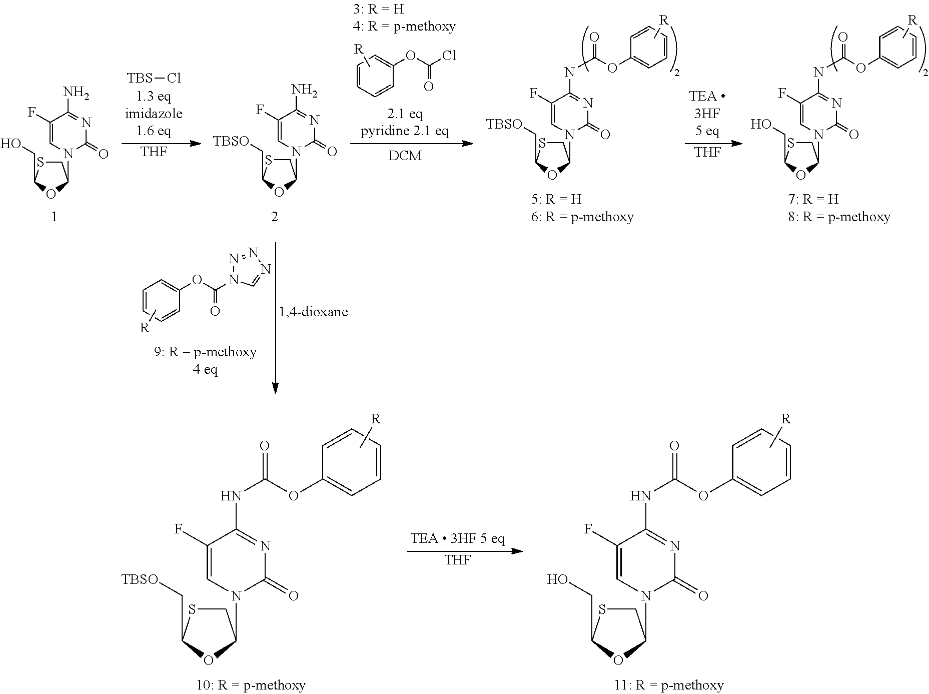

Synthesis of 4-amino-1-((2R,5S)-2-(((ter-butyldimethylsilyl)oxy)methy)-1,3-oxathiolan-5-yl)-5-fiuoropyrdmidin-2(1H)-one (2): In a dry round-bottom flask, 1 (1 eq, 8.1 mmol) was dissolved in anhydrous tetrahydrofuran (31 mL) under argon. Imidazole (1.6 eq, 12.9 mmol) was added to the solution and the flask was cooled in an ice/water bath. Following temperature equilibration in the ice bath, t-butyldimethylsilyl chloride (1.3 eq, 10.5 mmol) was added with stirring. The reaction was allowed to slowly warm to room temperature and stirred overnight under argon. The reaction was quenched with water (60 mL) and diluted with ethyl acetate (100 mL). The aqueous layer was extracted two times with 100 mL ethyl acetate. The organic layers were combined, dried over MgSO₄, filtered, and condensed under reduced pressure to yield a pale yellow solid. This product was used without further purification. (2: 95%, 7.68 mmol). 1H NMR (500 MHz, chloroform-d) δ_{H} ppm 8.29 (1H, d, J=6.60 Hz) 7.74 (1H, s) 7.12 (2H, s) 6.24-6.36 (1H, m) 5.24 (1H, t, J=2.67 Hz) 4.21 (1H, dd, J=11.95, 2.67 Hz) 3.95 (1H, dd, J=11.87, 2.59 Hz) 3.53 (1H, dd, J=12.50, 5.27 Hz) 3.21 (1H, dd, J=12.42, 2.67 Hz) 0.95 (9H, s) 0.15 (6H, s)

Synthesis of 4-N-p-methoxyphenylcarbonyl-N-p-methoxyphenylcarbamate-1-((2R,5S)-2-(((tert-butyldimethylsilyl)oxy)methyl)-1,3-oxathiolan-5-yl)-5-fluoropyrimidin-2(1H)-one (6): In a dry round-bottom flask, 2 (1 eq, 0.55 mmol) was dissolved in 1.1 mL anhydrous dichloromethane under argon. Pyridine (2.1 eq, 1.16 mmol) was added and the solution was cooled in an ice/water bath. Once the reaction solution has equilibrated to the ice/water bath, 4 (2.1 eq, 1.16 mmol) was added dropwise with stirring. The reaction was allowed to slowly warm to room temperature and stirred overnight under argon. The reaction was quenched with water (20 mL) and diluted with anhydrous dichloromethane (40 mL). The organic layer was washed two times with 20 mL water, then saturated ammonium chloride, and then brine. The organic layer was then dried over MgSO₄ and concentrated under reduced pressure. The product was purified by silica flash chromatography (ethyl acetate/hexanes mobile phase). Note: This synthesis was attempted for compound 5, but was unstable and degraded during purification. (6: 66%, 0.37 mmol). 6: 1H NMR (500 MHz, chloroform-d) δ_{H} ppm 9.02 (1H, d, J=5.19 Hz) 7.08-7.19 (4H, m) 6.82-6.98 (4H, m) 6.30 (1H, d, J=5.19 Hz) 5.31 (1H, t, J=1.96 Hz) 4.34 (1H, dd, J=12.34, 1.81 Hz) 3.98 (1H, dd, J=12.42, 1.89 Hz) 3.78 (6H, s) 3.66 (1H, dd, J=13.13, 5.27 Hz) 3.43 (1H, d, J=13.05 Hz) 0.95 (9H, s) 0.15 (6H, s)

Synthesis of 4-N-p-methoxyphenylcarbonyl-N-p-methoxyphenylcarbamate-1-((2R,5S)-2-(hydroxymethyl)-1,3-oxathiolan-5-yl)-5-fluoropyrimidin-2(1H)-one (8): In a 15 mL plastic conical tube, 6 (1 eq, 0.37 mmol) was dissolved in tetrahydrofuran (3.7 mL). Triethylamine trihydrofluoride (5 eq, 1.83 mmol) was added dropwise, open to air, with stirring. The reaction was loosely covered and stirred overnight at room temperature. The reaction was rapidly transferred to a carrot flask and concentrated under reduced pressure to yield a light yellow oil. This was then purified by reverse phase, C18 flash chromatography (water/acetonitrile mobile phase). 1H NMR (500 MHz, methanol-d4) δ_{H} ppm 9.43 (1H, br. s.) 7.14 (4H, br. s.) 6.97 (4H, br. s.) 5.40 (1H, br. s.) 3.90-4.23 (2H, m) 3.80 (6H, br. s.) 3.45-3.75 (1H, m) 3.16 (1H, br. s.)

Synthesis of 4-N-p-methoxyphenylcarbamate-1-((2R,5S)-2-(((tert-butyldimethylsilyl)oxy)methyl)-1,3-oxathiolan-5-yl)-5-fluoropyrimidin-2(1H)-one (10): In a round-bottom flask, 2 (1 eq, 0.14 mmol) and 9 (4 eq, 0.55 mmol) were combined at room temperature with 700 μL 1,4-dioxane. The reaction was lightly capped, heated to 40° C., and stirred overnight. The reaction was then concentrated under reduced pressure and purified via silica flash chromatography (ethyl acetate/hexanes mobile phase) (10: 41%, 0.06 mmol). 1H NMR (500 MHz, chloroform-d) δ_{H} ppm 8.78-8.89 (1H, m) 7.16-7.23 (2H, m) 6.89-6.95 (2H, m) 5.24-5.33 (0H, m) 3.93-4.32 (0H, m) 3.84-3.88 (2H, m) 3.82 (3H, s) 3.23-3.59 (1H, m) 0.95 (9H, s) 0.17 (6H, s)

Evaluation of CL1-186 Stability and In Vitro Metabolism

Rationale. Similar to N,N′-disubstituted aryl analogs shown above, N,N′-disubstituted branched aliphatic analogs of FTC, such as CL1-186 below, serve as model compounds to study activation kinetics of this functional group potential A2 monomers for strategy 3. A model compound of this type was generated (CL1-186).

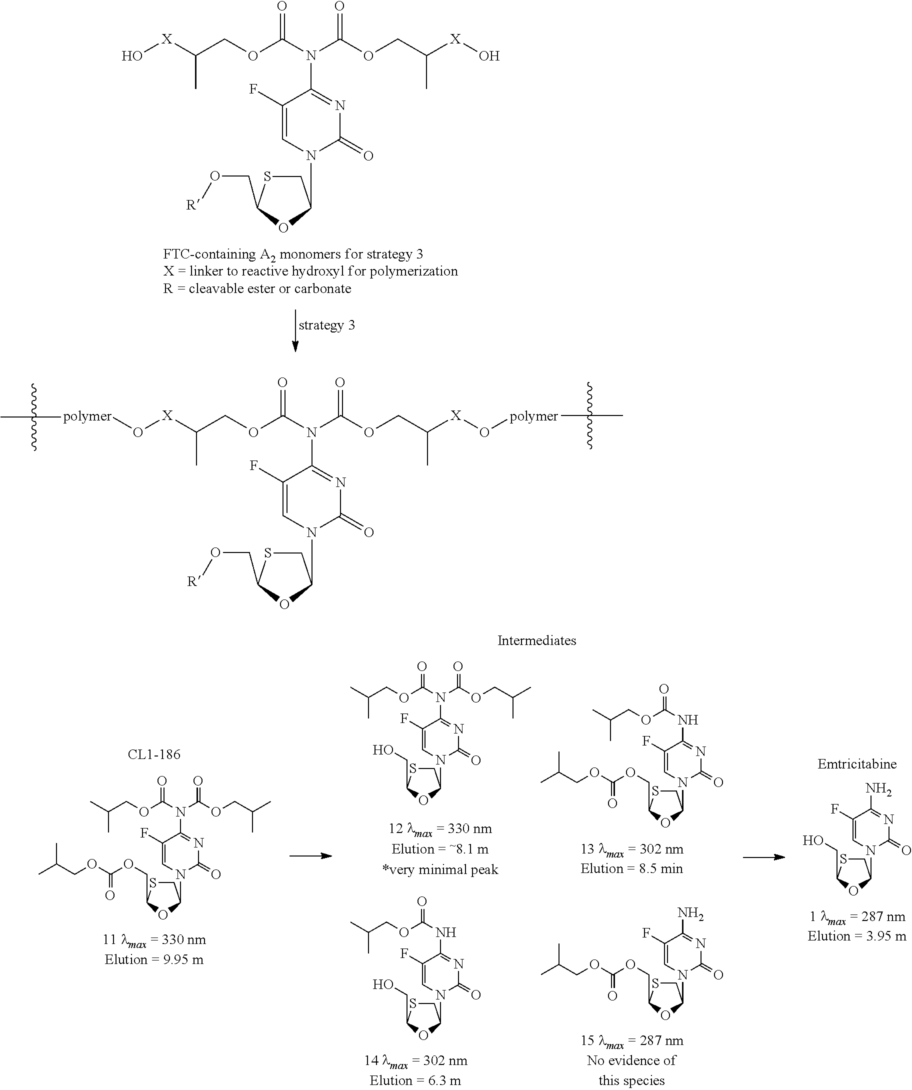

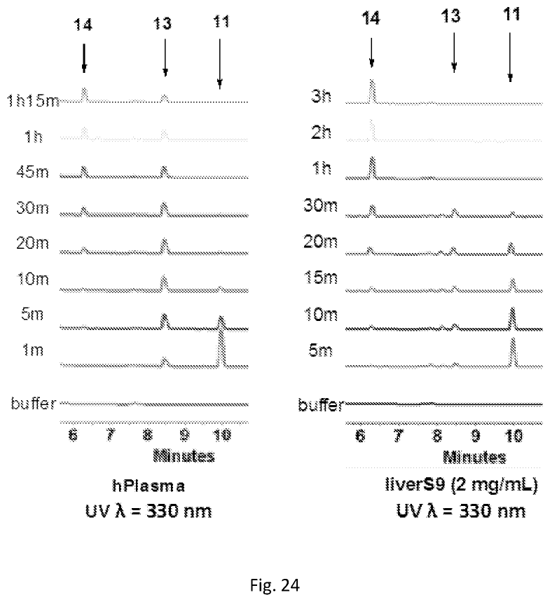

General Procedure for in vitro metabolism studies of CL1-186 via HPLC-UV: Experiments have been conducted to study the degradation profile of CL1-186 in liver S9 fraction and in human plasma. Reaction mixtures containing 95 μL phosphate buffer (0.1 M, pH 7.4), mixed pooled gender human liver S9 (2 mg/mL), or mixed gender human plasma are preincubated at 37° C. for 10 min. Reactions are initiated by the addition 5 μL of emtricitabine derivative (20 mM stock in DMSO). After incubation at 37° C., aliquots were taken at each time point and quenched in 2 volumes of ice-cold methanol. The quenched aliquots were then centrifuged at 14 000 rpm for 5 min. The supernatant was diluted 10-fold into phosphate buffer (0.1 M, pH 7.4) and injected onto the HPLC for analysis (FIG. 24) (5% to 100% B over 8 minutes at a flow rate of 1 mL min-1; solvent A: 50 mM triethylammonium acetate, pH 8; solvent B: acetonitrile) and read spectrophotometrically monitoring a decrease in initial peak area of CL1-186 at 330 nm or appearance and increase of the peak area of 1 at 287 nm. Tentative peak assignment of 13 is made on the basis of the change in chromophore and retention time relative to 11 (CL1-186) and FTC. The possible degradation pathway is shown below.

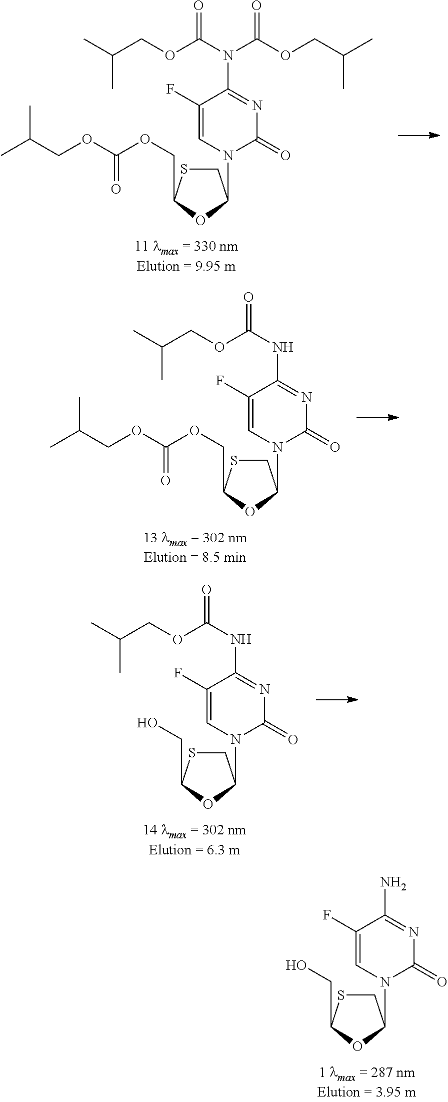

### BIBLIOGRAPHY AND REFERENCES CITED

1. UNAIDS. Fact sheet 2016. http://www.unaids.org/en/resources/fact-sheet. Updated 2016.
2. Palella F J, Jr, Baker R K, Moorman A C, et al. Mortality in the highly active antiretroviral therapy era: Changing causes of death and disease in the HIV outpatient study. JAIDS J Acquired Immune Defic Syndromes. 2006; 43(1).
3. Leyro T M, Vujanovic A A, Bonn-Miller M. Examining associations between cognitive-affective vulnerability and HIV symptom severity, perceived barriers to treatment adherence, and viral load among HIV-positive adults. Int J Behav Med. 2015; 22(1):139-148.
4. Panel on antiretroviral guidelines for adults and adolescents. guidelines for the use of antiretroviral agents in HIV-1-infected adults and adolescents. Department of Human and Health Services; 2015.
5. Granger B B, Bosworth H B. Medication adherence: Emerging use of technology. Curr Opin Cardiol. 2011; 26(4).
6. WHO Library Cataloguing-in-Publication Data. Adherence to long-term therapies: Evidence for action. 2003.
7. Spreen W, Ford S, Chen S, et al. Pharmacokinetics, safety and tolerability of the HIV integrase inhibitor S/GSK1265744 long acting parenteral nanosuspension following single dose administration to healthy adults. 2012.
8. Baert L, van't Klooster G, Dries W, et al. Development of a long-acting injectable formulation with nanoparticles of rilpivirine (TMC278) for HIV treatment. European Journal of Pharmaceutics and Biopharmaceutics. 2009; 72(3):502-508.
9. van't Klooster G, Hoeben E, Borghys H, et al. Pharmacokinetics and disposition of rilpivirine (TMC278) nanosuspension as a long-acting injectable antiretroviral formulation. Antimicrobial Agents and Chemotherapy. 2010; 54(5):2042-2050.
10. Margolis Dea. Cabotegravir+rilpivirine as long-acting maintenance therapy: LATTE-2 week 48 results. 2016.
11. Rohini N A, Joseph A, Mukerjil A. Polymeric prodrugs: Recent achievements and general strategies. Journal of Antivirals and Antiretrovirals. 2013(Special Issue):S15.
12. Duncan R. Polymer therapeutics: Top 10 selling pharmaceuticals—what next? J Controlled Release. 2014; 190:371-380.
13. Duncan R. Polymer conjugates as anticancer nanomedicines. Nat Rev Cancer. 2006; 6(9):688-701.
14. Pérez-Herrero E, Fernández-Medarde A. Advanced targeted therapies in cancer: Drug nanocarriers, the future of chemotherapy. European Journal of Pharmaceutics and Biopharmaceutics. 2015; 93:52-79.
15. Erdmann L, Macedo B, Uhrich K E. Degradable poly(anhydride ester) implants: Effects of localized salicylic acid release on bone. Biomaterials. 2000; 21(24):2507-2512.
16. Rosario-Melendez R, Harris C L, Delgado-Rivera R, Yu L, Uhrich K E. PolyMorphine: An innovative biodegradable polymer drug for extended pain relief. J Controlled Release. 2012; 162(3):538-544.
17. Danial M, Klok H A. Polymeric anti-HIV therapeutics. Macromol Biosci. 2015; 15(1):9-35.
18. Danial M, van Dulmen, Tim H. H., Aleksandrowicz J, Potgens A J G, Klok H. Site-specific PEGylation of HR2 peptides: Effects of PEG conjugation position and chain length on HIV-1 membrane fusion inhibition and proteolytic degradation. Bioconjugate Chem. 2012; 23(8):1648-1660.
19. Asano S, Gavrilyuk J, Burton D R, Barbas C F. Preparation and activities of macromolecule conjugates of the CCR5 antagonist maraviroc. ACS Med Chem Lett. 2014; 5(2):133-137.
20. Zuwala K, Smith A A, Postma A, et al. Polymers fight HIV: Potent (pro)drugs identified through parallel automated synthesis. Adv Healthc Mater. 2015; 4(1):46-50.
21. Margolis D, Bhatti L, Smith G, et al. Once-daily oral GSK1265744 (GSK744) as part of combination therapy in antiretroviral naïve adults: 24-week safety and efficacy results from the LATTE study (LAI1116482). 2013.
22. Cohen C J, Andrade-Villanueva J, Clotet B, et al. Rilpivirine versus efavirenz with two background nucleoside or nucleotide reverse transcriptase inhibitors in treatment-naive adults infected with HIV-1 (THRIVE): A phase 3, randomised, non-inferiority trial. The Lancet. 2011; 378(9787):229-237.
23. Benagiano G, Gabelnick H, Farris M. Contraceptive devices: Subcutaneous delivery systems. Expert Rev Med Devices. 2008; 5(5):623-637.
24. Chang M, Zhang F, Wei T, et al. Smart linkers in polymer-drug conjugates for tumor-targeted delivery. J Drug Target. 2016; 24(6):475-491.
25. Turecek P L, Bossard M J, Schoetens F, Ivens I A. PEGylation of biopharmaceuticals: A review of chemistry and nonclinical safety information of approved drugs. J Pharm Sci. 2016; 105(2):460-475.
26. Carmichael J B. Step-reaction polymerization. In: Mark H F, ed. Encyclopedia of polymer science and technology. 4th ed.; 2014:82.
27. Elizade L, De Los Santos-Villarreal G, Santiago-Garcia J, Aguilar-Vega M. Step-growth polymerization. In: Saldivar-Guerra E, Vivaldo-Lima E, eds. Handbook of polymer synthesis, characterization, and processing.; 2013:43.
28. Dove A P, Meier M A R. Step-growth polymerization in the 21st century. Macromolecular Chemistry and Physics. 2014; 215(22):2135-2137.
29. Shukla S K, Shukla S K, Govender P P, Giri N G. Biodegradable polymeric nanostructures in therapeutic applications: Opportunities and challenges. RSC Adv. 2016; 6(97):94325-94351.
30. Singh J, Desai S, Yadav S, Narasimhan B, Kaur H. Polymer drug conjugates: Recent advancements in various diseases. Curr Pharm Des. 2016; 22(19):2821-2843.
31. Pola R, Heinrich A K, Mueller T, et al. Passive tumor targeting of polymer therapeutics: In vivo imaging of both the polymer carrier and the enzymatically cleavable drug model. Macromol Biosci. 2016; 16(11):1577-1582.
32. Ray A S, Fordyce M W, Hitchcock M J M. Tenofovir alafenamide: A novel prodrug of tenofovir for the treatment of human immunodeficiency virus. Antiviral Res. 2016; 125:63-70.
33. Stoddart C A, Galkina S A, Joshi P, et al. Oral administration of the nucleoside EFdA (4′-ethynyl-2-fluoro-2′-deoxyadenosine) provides rapid suppression of HIV viremia in humanized mice and favorable pharmacokinetic properties in mice and the rhesus macaque. Antimicrob Agents Chemother. 2015; 59(7):4190-4198.
34. Levine M N, Raines R T. Trimethyl lock: A trigger for molecular release in chemistry, biology, and pharmacology. Chem Sci. 2012; 3(8):2412-2420.
35. Greenwald R B, Choe Y H, Conover C D, Shum K, Wu D, Royzen M. Drug delivery systems based on trimethyl lock lactonization: poly(ethylene glycol) prodrugs of amino-containing compounds. J Med Chem. 2000; 43(3):475-487.
36. Wang L H, Begley J, St Claire R L 3rd, Harris J, Wakeford C, Rousseau F S. Pharmacokinetic and pharmacodynamic characteristics of emtricitabine support its once daily dosing for the treatment of HIV infection. AIDS Research and Human Retroviruses. 2004; 20(11):1173-1182.
37. Zhang H, Zhou Y, Alcock C, et al. Novel single-cell-level phenotypic assay for residual drug susceptibility and reduced replication capacity of drug-resistant human immunodeficiency virus type 1. J Virol. 2004; 78(4):1718-1729.
38. Shen L, Peterson S, Sedaghat A R, et al. Dose-response curve slope sets class-specific limits on inhibitory potential of anti-HIV drugs. Nat Med. 2008; 14(7):762-766.
39. Aukland K, Reed R K. Interstitial-lymphatic mechanisms in the control of extracellular fluid volume. Physiol Rev. 1993; 73(1):1-78.
40. Fogh-Andersen N, Altura B M, Altura B T, Siggaard-Andersen O. Composition of interstitial fluid. Clin Chem. 1995; 41(10):1522-1525.
41. Giardiello M, Hatton F L, Slater R A, et al. Stable, polymer-directed and SPION-nucleated magnetic amphiphilic block copolymer nanoprecipitates with readily reversible assembly in magnetic fields. Nanoscale. 2016; 8(13):7224-7231.
42. Hatton F L, Chambon P, Savage A C, Rannard S P. Role of highly branched, high molecular weight polymer structures in directing uniform polymer particle formation during nanoprecipitation. Chem Commun (Camb). 2016; 52(20):3915-3918.
43. Rogers H E, Chambon P, Auty S E R, Hem F Y, Owen A, Rannard S P. Synthesis, nanoprecipitation and pH sensitivity of amphiphilic linear-dendritic hybrid polymers and hyperbranched-polydendrons containing tertiary amine functional dendrons. Soft Matter. 2015; 11(35):7005-7015
44. Ford J, Chambon P, North J, et al. Multiple and co-nanoprecipitation studies of branched hydrophobic copolymers and Aâ€″B amphiphilic block copolymers, allowing rapid formation of sterically stabilized nanoparticles in aqueous media. Macromolecules. 2015; 48(6):1883-1893.
45. Hatton F L, Tatham L M, Tidbury L R, et al. Hyperbranched polydendrons: A new nanomaterials platform with tuneable permeation through model gut epithelium. Chem Sci. 2015; 6(1):326-334.
46. Hatton F L, Chambon P, McDonald T O, Owen A, Rannard S P. Hyperbranched polydendrons: A new controlled macromolecular architecture with self-assembly in water and organic solvents. Chem Sci. 2014; 5(5):1844-1853.
47. Ranganath S H, Kee I, Krantz W B, Chow P K, Wang C. Hydrogel matrix entrapping PLGA-paclitaxel microspheres: Drug delivery with near zero-order release and implantability advantages for malignant brain tumour chemotherapy. Pharm Res. 2009; 26(9):2101-2114.
48. Crawford R J, Paul D. Solid phase compaction of polymers. J Mater Sci. 1979; 14(11):2693-2702.
49. Crawford R J, Paul D W. Cold compaction of polymeric powders. J Mater Sci. 1982; 17(8):2267-2280.
50. Potter D S, Potter C D O, inventors. Projectile drug delivery technology. US patent, US 20030054044, 2003. Contact PD/PI: Freel Meyers, Caren L References Cited Page 115
51. Potter C D O, Nabahi S, inventors. Solid pharmaceutical and vaccine dose. World patent, WO 2008102136, 2008.
52. Potter C, Watson J, inventors. Drug delivery technology comprising solid dose injection devices. World patent, WO 2012098356, 2012.
53. Abrahams R A, Ronel S H. Biocompatible implants for the sustained zero-order release of narcotic antagonists. J Biomed Mater Res. 1975; 9(3):355-366.
54. Kong Q, Wei M, Su H, Yu J, Jiang J, inventors. Carmustine sustained-release implant for treatment of solid tumors and preparation method thereof. CN patent, CN 104523566 A, Apr. 22, 2015.
55. Rajoli R R, Back D, Rannard S, et al. Physiologically based pharmacokinetic modelling to inform development of intramuscular long-acting nanoformulations for HIV. Clin Pharmacokinet. 2015; 54(6):639-650.
56. Jamel M, Turner D, Yang J, et al. Population-based mechanistic prediction of oral drug absorption. The AAPS Journal. 2009; 11(2):225-237.
57. Martin P, Giardiello M, McDonald T O, et al. Augmented inhibition of CYP3A4 in human primary hepatocytes by ritonavir solid drug nanoparticles. Mol Pharm. 2015; 12(10):3556-3568.
58. Moss D M, Liptrott N J, Siccardi M, Owen A. Interactions of antiretroviral drugs with the SLC22A1 (OCT1) drug transporter. Front Pharmacol. 2015; 6:78.
59. Moss D M, Curley P, Shone A, Siccardi M, Owen A. A multisystem investigation of raltegravir association with intestinal tissue: Implications for pre-exposure prophylaxis and eradication. J Antimicrob Chemother. 2014; 69(12):3275-3281.
60. Curley P, Rajoli R K, Moss D M, et al. Efavirenz is predicted to accumulate in brain tissue: An in silico, in vitro and in vivo investigation. Antimicrob Agents Chemother. 2016.
61. Giardiello M, Liptrott N J, McDonald T O, et al. Accelerated oral nanomedicine discovery from miniaturized screening to clinical production exemplified by paediatric HIV nanotherapies. Nat Commun. 2016; 7:13184.
62. McDonald T O, Giardiello M, Martin P, et al. Antiretroviral solid drug nanoparticles with enhanced oral bioavailability: Production, characterization, and in vitro-in vivo correlation. Advanced Healthcare Materials. 2014; 3(3):400-411.
63. Liptrott N J, Curley P, Moss D, Back D J, Khoo S H, Owen A. Interactions between tenofovir and nevirapine in CD4+ T cells and monocyte-derived macrophages restrict their intracellular accumulation. J Antimicrob Chemother. 2013; 68(11):2545-2549.
64. McDonald T O, Martin P, Patterson J P, et al. Multicomponent organic nanoparticles for fluorescence studies in biological systems. Advanced Functional Materials. 2012; 22(12):2469-2478.
65. Bahar F G, Ohura K, Ogihara T, Imai T. Species difference of esterase expression and hydrolase activity in plasma. J Pharm Sci. 2012; 101(10):3979-3988.
66. Liptrott N J, Giardiello M, Hunter J W, et al. Flow cytometric analysis of the physical and protein-binding characteristics of solid drug nanoparticle suspensions. Nanomedicine (Lond). 2015; 10(9):1407-1421.
67. David C A, Owen A, Liptrott N J. Determining the relationship between nanoparticle characteristics and immunotoxicity: Key challenges and approaches. Nanomedicine (Lond). 2016; 11(11):1447-1464. Contact PD/PI: Freel Meyers, Caren L References Cited Page 116
68. Yu H, Wang Z, Sun G, Yu Y. Recognition of nucleic acid ligands by toll-like receptors 7/8: Importance of chemical modification. Curr Med Chem. 2012; 19(9):1365-1377.
69. Jurk M, Kritzler A, Schulte B, et al. Modulating responsiveness of human TLR7 and 8 to small molecule ligands with T-rich phosphorothiate oligodeoxynucleotides. Eur J Immunol. 2006; 36(7):1815-1826.
70. Padmore T, Stark C, Turkevich L A, Champion J A. Quantitative analysis of the role of fiber length on phagocytosis and inflammatory response by alveolar macrophages. Biochimica et Biophysica Acta (BBA)—General Subjects. 2017; 1861(2):58-67.
71. Marzolini C, Rajoli R, Battegay M, Elzi L, Back D, Siccardi M. Physiologically based pharmacokinetic modeling to predict drug-drug interactions with efavirenz involving simultaneous inducing and inhibitory effects on cytochromes. Clin Pharmacokinet. 2016.
72. Siccardi M, Olagunju A, Seden K, et al. Use of a physiologically-based pharmacokinetic model to simulate artemether dose adjustment for overcoming the drug-drug interaction with efavirenz. In Silico Pharmacol. 2013; 1:10.1186/2193-9616-1-4.
73. Molto J, Rajoli R, Back D, et al. Use of a physiologically-based pharmacokinetic model to simulate drugdrug interactions between antineoplastic and antiretroviral drugs. Journal of Antimicrobial Chemotherapy. In Press.
74. Gunawardana M, Remedios-Chan M, Miller C S, et al. Pharmacokinetics of long-acting tenofovir alafenamide (GS-7340) subdermal implant for HIV prophylaxis. Antimicrob Agents Chemother. 2015; 59(7):3913-3919.
75. Rajca A, Li Q, Date A, Belshan M, Destache C. Thermosensitive vaginal gel containing PLGA-NRTI conjugated nanoparticles for HIV prophylaxis. 2013; 3:293.
76. Sharma A, Jain S, Modi M, Vashisht V, Singh H. Recent advances in NDDS (novel drug delivery systems) for delivery of anti-HIV drugs. Research Journal of Pharmaceutical, Biological and Chemical Sciences. 2010; 1(3):78-88.
101. J. V Olsson, D. Hult, S. Garcia-Gallego and M. Malkoch, Chem. Sci., 2017, 8, 4853-4857.
102. S. P. Rannard and N. J. Davis, Org. Lett., 1999, 1, 933-936.
103. H. Ihre, A. Hult, J. M. J. Fréchet and I. Gitsov, Macromolecules, 1998, 31, 4061-4068.
104. M. Malkoch, E. Malmström and A. Hult, Macromolecules, 2002, 35, 8307-8314.
201. Jackson A, Moyle G, Watson V, Tjia J, Ammara A, Back D, et al. Tenofovir, emtricitabine intracellular and plasma, and efavirenz plasma concentration decay following drug intake cessation: implications for HIV treatment and prevention. JAIDS Journal of Acquired Immune Deficiency Syndromes. 2013; 62(3):275-81.
202. Gamboa A, O'Barr S. Using the wax moth larvae as a model to study the immune system. Garey High School Pomona Calif., Lab protocol. 2016.
301. Drug Delivery Systems Based on Trimethyl Lock Lactonization: Poly(ethylene glycol) Prodrugs of Amino-Containing Compounds, Richard B. Greenwald, Yun H. Choe, Charles D. Conover, Kwok Shum, Dechun Wu, and Maksim Royzen, J. Med. Chem. 2000, 43, 475-487.
302. NMI/MsCl-Mediated Amide Bond Formation of Aminopyrazines and Aryl/Heteroaryl Carboxylic Acids: Synthesis of Biologically Relevant Pyrazine Carboxamides, Nagaraja Reddy Gangarapu, Eeda Koti Reddy, Ayyiliath M Sajith, Shivaraj Yellappa and Kothapalli Bannoth Chandrasekhar, ChemistrySelect 2017, 2, 7706-7710.
303. The Uronium/Guanidinium Peptide Coupling Reagents: Finally the True Uronium Salts, Louis A. Carpino, Hideko Imazumi, Ayman El-Faham, Fernando J. Ferrer, Chongwu Zhang, Yunsub Lee, Bruce M. Foxman, Peter Henklein, Christiane Hanay, Clemens M,gge, Holger Wenschuh, Jana Klose, Michael Beyermann, and Michael Bienert, Angew. Chem. Int. Ed. 2002, 41 (3), 441-444.
304. Mechanism for Effective Lymphoid Cell and Tissue Loading Following Oral Administration of Nucleotide Prodrug GS-7340, Darius Babusis, Truc K. Phan, William A. Lee, William J. Watkins, and Adrian S. Ray, Mol. Pharmaceutics 2013, 10, 459-466.
305. Metabolism of GS-7340, a novel phenyl Monophosphoramidate intracellular prodrug of PMPA, in blood, Nucleosides, Nucleotides & Nucleic Acids, 2001, 20(4-7), 1091-1098.
306. Pirrung, M. C., Shuey, S. W., Lever, D. C., & Fallon, L. (1994). A convenient procedure for the deprotection of silylated nucleosides and nucleotides using triethylamine trihydrofluoride. Bioorganic & Medicinal Chemistry Letters, 4(11), 1345-1346.
307. Li, F., Jas, G. S., Qin, G., Li, K., & Li, Z. (2010). Synthesis and evaluation of bivalent, peptidomimetic antagonists of the αvβ3 integrins. Bioorganic & medicinal chemistry letters, 20(22), 6577-6580.

1. A product which is a prodrug of a nucleoside reverse transcriptase inhibitor (NRTI) in the form of a polymer.

2. A product as claimed in claim 1 which is a polymeric NRTI delivery system comprising a polymeric material which is capable of degradation after administration to release an NRTI or NRTI prodrug which itself is capable of metabolism to the parent NRTI.

3. A product as claimed in claim 1 wherein the NRTI is selected from FTC, 3TC, EFdA and TFV.

4. A product as claimed in claim 1 wherein the NRTI is incorporated into the polymer via an amino group of the NRTI.

5. A product as claimed in claim 4 wherein said incorporation forms carbamate or amide linkages.

6. A product as claimed in claim 1 wherein the NRTI is incorporated into the polymer via a hydroxy or oxy group of the NRTI.

7. A product as claimed in claim 6 wherein said incorporation forms carbonate or ester linkages.

8. A product as claimed in claim 1 wherein the NRTI is incorporated into the polymer via a phosphonyl group of the NRTI.

9. A product as claimed in claim 1 wherein the polymeric structure comprises linkers between carbonates, esters, carbamates and/or amides.

10. A product as claimed in claim 9 wherein said linkers are or comprise alkyl chains, or aromatic or heteroaromatic linkers

11. A product as claimed in claim 1 wherein the NRTI is present on the polymer as a pendant moiety.

12. A method of preparing a product of claim 1 comprising incorporating an NRTI or derivative thereof into a polymer.

13. A method as claimed in claim 12 comprising reaction with a monomer which is able to react with amines or alcohols to form carbamates, carbonates, amides or esters.

14. A method as claimed in claim 13 wherein said monomer is bifunctional.

15. A method as claimed in claim 14 wherein said monomer is a bis(chloroformate).

16. A method as claimed in claim 12 comprising reaction with a multivalent compound which is able to act as a brancher.

17. A method as claimed in claim 16 wherein said multivalent compound is a polyol.

18. A method as claimed in claim 12 comprising linking of monomers using imidazole or triazole chemistry, for example using CDI.

19. A construct of a product of claim 1 in the form of an injectable composition.

20. A construct of a product of claim 1 in the form of an implant.

21. A method of treatment comprising administration of a product as claimed in claim 1 to a patient in need thereof.

22. (canceled)

23. A method of treatment of HIV comprising administration of a product as claimed in claim 1 to a patient in need thereof.

24. A product which is a polymeric NRTI delivery system comprising a polymeric material which is capable of degradation after administration to release an NRTI or NRTI prodrug which itself is capable of metabolism to the parent NRTI.
